SEQUENCE LISTING (620 sequences)

SEQ ID NO: 1 - DNA, 30 bp, Artificial Sequence
  Description of Artificial Sequence: Synthetic oligonucleotide
aaaaaaaaaaaaaacgttaaaaaaaaaaaa

SEQ ID NO: 2 - DNA, 30 bp, Artificial Sequence; identical to SEQ ID NO: 1
  Description of Artificial Sequence: Synthetic DNA/RNA oligonucleotide

SEQ ID NO: 3 - DNA, 30 bp, Artificial Sequence
  Description of Artificial Sequence: Synthetic DNA/RNA oligonucleotide
aaaaaaaaaaaaaacguuaaaaaaaaaaaa

SEQ ID NO: 4 - RNA, 30 nt, Artificial Sequence
  Description of Artificial Sequence: Synthetic RNA oligonucleotide
aaaaaaaaaaaaaacguuaaaaaaaaaaaa

SEQ ID NO: 5 - DNA, 30 bp, Artificial Sequence
  Description of Artificial Sequence: Synthetic DNA/RNA oligonucleotide
ggggggggggggaacgttgggggggggggg

SEQ ID NO: 6 - DNA, 30 bp, Artificial Sequence
  Description of Artificial Sequence: Synthetic DNA/RNA oligonucleotide
aaaaaaaaaaaacccgggaaaaaaaaaaaa

SEQ ID NO: 7 - DNA, 30 bp, Artificial Sequence
  Description of Artificial Sequence: Synthetic DNA/RNA oligonucleotide
aaaaaaaaaaaaaacgaaaaaaaaaaaaaa

SEQ ID NO: 8 - DNA, 30 bp, Artificial Sequence
  Description of Artificial Sequence: Synthetic DNA/RNA oligonucleotide
ggggggggggggaacguugggggggggggg

SEQ ID NO: 9 - skipped

SEQ ID NO: 10 - RNA, 30 nt, Artificial Sequence
  Description of Artificial Sequence: Synthetic RNA oligonucleotide
ggggggggggggaacguugggggggggggg

SEQ ID NO: 11 - DNA, 30 bp, Artificial Sequence
  Description of Artificial Sequence: Synthetic oligonucleotide
aaaaaaaaaaaaaagcttaaaaaaaaaaaa

SEQ ID NO: 12 - DNA, 14 bp, Artificial Sequence
  Description of Artificial Sequence: Synthetic HDR
tcaacgttaacgtt

SEQ ID NO: 13 - DNA, 26 bp, Artificial Sequence
  Description of Artificial Sequence: Synthetic HDR
tccatgacgttcctgatgctaacgtt

SEQ ID NO: 14 - DNA, 26 bp, Artificial Sequence
  Description of Artificial Sequence: Synthetic HDR
atcgactctcgagcgttctcaacgtt

SEQ ID NO: 15 - DNA, 21 bp, Artificial Sequence
  Description of Artificial Sequence: Synthetic HDR
gcatgacgttgagctaacgtt

SEQ ID NO: 16 - DNA, 14 bp, Artificial Sequence
  Description of Artificial Sequence: Synthetic HDR
tcagcgctaacgtt

SEQ ID NO: 17 - DNA, 26 bp, Artificial Sequence
  Description of Artificial Sequence: Synthetic HDR
gagaacgctggaccttccataacgtt

SEQ ID NO: 18 - DNA, 26 bp, Artificial Sequence
  Description of Artificial Sequence: Synthetic HDR
gagaacgctcgaccttccataacgtt

SEQ ID NO: 19 - DNA, 26 bp, Artificial Sequence
  Description of Artificial Sequence: Synthetic HDR
gagaacgctcgaccttcgataacgtt

SEQ ID NO: 20 - DNA, 26 bp, Artificial Sequence
  Description of Artificial Sequence: Synthetic HDR
gagaacgctccagcactgataacgtt

SEQ ID NO: 21 - DNA, 26 bp, Artificial Sequence
  Description of Artificial Sequence: Synthetic HDR
tccatgtcggtcctgatgctaacgtt

SEQ ID NO: 22 - DNA, 26 bp, Artificial Sequence
  Description of Artificial Sequence: Synthetic HDR
tccatgtcggtcctgctgataacgtt

SEQ ID NO: 23 - DNA, 26 bp, Artificial Sequence
  Description of Artificial Sequence: Synthetic HDR
atggactctccagcgttctcaacgtt

SEQ ID NO: 24 - DNA, 26 bp, Artificial Sequence
  Description of Artificial Sequence: Synthetic HDR
atggaaggtccaacgttctcaacgtt

SEQ ID NO: 25 - DNA, 15 bp, Artificial Sequence
  Description of Artificial Sequence: Synthetic HDR
gctagacgttagcgt

SEQ ID NO: 26 - DNA, 8 bp, Artificial Sequence
  Description of Artificial Sequence: Synthetic HDR
tcaacgtt

SEQ ID NO: 27 - DNA, 20 bp, Artificial Sequence
  Description of Artificial Sequence: Synthetic HDR
tccatgacgttcctgatgct

SEQ ID NO: 28 - DNA, 20 bp, Artificial Sequence
  Description of Artificial Sequence: Synthetic HDR
atcgactctcgagcgttctc

SEQ ID NO: 29 - DNA, 15 bp, Artificial Sequence
  Description of Artificial Sequence: Synthetic HDR
gcatgacgttgagct

SEQ ID NO: 30 - DNA, 8 bp, Artificial Sequence
  Description of Artificial Sequence: Synthetic HDR
tcagcgct

SEQ ID NO: 31 - DNA, 20 bp, Artificial Sequence
  Description of Artificial Sequence: Synthetic HDR
gagaacgctggaccttccat

SEQ ID NO: 32 - DNA, 20 bp, Artificial Sequence
  Description of Artificial Sequence: Synthetic HDR
gagaacgctcgaccttccat

SEQ ID NO: 33 - DNA, 20 bp, Artificial Sequence
  Description of Artificial Sequence: Synthetic HDR
gagaacgctcgaccttcgat

SEQ ID NO: 34 - DNA, 20 bp, Artificial Sequence
  Description of Artificial Sequence: Synthetic HDR
gagaacgctccagcactgat

SEQ ID NO: 35 - DNA, 20 bp, Artificial Sequence
  Description of Artificial Sequence: Synthetic HDR
tccatgtcggtcctgatgct

SEQ ID NO: 36 - DNA, 20 bp, Artificial Sequence
  Description of Artificial Sequence: Synthetic HDR
tccatgtcggtcctgctgat

SEQ ID NO: 37 - DNA, 20 bp, Artificial Sequence
  Description of Artificial Sequence: Synthetic HDR
atggactctccagcgttctc

SEQ ID NO: 38 - DNA, 20 bp, Artificial Sequence
  Description of Artificial Sequence: Synthetic HDR
atggaaggtccaacgttctc

SEQ ID NO: 39 - DNA, 20 bp, Artificial Sequence
  Description of Artificial Sequence: Synthetic HDR
tccatggcggtcctgatgct

SEQ ID NO: 40 - DNA, 20 bp, Artificial Sequence
  Description of Artificial Sequence: Synthetic HDR
tccatgacggtcctgatgct

SEQ ID NO: 41 - DNA, 20 bp, Artificial Sequence
  Description of Artificial Sequence: Synthetic HDR
tccatgtcgatcctgatgct

SEQ ID NO: 42 - DNA, 20 bp, Artificial Sequence
  Description of Artificial Sequence: Synthetic HDR
tccatgtcgctcctgatgct

SEQ ID NO: 43 - DNA, 20 bp, Artificial Sequence
  Description of Artificial Sequence: Synthetic HDR
tccatgtcgttcctgatgct

SEQ ID NO: 44 - DNA, 20 bp, Artificial Sequence
  Description of Artificial Sequence: Synthetic HDR
tccataacgttcctgatgct

SEQ ID NO: 45 - DNA, 20 bp, Artificial Sequence
  Description of Artificial Sequence: Synthetic HDR
tccatgacgtccctgatgct

SEQ ID NO: 46 - DNA, 26 bp, Artificial Sequence
  Description of Artificial Sequence: Synthetic HDR
tccatggcggtcctgatgctaacgtt

SEQ ID NO: 47 - DNA, 26 bp, Artificial Sequence
  Description of Artificial Sequence: Synthetic HDR
tccatgacggtcctgatgctaacgtt

SEQ ID NO: 48 - DNA, 26 bp, Artificial Sequence
  Description of Artificial Sequence: Synthetic HDR
tccatgtcgatcctgatgctaacgtt

SEQ ID NO: 49 - DNA, 26 bp, Artificial Sequence
  Description of Artificial Sequence: Synthetic HDR
tccatgtcgctcctgatgctaacgtt

SEQ ID NO: 50 - DNA, 26 bp, Artificial Sequence
  Description of Artificial Sequence: Synthetic HDR
tccatgtcgttcctgatgctaacgtt

SEQ ID NO: 51 - DNA, 26 bp, Artificial Sequence
  Description of Artificial Sequence: Synthetic HDR
tccataacgttcctgatgctaacgtt

SEQ ID NO: 52 - DNA, 26 bp, Artificial Sequence
  Description of Artificial Sequence: Synthetic HDR
tccatgacgtccctgatgctaacgtt

SEQ ID NO: 53 - DNA, 23 bp, Artificial Sequence
  Description of Artificial Sequence: Synthetic HDR
gctagacgttagcgtacaacgtt

SEQ ID NO: 54 - DNA, 16 bp, Artificial Sequence
  Description of Artificial Sequence: Synthetic HDR
tcaacgttacaacgtt

SEQ ID NO: 55 - DNA, 28 bp, Artificial Sequence
  Description of Artificial Sequence: Synthetic HDR
tccatgacgttcctgatgctacaacgtt

SEQ ID NO: 56 - DNA, 28 bp, Artificial Sequence
  Description of Artificial Sequence: Synthetic HDR
atcgactctcgagcgttctcacaacgtt

SEQ ID NO: 57 - DNA, 23 bp, Artificial Sequence
  Description of Artificial Sequence: Synthetic HDR
gcatgacgttgagctacaacgtt

SEQ ID NO: 58 - DNA, 16 bp, Artificial Sequence
  Description of Artificial Sequence: Synthetic HDR
tcagcgctacaacgtt

SEQ ID NO: 59 - DNA, 28 bp, Artificial Sequence
  Description of Artificial Sequence: Synthetic HDR
gagaacgctggaccttccatacaacgtt

SEQ ID NO: 60 - DNA, 28 bp, Artificial Sequence
  Description of Artificial Sequence: Synthetic HDR
gagaacgctcgaccttccatacaacgtt

SEQ ID NO: 61 - DNA, 28 bp, Artificial Sequence
  Description of Artificial Sequence: Synthetic HDR
gagaacgctcgaccttcgatacaacgtt

SEQ ID NO: 62 - DNA, 28 bp, Artificial Sequence
  Description of Artificial Sequence: Synthetic HDR
gagaacgctccagcactgatacaacgtt

SEQ ID NO: 63 - DNA, 28 bp, Artificial Sequence
  Description of Artificial Sequence: Synthetic HDR
tccatgtcggtcctgatgctacaacgtt

SEQ ID NO: 64 - DNA, 28 bp, Artificial Sequence
  Description of Artificial Sequence: Synthetic HDR
tccatgtcggtcctgctgatacaacgtt

SEQ ID NO: 65 - DNA, 28 bp, Artificial Sequence
  Description of Artificial Sequence: Synthetic HDR
atggactctccagcgttctcacaacgtt

SEQ ID NO: 66 - DNA, 28 bp, Artificial Sequence
  Description of Artificial Sequence: Synthetic HDR
atggaaggtccaacgttctcacaacgtt

SEQ ID NO: 67 - DNA, 28 bp, Artificial Sequence
  Description of Artificial Sequence: Synthetic HDR
tccatggcggtcctgatgctacaacgtt

SEQ ID NO: 68 - DNA, 28 bp, Artificial Sequence
  Description of Artificial Sequence: Synthetic HDR
tccatgacggtcctgatgctacaacgtt

SEQ ID NO: 69 - DNA, 28 bp, Artificial Sequence
  Description of Artificial Sequence: Synthetic HDR
tccatgtcgatcctgatgctacaacgtt

SEQ ID NO: 70 - DNA, 28 bp, Artificial Sequence
  Description of Artificial Sequence: Synthetic HDR
tccatgtcgctcctgatgctacaacgtt

SEQ ID NO: 71 - DNA, 28 bp, Artificial Sequence
  Description of Artificial Sequence: Synthetic HDR
tccatgtcgttcctgatgctacaacgtt

SEQ ID NO: 72 - DNA, 23 bp, Artificial Sequence
  Description of Artificial Sequence: Synthetic HDR
acaacgttgctagacgttagcgt

SEQ ID NO: 73 - DNA, 16 bp, Artificial Sequence
  Description of Artificial Sequence: Synthetic HDR
acaacgtttcaacgtt

SEQ ID NO: 74 - DNA, 28 bp, Artificial Sequence
  Description of Artificial Sequence: Synthetic HDR
acaacgtttccatgacgttcctgatgct

SEQ ID NO: 75 - DNA, 28 bp, Artificial Sequence
  Description of Artificial Sequence: Synthetic HDR
acaacgttatcgactctcgagcgttctc

SEQ ID NO: 76 - DNA, 23 bp, Artificial Sequence
  Description of Artificial Sequence: Synthetic HDR
acaacgttgcatgacgttgagct

SEQ ID NO: 77 - DNA, 16 bp, Artificial Sequence
  Description of Artificial Sequence: Synthetic HDR
acaacgtttcagcgct

SEQ ID NO: 78 - DNA, 28 bp, Artificial Sequence
  Description of Artificial Sequence: Synthetic HDR
acaacgttgagaacgctggaccttccat

SEQ ID NO: 79 - DNA, 28 bp, Artificial Sequence
  Description of Artificial Sequence: Synthetic HDR
acaacgttgagaacgctcgaccttccat

SEQ ID NO: 80 - DNA, 28 bp, Artificial Sequence
  Description of Artificial Sequence: Synthetic HDR
acaacgttgagaacgctcgaccttcgat

SEQ ID NO: 81 - DNA, 28 bp, Artificial Sequence
  Description of Artificial Sequence: Synthetic HDR
acaacgttgagaacgctccagcactgat

SEQ ID NO: 82 - DNA, 28 bp, Artificial Sequence
  Description of Artificial Sequence: Synthetic HDR
acaacgtttccatgtcggtcctgatgct

SEQ ID NO: 83 - DNA, 28 bp, Artificial Sequence
  Description of Artificial Sequence: Synthetic HDR
acaacgtttccatgtcggtcctgctgat

SEQ ID NO: 84 - DNA, 28 bp, Artificial Sequence
  Description of Artificial Sequence: Synthetic HDR
acaacgttatggactctccagcgttctc

SEQ ID NO: 85 - DNA, 28 bp, Artificial Sequence
  Description of Artificial Sequence: Synthetic HDR
acaacgttatggaaggtccaacgttctc

SEQ ID NO: 86 - DNA, 28 bp, Artificial Sequence
  Description of Artificial Sequence: Synthetic HDR
acaacgtttccatggcggtcctgatgct

SEQ ID NO: 87 - DNA, 28 bp, Artificial Sequence
  Description of Artificial Sequence: Synthetic HDR
acaacgtttccatgacggtcctgatgct

SEQ ID NO: 88 - DNA, 28 bp, Artificial Sequence
  Description of Artificial Sequence: Synthetic HDR
acaacgtttccatgtcgatcctgatgct

SEQ ID NO: 89 - DNA, 28 bp, Artificial Sequence
  Description of Artificial Sequence: Synthetic HDR
acaacgtttccatgtcgctcctgatgct

SEQ ID NO: 90 - DNA, 28 bp, Artificial Sequence
  Description of Artificial Sequence: Synthetic HDR
acaacgtttccatgtcgttcctgatgct

SEQ ID NO: 91 - DNA, 15 bp, Artificial Sequence
  Description of Artificial Sequence: Synthetic HDR
gctagacgttagcgt

SEQ ID NO: 92 - DNA, 8 bp, Artificial Sequence
  Description of Artificial Sequence: Synthetic HDR
tcaacgtt

SEQ ID NO: 93 - DNA, 20 bp, Artificial Sequence
  Description of Artificial Sequence: Synthetic HDR
tccatgacgttcctgatgct

SEQ ID NO: 94 - DNA, 20 bp, Artificial Sequence
  Description of Artificial Sequence: Synthetic HDR
atcgactctcgagcgttctc

SEQ ID NO: 95 - DNA, 15 bp, Artificial Sequence
  Description of Artificial Sequence: Synthetic HDR
gcatgacgttgagct

SEQ ID NO: 96 - DNA, 8 bp, Artificial Sequence
  Description of Artificial Sequence: Synthetic HDR
tcagcgct

SEQ ID NO: 97 - DNA, 20 bp, Artificial Sequence
  Description of Artificial Sequence: Synthetic HDR
gagaacgctggaccttccat

SEQ ID NO: 98 - DNA, 20 bp, Artificial Sequence
  Description of Artificial Sequence: Synthetic HDR
gagaacgctcgaccttccat

SEQ ID NO: 99 - DNA, 20 bp, Artificial Sequence
  Description of Artificial Sequence: Synthetic HDR
gagaacgctcgaccttcgat

SEQ ID NO: 100 - DNA, 20 bp, Artificial Sequence
  Description of Artificial Sequence: Synthetic HDR
gagaacgctccagcactgat

SEQ ID NO: 101 - DNA, 20 bp, Artificial Sequence
  Description of Artificial Sequence: Synthetic HDR
tccatgtcggtcctgatgct

SEQ ID NO: 102 - DNA, 20 bp, Artificial Sequence
  Description of Artificial Sequence: Synthetic HDR
tccatgtcggtcctgctgat

SEQ ID NO: 103 - DNA, 20 bp, Artificial Sequence
  Description of Artificial Sequence: Synthetic HDR
atggactctccagcgttctc

SEQ ID NO: 104 - DNA, 20 bp, Artificial Sequence
  Description of Artificial Sequence: Synthetic HDR
atggaaggtccaacgttctc

SEQ ID NO: 105 - DNA, 20 bp, Artificial Sequence
  Description of Artificial Sequence: Synthetic HDR
tccatggcggtcctgatgct

SEQ ID NO: 106 - DNA, 20 bp, Artificial Sequence
  Description of Artificial Sequence: Synthetic HDR
tccatgacggtcctgatgct

SEQ ID NO: 107 - DNA, 20 bp, Artificial Sequence
  Description of Artificial Sequence: Synthetic HDR
tccatgtcgatcctgatgct

SEQ ID NO: 108 - DNA, 20 bp, Artificial Sequence
  Description of Artificial Sequence: Synthetic HDR
tccatgtcgctcctgatgct

SEQ ID NO: 109 - DNA, 20 bp, Artificial Sequence
  Description of Artificial Sequence: Synthetic HDR
tccatgtcgttcctgatgct

SEQ ID NO: 110 - DNA, 20 bp, Artificial Sequence
  Description of Artificial Sequence: Synthetic HDR
tccataacgttcctgatgct

SEQ ID NO: 111 - DNA, 20 bp, Artificial Sequence
  Description of Artificial Sequence: Synthetic HDR
tccatgacgtccctgatgct

SEQ ID NO: 112 - DNA, 15 bp, Artificial Sequence
  Description of Artificial Sequence: Synthetic HDR
gctagacgttagcgt

SEQ ID NO: 113 - DNA, 8 bp, Artificial Sequence
  Description of Artificial Sequence: Synthetic HDR
tcaacgtt

SEQ ID NO: 114 - DNA, 20 bp, Artificial Sequence
  Description of Artificial Sequence: Synthetic HDR
tccatgacgttcctgatgct

SEQ ID NO: 115 - DNA, 20 bp, Artificial Sequence
  Description of Artificial Sequence: Synthetic HDR
atcgactctcgagcgttctc

SEQ ID NO: 116 - DNA, 15 bp, Artificial Sequence
  Description of Artificial Sequence: Synthetic HDR
gcatgacgttgagct

SEQ ID NO: 117 - DNA, 8 bp, Artificial Sequence
  Description of Artificial Sequence: Synthetic HDR
tcagcgct

SEQ ID NO: 118 - DNA, 20 bp, Artificial Sequence
  Description of Artificial Sequence: Synthetic HDR
gagaacgctggaccttccat

SEQ ID NO: 119 - DNA, 20 bp, Artificial Sequence
  Description of Artificial Sequence: Synthetic HDR
gagaacgctcgaccttccat

SEQ ID NO: 120 - DNA, 20 bp, Artificial Sequence
  Description of Artificial Sequence: Synthetic HDR
gagaacgctcgaccttcgat

SEQ ID NO: 121 - DNA, 20 bp, Artificial Sequence
  Description of Artificial Sequence: Synthetic HDR
gagaacgctccagcactgat

SEQ ID NO: 122 - DNA, 20 bp, Artificial Sequence
  Description of Artificial Sequence: Synthetic HDR
gagaacgctccagcactgat

SEQ ID NO: 123 - DNA, 20 bp, Artificial Sequence
  Description of Artificial Sequence: Synthetic HDR
gagaacgctccagcactgat

SEQ ID NO: 124 - DNA, 20 bp, Artificial Sequence
  Description of Artificial Sequence: Synthetic HDR
tccatgtcggtcctgatgct

SEQ ID NO: 125 - DNA, 20 bp, Artificial Sequence
  Description of Artificial Sequence: Synthetic HDR
tccatgtcggtcctgctgat

SEQ ID NO: 126 - DNA, 20 bp, Artificial Sequence
  Description of Artificial Sequence: Synthetic HDR
atggactctccagcgttctc

SEQ ID NO: 127 - DNA, 20 bp, Artificial Sequence
  Description of Artificial Sequence: Synthetic HDR
atggaaggtccaacgttctc

SEQ ID NO: 128 - DNA, 20 bp, Artificial Sequence
  Description of Artificial Sequence: Synthetic HDR
tccatggcggtcctgatgct

SEQ ID NO: 129 - DNA, 20 bp, Artificial Sequence
  Description of Artificial Sequence: Synthetic HDR
tccatgacggtcctgatgct

SEQ ID NO: 130 - DNA, 20 bp, Artificial Sequence
  Description of Artificial Sequence: Synthetic HDR
tccatgtcgatcctgatgct

SEQ ID NO: 131 - DNA, 20 bp, Artificial Sequence
  Description of Artificial Sequence: Synthetic HDR
tccatgtcgctcctgatgct

SEQ ID NO: 132 - DNA, 20 bp, Artificial Sequence
  Description of Artificial Sequence: Synthetic HDR
tccatgtcgttcctgatgct

SEQ ID NO: 133 - DNA, 20 bp, Artificial Sequence
  Description of Artificial Sequence: Synthetic HDR
tccataacgttcctgatgct

SEQ ID NO: 134 - DNA, 20 bp, Artificial Sequence
  Description of Artificial Sequence: Synthetic HDR
tccatgacgtccctgatgct

SEQ ID NO: 135 - DNA, 15 bp, Artificial Sequence
  Description of Artificial Sequence: Synthetic HDR
gctagacgttagcgt

SEQ ID NO: 136 - DNA, 18 bp, Artificial Sequence
  Description of Artificial Sequence: Synthetic HDR
tcaacgtttcacgtaaaa

SEQ ID NO: 137 - DNA, 18 bp, Artificial Sequence
  Description of Artificial Sequence: Synthetic HDR
aaaatcaacgtttcacgt

SEQ ID NO: 138 - DNA, 20 bp, Artificial Sequence
  Description of Artificial Sequence: Synthetic HDR
tccatgacgttcctgatgct

SEQ ID NO: 139 - DNA, 20 bp, Artificial Sequence
  Description of Artificial Sequence: Synthetic HDR
atcgactctcgagcgttctc

SEQ ID NO: 140 - DNA, 15 bp, Artificial Sequence
  Description of Artificial Sequence: Synthetic HDR
gcatgacgttgagct

SEQ ID NO: 141 - DNA, 8 bp, Artificial Sequence
  Description of Artificial Sequence: Synthetic HDR
tcagcgct

SEQ ID NO: 142 - DNA, 8 bp, Artificial Sequence
  Description of Artificial Sequence: Synthetic HDR
tcagcgct

SEQ ID NO: 143 - DNA, 20 bp, Artificial Sequence
  Description of Artificial Sequence: Synthetic HDR
gagaacgctggaccttccat

SEQ ID NO: 144 - DNA, 20 bp, Artificial Sequence
  Description of Artificial Sequence: Synthetic HDR
gagaacgctcgaccttccat

SEQ ID NO: 145 - DNA, 20 bp, Artificial Sequence
  Description of Artificial Sequence: Synthetic HDR
gagaacgctcgaccttcgat

SEQ ID NO: 146 - DNA, 20 bp, Artificial Sequence
  Description of Artificial Sequence: Synthetic HDR
gagaacgctccagcactgat

SEQ ID NO: 147 - DNA, 20 bp, Artificial Sequence
  Description of Artificial Sequence: Synthetic HDR
gagaacgctccagcactgat

SEQ ID NO: 148 - DNA, 20 bp, Artificial Sequence
  Description of Artificial Sequence: Synthetic HDR
gagaacgctccagcactgat

SEQ ID NO: 149 - DNA, 20 bp, Artificial Sequence
  Description of Artificial Sequence: Synthetic HDR
gagaacgctccagcactgat

SEQ ID NO: 150 - DNA, 26 bp, Artificial Sequence
  Description of Artificial Sequence: Synthetic HDR
gagaacgctccagcactgattttttt

SEQ ID NO: 151 - DNA, 25 bp, Artificial Sequence
  Description of Artificial Sequence: Synthetic HDR
gagaacgctccagcactgaaaaaaa

SEQ ID NO: 152 - DNA, 20 bp, Artificial Sequence
  Description of Artificial Sequence: Synthetic HDR
tccatgtcggtcctgatgct

SEQ ID NO: 153 - DNA, 20 bp, Artificial Sequence
  Description of Artificial Sequence: Synthetic HDR
tccatgtcggtcctgctgat

SEQ ID NO: 154 - DNA, 20 bp, Artificial Sequence
  Description of Artificial Sequence: Synthetic HDR
atggactctccagcgttctc

SEQ ID NO: 155 - DNA, 20 bp, Artificial Sequence
  Description of Artificial Sequence: Synthetic HDR
atggaaggtccaacgttctc

SEQ ID NO: 156 - DNA, 20 bp, Artificial Sequence
  Description of Artificial Sequence: Synthetic HDR
tccatggcggtcctgatgct

SEQ ID NO: 157 - DNA, 20 bp, Artificial Sequence
  Description of Artificial Sequence: Synthetic HDR
tccatgacggtcctgatgct

SEQ ID NO: 158 - DNA, 20 bp, Artificial Sequence
  Description of Artificial Sequence: Synthetic HDR
tccatgtcgatcctgatgct

SEQ ID NO: 159 - DNA, 20 bp, Artificial Sequence
  Description of Artificial Sequence: Synthetic HDR
tccatgtcgctcctgatgct

SEQ ID NO: 160 - DNA, 20 bp, Artificial Sequence
  Description of Artificial Sequence: Synthetic HDR
tccatgtcgttcctgatgct

SEQ ID NO: 161 - DNA, 20 bp, Artificial Sequence
  Description of Artificial Sequence: Synthetic HDR
tccataacgttcctgatgct

SEQ ID NO: 162 - DNA, 20 bp, Artificial Sequence
  Description of Artificial Sequence: Synthetic HDR
tccatgacgtccctgatgct

SEQ ID NO: 163 - DNA, 15 bp, Artificial Sequence
  Description of Artificial Sequence: Synthetic HDR
gctagacgttagcgt

SEQ ID NO: 164 - DNA, 8 bp, Artificial Sequence
  Description of Artificial Sequence: Synthetic HDR
tcaacgtt

SEQ ID NO: 165 - DNA, 20 bp, Artificial Sequence
  Description of Artificial Sequence: Synthetic HDR
tccatgacgttcctgatgct

SEQ ID NO: 166 - DNA, 20 bp, Artificial Sequence
  Description of Artificial Sequence: Synthetic HDR
atcgactctcgagcgttctc

SEQ ID NO: 167 - DNA, 15 bp, Artificial Sequence
  Description of Artificial Sequence: Synthetic HDR
gcatgacgttgagct

SEQ ID NO: 168 - DNA, 8 bp, Artificial Sequence
  Description of Artificial Sequence: Synthetic HDR
tcagcgct

SEQ ID NO: 169 - DNA, 20 bp, Artificial Sequence
  Description of Artificial Sequence: Synthetic HDR
gagaacgctggaccttccat

SEQ ID NO: 170 - DNA, 20 bp, Artificial Sequence
  Description of Artificial Sequence: Synthetic HDR
gagaacgctcgaccttccat

SEQ ID NO: 171 - DNA, 20 bp, Artificial Sequence
  Description of Artificial Sequence: Synthetic HDR
gagaacgctcgaccttcgat

SEQ ID NO: 172 - DNA, 20 bp, Artificial Sequence
  Description of Artificial Sequence: Synthetic HDR
gagaacgctccagcactgat

SEQ ID NO: 173 - DNA, 20 bp, Artificial Sequence
  Description of Artificial Sequence: Synthetic HDR
tccatgtcggtcctgatgct

SEQ ID NO: 174 - DNA, 20 bp, Artificial Sequence
  Description of Artificial Sequence: Synthetic HDR
tccatgtcggtcctgctgat

SEQ ID NO: 175 - DNA, 20 bp, Artificial Sequence
  Description of Artificial Sequence: Synthetic HDR
atggactctccagcgttctc

SEQ ID NO: 176 - DNA, 20 bp, Artificial Sequence
  Description of Artificial Sequence: Synthetic HDR
atggaaggtccaacgttctc

SEQ ID NO: 177 - DNA, 20 bp, Artificial Sequence
  Description of Artificial Sequence: Synthetic HDR
tccatggcggtcctgatgct

SEQ ID NO: 178 - DNA, 20 bp, Artificial Sequence
  Description of Artificial Sequence: Synthetic HDR
tccatgacggtcctgatgct

SEQ ID NO: 179 - DNA, 20 bp, Artificial Sequence
  Description of Artificial Sequence: Synthetic HDR
tccatgtcgatcctgatgct

SEQ ID NO: 180 - DNA, 20 bp, Artificial Sequence
  Description of Artificial Sequence: Synthetic HDR
tccatgtcgctcctgatgct

SEQ ID NO: 181 - DNA, 20 bp, Artificial Sequence
  Description of Artificial Sequence: Synthetic HDR
tccatgtcgttcctgatgct

SEQ ID NO: 182 - DNA, 20 bp, Artificial Sequence
  Description of Artificial Sequence: Synthetic HDR
tccataacgttcctgatgct

SEQ ID NO: 183 - DNA, 20 bp, Artificial Sequence
  Description of Artificial Sequence: Synthetic HDR
tccatgacgtccctgatgct

SEQ ID NO: 184 - DNA, 15 bp, Artificial Sequence
  Description of Artificial Sequence: Synthetic HDR
gctagacgttagcgt

SEQ ID NO: 185 - DNA, 8 bp, Artificial Sequence
  Description of Artificial Sequence: Synthetic HDR
tcaacgtt

SEQ ID NO: 186 - DNA, 20 bp, Artificial Sequence
  Description of Artificial Sequence: Synthetic HDR
tccatgacgttcctgatgct

SEQ ID NO: 187 - DNA, 20 bp, Artificial Sequence
  Description of Artificial Sequence: Synthetic HDR
atcgactctcgagcgttctc

SEQ ID NO: 188 - DNA, 15 bp, Artificial Sequence
  Description of Artificial Sequence: Synthetic HDR
gcatgacgttgagct

SEQ ID NO: 189 - DNA, 8 bp, Artificial Sequence
  Description of Artificial Sequence: Synthetic HDR
tcagcgct

SEQ ID NO: 190 - DNA, 20 bp, Artificial Sequence
  Description of Artificial Sequence: Synthetic HDR
gagaacgctggaccttccat

SEQ ID NO: 191 - DNA, 20 bp, Artificial Sequence
  Description of Artificial Sequence: Synthetic HDR
gagaacgctcgaccttccat

SEQ ID NO: 192 - DNA, 20 bp, Artificial Sequence
  Description of Artificial Sequence: Synthetic HDR
gagaacgctcgaccttccat

SEQ ID NO: 193 - DNA, 20 bp, Artificial Sequence
  Description of Artificial Sequence: Synthetic HDR
gagaacgctcgaccttccat

SEQ ID NO: 194 - DNA, 20 bp, Artificial Sequence
  Description of Artificial Sequence: Synthetic HDR
gagaacgctcgaccttcgat

SEQ ID NO: 195 - DNA, 20 bp, Artificial Sequence
  Description of Artificial Sequence: Synthetic HDR
gagaacgctcgaccttcgat

SEQ ID NO: 196 - DNA, 20 bp, Artificial Sequence
  Description of Artificial Sequence: Synthetic HDR
gagaacgctcgaccttcgat

SEQ ID NO: 197 - DNA, 20 bp, Artificial Sequence
  Description of Artificial Sequence: Synthetic HDR
gagaacgctccagcactgat

SEQ ID NO: 198 - DNA, 20 bp, Artificial Sequence
  Description of Artificial Sequence: Synthetic HDR
tccatgtcggtcctgatgct

SEQ ID NO: 199 - DNA, 20 bp, Artificial Sequence
  Description of Artificial Sequence: Synthetic HDR
tccatgtcggtcctgctgat

SEQ ID NO: 200 - DNA, 20 bp, Artificial Sequence
  Description of Artificial Sequence: Synthetic HDR
atggactctccagcgttctc

SEQ ID NO: 201 - DNA, 20 bp, Artificial Sequence
  Description of Artificial Sequence: Synthetic HDR
atggaaggtccaacgttctc

SEQ ID NO: 202 - DNA, 20 bp, Artificial Sequence
  Description of Artificial Sequence: Synthetic HDR
tccatggcggtcctgatgct

SEQ ID NO: 203 - DNA, 20 bp, Artificial Sequence
  Description of Artificial Sequence: Synthetic HDR
tccatgacggtcctgatgct

SEQ ID NO: 204 - DNA, 20 bp, Artificial Sequence
  Description of Artificial Sequence: Synthetic HDR
tccatgtcgatcctgatgct

SEQ ID NO: 205 - DNA, 20 bp, Artificial Sequence
  Description of Artificial Sequence: Synthetic HDR
tccatgtcgctcctgatgct

SEQ ID NO: 206 - DNA, 20 bp, Artificial Sequence
  Description of Artificial Sequence: Synthetic HDR
tccatgtcgttcctgatgct

SEQ ID NO: 207 - DNA, 20 bp, Artificial Sequence
  Description of Artificial Sequence: Synthetic HDR
tccataacgttcctgatgct

SEQ ID NO: 208 - DNA, 20 bp, Artificial Sequence
  Description of Artificial Sequence: Synthetic HDR
tccatgacgtccctgatgct

SEQ ID NO: 209 - DNA, 20 bp, Artificial Sequence
  Description of Artificial Sequence: Synthetic HDR
gctagacgttagcgtttttt

SEQ ID NO: 210 - DNA, 13 bp, Artificial Sequence
  Description of Artificial Sequence: Synthetic HDR
tcaacgttttttt

SEQ ID NO: 211 - DNA, 25 bp, Artificial Sequence
  Description of Artificial Sequence: Synthetic HDR
tccatgacgttcctgatgctttttt

SEQ ID NO: 212 - DNA, 25 bp, Artificial Sequence
  Description of Artificial Sequence: Synthetic HDR
atcgactctcgagcgttctcttttt

SEQ ID NO: 213 - DNA, 20 bp, Artificial Sequence
  Description of Artificial Sequence: Synthetic HDR
gcatgacgttgagctttttt

SEQ ID NO: 214 - DNA, 13 bp, Artificial Sequence
  Description of Artificial Sequence: Synthetic HDR
tcagcgctttttt

SEQ ID NO: 215 - DNA, 25 bp, Artificial Sequence
  Description of Artificial Sequence: Synthetic HDR
gagaacgctggaccttccatttttt

SEQ ID NO: 216 - DNA, 25 bp, Artificial Sequence
  Description of Artificial Sequence: Synthetic HDR
gagaacgctcgaccttccatttttt

SEQ ID NO: 217 - DNA, 25 bp, Artificial Sequence
  Description of Artificial Sequence: Synthetic HDR
gagaacgctcgaccttcgatttttt

SEQ ID NO: 218 - DNA, 24 bp, Artificial Sequence
  Description of Artificial Sequence: Synthetic HDR
gagaacgctccagcactgattttt

SEQ ID NO: 219 - DNA, 27 bp, Artificial Sequence
  Description of Artificial Sequence: Synthetic HDR
tccatgtcggtcctgatgctttttttt

SEQ ID NO: 220 - DNA, 24 bp, Artificial Sequence
  Description of Artificial Sequence: Synthetic HDR
tccatgtcggtcctgctgattttt

SEQ ID NO: 221 - DNA, 25 bp, Artificial Sequence
  Description of Artificial Sequence: Synthetic HDR
atggactctccagcgttctcttttt

SEQ ID NO: 222 - DNA, 25 bp, Artificial Sequence
  Description of Artificial Sequence: Synthetic HDR
atggaaggtccaacgttctcttttt

SEQ ID NO: 223 - DNA, 25 bp, Artificial Sequence
  Description of Artificial Sequence: Synthetic HDR
tccatggcggtcctgatgctttttt

SEQ ID NO: 224 - DNA, 25 bp, Artificial Sequence
  Description of Artificial Sequence: Synthetic HDR
tccatgacggtcctgatgctttttt

SEQ ID NO: 225 - DNA, 25 bp, Artificial Sequence
  Description of Artificial Sequence: Synthetic HDR
tccatgtcgatcctgatgctttttt

SEQ ID NO: 226 - DNA, 25 bp, Artificial Sequence
  Description of Artificial Sequence: Synthetic HDR
tccatgtcgctcctgatgctttttt

SEQ ID NO: 227 - DNA, 24 bp, Artificial Sequence
  Description of Artificial Sequence: Synthetic HDR
tccatgtcgttcctgatgcttttt

SEQ ID NO: 228 - DNA, 24 bp, Artificial Sequence
  Description of Artificial Sequence: Synthetic HDR
tccataacgttcctgatgcttttt

SEQ ID NO: 229 - DNA, 24 bp, Artificial Sequence
  Description of Artificial Sequence: Synthetic HDR
tccatgacgtccctgatgcttttt

SEQ ID NO: 230 - DNA, 23 bp, Artificial Sequence
  Description of Artificial Sequence: Synthetic HDR
atatatatgctagacgttagcgt

SEQ ID NO: 231 - DNA, 15 bp, Artificial Sequence
  Description of Artificial Sequence: Synthetic HDR
atatatatcaacgtt

SEQ ID NO: 232 - DNA, 27 bp, Artificial Sequence
  Description of Artificial Sequence: Synthetic HDR
atatatatccatgacgttcctgatgct

SEQ ID NO: 233 - DNA, 26 bp, Artificial Sequence
  Description of Artificial Sequence: Synthetic HDR
atatatatcgactctcgagcgttctc

SEQ ID NO: 234 - DNA, 23 bp, Artificial Sequence
  Description of Artificial Sequence: Synthetic HDR
atatatatgcatgacgttgagct

SEQ ID NO: 235 - DNA, 15 bp, Artificial Sequence
  Description of Artificial Sequence: Synthetic HDR
atatatatcagcgct

SEQ ID NO: 236 - DNA, 28 bp, Artificial Sequence
  Description of Artificial Sequence: Synthetic HDR
atatatatgagaacgctggaccttccat

SEQ ID NO: 237 - DNA, 30 bp, Artificial Sequence
  Description of Artificial Sequence: Synthetic HDR
atatatatatgagaacgctcgaccttccat

SEQ ID NO: 238 - DNA, 28 bp, Artificial Sequence
  Description of Artificial Sequence: Synthetic HDR
atatatatgagaacgctcgaccttcgat

SEQ ID NO: 239 - DNA, 28 bp, Artificial Sequence
  Description of Artificial Sequence: Synthetic HDR
atatatatgagaacgctccagcactgat

SEQ ID NO: 240 - DNA, 28 bp, Artificial Sequence
  Description of Artificial Sequence: Synthetic HDR
atatatattccatgtcggtcctgatgct

SEQ ID NO: 241 - DNA, 28 bp, Artificial Sequence
  Description of Artificial Sequence: Synthetic HDR
atatatattccatgtcggtcctgctgat

SEQ ID NO: 242 - DNA, 28 bp, Artificial Sequence
  Description of Artificial Sequence: Synthetic HDR
atatatatatggactctccagcgttctc

SEQ ID NO: 243 - DNA, 28 bp, Artificial Sequence
  Description of Artificial Sequence: Synthetic HDR
atatatatatggaaggtccaacgttctc

SEQ ID NO: 244 - DNA, 28 bp, Artificial Sequence
  Description of Artificial Sequence: Synthetic HDR
atatatattccatggcggtcctgatgct

SEQ ID NO: 245 - DNA, 28 bp, Artificial Sequence
  Description of Artificial Sequence: Synthetic HDR
atatatattccatgacggtcctgatgct

SEQ ID NO: 246 - DNA, 28 bp, Artificial Sequence
  Description of Artificial Sequence: Synthetic HDR
atatatattccatgtcgatcctgatgct

SEQ ID NO: 247 - DNA, 28 bp, Artificial Sequence
  Description of Artificial Sequence: Synthetic HDR
atatatattccatgtcgctcctgatgct

SEQ ID NO: 248 - DNA, 28 bp, Artificial Sequence
  Description of Artificial Sequence: Synthetic HDR
atatatattccatgtcgttcctgatgct

SEQ ID NO: 249 - DNA, 28 bp, Artificial Sequence
  Description of Artificial Sequence: Synthetic HDR
atatatattccataacgttcctgatgct

SEQ ID NO: 250 - DNA, 27 bp, Artificial Sequence
  Description of Artificial Sequence: Synthetic HDR
atatatatccatgacgtccctgatgct

SEQ ID NO: 251 - DNA, 29 bp, Artificial Sequence
  Description of Artificial Sequence: Synthetic HDR
aaaaaaagctagacgttagcgtttttttt

SEQ ID NO: 252 - DNA, 22 bp, Artificial Sequence
  Description of Artificial Sequence: Synthetic HDR
aaaaaaatcaacgttttttttt

SEQ ID NO: 253 - DNA, 34 bp, Artificial Sequence
  Description of Artificial Sequence: Synthetic HDR
aaaaaaatccatgacgttcctgatgctttttttt

SEQ ID NO: 254 - DNA, 34 bp, Artificial Sequence
  Description of Artificial Sequence: Synthetic HDR
aaaaaaaatcgactctcgagcgttctcttttttt

SEQ ID NO: 255 - DNA, 29 bp, Artificial Sequence
  Description of Artificial Sequence: Synthetic HDR
aaaaaaagcatgacgttgagctttttttt

SEQ ID NO: 256 - DNA, 22 bp, Artificial Sequence
  Description of Artificial Sequence: Synthetic HDR
aaaaaaatcagcgctttttttt

SEQ ID NO: 257 - DNA, 34 bp, Artificial Sequence
  Description of Artificial Sequence: Synthetic HDR
aaaaaaagagaacgctggaccttccatttttttt

SEQ ID NO: 258 - DNA, 34 bp, Artificial Sequence
  Description of Artificial Sequence: Synthetic HDR
aaaaaaagagaacgctcgaccttccatttttttt

SEQ ID NO: 259 - DNA, 34 bp, Artificial Sequence
  Description of Artificial Sequence: Synthetic HDR
aaaaaaagagaacgctcgaccttcgatttttttt

SEQ ID NO: 260 - DNA, 34 bp, Artificial Sequence
  Description of Artificial Sequence: Synthetic HDR
aaaaaaagagaacgctccagcactgatttttttt

SEQ ID NO: 261 - DNA, 34 bp, Artificial Sequence
  Description of Artificial Sequence: Synthetic HDR
aaaaaaatccatgtcggtcctgatgctttttttt

SEQ ID NO: 262 - DNA, 34 bp, Artificial Sequence
  Description of Artificial Sequence: Synthetic HDR
aaaaaaatccatgtcggtcctgctgatttttttt

SEQ ID NO: 263 - DNA, 34 bp, Artificial Sequence
  Description of Artificial Sequence: Synthetic HDR
aaaaaaaatggactctccagcgttctcttttttt

SEQ ID NO: 264 - DNA, 34 bp, Artificial Sequence
  Description of Artificial Sequence: Synthetic HDR
aaaaaaaatggaaggtccaacgttctcttttttt

SEQ ID NO: 265 - DNA, 34 bp, Artificial Sequence
  Description of Artificial Sequence: Synthetic HDR
aaaaaaatccatggcggtcctgatgctttttttt

SEQ ID NO: 266 - DNA, 34 bp, Artificial Sequence
  Description of Artificial Sequence: Synthetic HDR
aaaaaaatccatgacggtcctgatgctttttttt

SEQ ID NO: 267 - DNA, 34 bp, Artificial Sequence
  Description of Artificial Sequence: Synthetic HDR
aaaaaaatccatgtcgatcctgatgctttttttt

SEQ ID NO: 268 - DNA, 34 bp, Artificial Sequence
  Description of Artificial Sequence: Synthetic HDR
aaaaaaatccatgtcgctcctgatgctttttttt

SEQ ID NO: 269 - DNA, 34 bp, Artificial Sequence
  Description of Artificial Sequence: Synthetic HDR
aaaaaaatccatgtcgttcctgatgctttttttt

SEQ ID NO: 270 - DNA, 28 bp, Artificial Sequence
  Description of Artificial Sequence: Synthetic HDR
aaaaaaagctagacgttagcgttttttt

SEQ ID NO: 271 - DNA, 19 bp, Artificial Sequence
  Description of Artificial Sequence: Synthetic HDR
aaaaaaacaacgttttttt

SEQ ID NO: 272 - DNA, 33 bp, Artificial Sequence
  Description of Artificial Sequence: Synthetic HDR
aaaaaaatccatgacgttcctgatgcttttttt

SEQ ID NO: 273 - DNA, 33 bp, Artificial Sequence
  Description of Artificial Sequence: Synthetic HDR
aaaaaaatcgactctcgagcgttctcttttttt

SEQ ID NO: 274 - DNA, 28 bp, Artificial Sequence
  Description of Artificial Sequence: Synthetic HDR
aaaaaaagcatgacgttgagcttttttt

SEQ ID NO: 275 - DNA, 21 bp, Artificial Sequence
  Description of Artificial Sequence: Synthetic HDR
aaaaaaatcagcgcttttttt

SEQ ID NO: 276 - DNA, 33 bp, Artificial Sequence
  Description of Artificial Sequence: Synthetic HDR
aaaaaaagagaacgctggaccttccattttttt

SEQ ID NO: 277 - DNA, 33 bp, Artificial Sequence
  Description of Artificial Sequence: Synthetic HDR
aaaaaaagagaacgctcgaccttccattttttt

SEQ ID NO: 278 - DNA, 33 bp, Artificial Sequence
  Description of Artificial Sequence: Synthetic HDR
aaaaaaagagaacgctcgaccttcgattttttt

SEQ ID NO: 279 - DNA, 33 bp, Artificial Sequence
  Description of Artificial Sequence: Synthetic HDR
aaaaaaagagaacgctccagcactgattttttt

SEQ ID NO: 280 - DNA, 33 bp, Artificial Sequence
  Description of Artificial Sequence: Synthetic HDR
aaaaaaatccatgtcggtcctgatgcttttttt

SEQ ID NO: 281 - DNA, 33 bp, Artificial Sequence
  Description of Artificial Sequence: Synthetic HDR
aaaaaaatccatgtcggtcctgctgattttttt

SEQ ID NO: 282 - DNA, 33 bp, Artificial Sequence
  Description of Artificial Sequence: Synthetic HDR
aaaaaaatggactctccagcgttctcttttttt

SEQ ID NO: 283 - DNA, 33 bp, Artificial Sequence
  Description of Artificial Sequence: Synthetic HDR
aaaaaaatggaaggtccaacgttctcttttttt

SEQ ID NO: 284 - DNA, 33 bp, Artificial Sequence
  Description of Artificial Sequence: Synthetic HDR
aaaaaaatccatggcggtcctgatgcttttttt

SEQ ID NO: 285 - DNA, 33 bp, Artificial Sequence
  Description of Artificial Sequence: Synthetic HDR
aaaaaaatccatgacggtcctgatgcttttttt

SEQ ID NO: 286 - DNA, 33 bp, Artificial Sequence
  Description of Artificial Sequence: Synthetic HDR
aaaaaaatccatgtcgatcctgatgcttttttt

SEQ ID NO: 287 - DNA, 33 bp, Artificial Sequence
  Description of Artificial Sequence: Synthetic HDR
aaaaaaatccatgtcgctcctgatgcttttttt

SEQ ID NO: 288 - DNA, 33 bp, Artificial Sequence
  Description of Artificial Sequence: Synthetic HDR
aaaaaaatccatgtcgttcctgatgcttttttt

SEQ ID NO: 289 - DNA, 16 bp, Artificial Sequence
  Description of Artificial Sequence: Synthetic HDR
agctagacgttagcgt

SEQ ID NO: 290 - DNA, 9 bp, Artificial Sequence
  Description of Artificial Sequence: Synthetic HDR
atcaacgtt

SEQ ID NO: 291 - DNA, 21 bp, Artificial Sequence
  Description of Artificial Sequence: Synthetic HDR
atccatgacgttcctgatgct

SEQ ID NO: 292 - DNA, 21 bp, Artificial Sequence
  Description of Artificial Sequence: Synthetic HDR
aatcgactctcgagcgttctc

SEQ ID NO: 293 - DNA, 16 bp, Artificial Sequence
  Description of Artificial Sequence: Synthetic HDR
agcatgacgttgagct

SEQ ID NO: 294 - DNA, 9 bp, Artificial Sequence
  Description of Artificial Sequence: Synthetic HDR
atcagcgct

SEQ ID NO: 295 - DNA, 21 bp, Artificial Sequence
  Description of Artificial Sequence: Synthetic HDR
agagaacgctggaccttccat

SEQ ID NO: 296 - DNA, 21 bp, Artificial Sequence
  Description of Artificial Sequence: Synthetic HDR
agagaacgctcgaccttccat

SEQ ID NO: 297 - DNA, 21 bp, Artificial Sequence
  Description of Artificial Sequence: Synthetic HDR
agagaacgctcgaccttcgat

SEQ ID NO: 298 - DNA, 21 bp, Artificial Sequence
  Description of Artificial Sequence: Synthetic HDR
agagaacgctccagcactgat

SEQ ID NO: 299 - DNA, 21 bp, Artificial Sequence
  Description of Artificial Sequence: Synthetic HDR
atccatgtcggtcctgatgct

SEQ ID NO: 300 - DNA, 21 bp, Artificial Sequence
  Description of Artificial Sequence: Synthetic HDR
atccatgtcggtcctgctgat

SEQ ID NO: 301 - DNA, 21 bp, Artificial Sequence
  Description of Artificial Sequence: Synthetic HDR
aatggactctccagcgttctc

SEQ ID NO: 302 - DNA, 21 bp, Artificial Sequence
  Description of Artificial Sequence: Synthetic HDR
aatggaaggtccaacgttctc

SEQ ID NO: 303 - DNA, 21 bp, Artificial Sequence
  Description of Artificial Sequence: Synthetic HDR
atccatggcggtcctgatgct

SEQ ID NO: 304 - DNA, 21 bp, Artificial Sequence
  Description of Artificial Sequence: Synthetic HDR
atccatgacggtcctgatgct

SEQ ID NO: 305 - DNA, 21 bp, Artificial Sequence
  Description of Artificial Sequence: Synthetic HDR
atccatgtcgatcctgatgct

SEQ ID NO: 306 - DNA, 21 bp, Artificial Sequence
  Description of Artificial Sequence: Synthetic HDR
atccatgtcgctcctgatgct

SEQ ID NO: 307 - DNA, 21 bp, Artificial Sequence
  Description of Artificial Sequence: Synthetic HDR
atccatgtcgttcctgatgct

SEQ ID NO: 308 - DNA, 16 bp, Artificial Sequence
  Description of Artificial Sequence: Synthetic HDR
gctagacgttagcgta

SEQ ID NO: 309 - DNA, 9 bp, Artificial Sequence
  Description of Artificial Sequence: Synthetic HDR
tcaacgtta

SEQ ID NO: 310 - DNA, 21 bp, Artificial Sequence
  Description of Artificial Sequence: Synthetic HDR
tccatgacgttcctgatgcta

SEQ ID NO: 311 - DNA, 21 bp, Artificial Sequence
  Description of Artificial Sequence: Synthetic HDR
atcgactctcgagcgttctca

SEQ ID NO: 312 - DNA, 16 bp, Artificial Sequence
  Description of Artificial Sequence: Synthetic HDR
gcatgacgttgagcta

SEQ ID NO: 313 - DNA, 9 bp, Artificial Sequence
  Description of Artificial Sequence: Synthetic HDR
tcagcgcta

SEQ ID NO: 314 - DNA, 21 bp, Artificial Sequence
  Description of Artificial Sequence: Synthetic HDR
gagaacgctggaccttccata

SEQ ID NO: 315 - DNA, 21 bp, Artificial Sequence
  Description of Artificial Sequence: Synthetic HDR
gagaacgctcgaccttccata

SEQ ID NO: 316 - DNA, 21 bp, Artificial Sequence
  Description of Artificial Sequence: Synthetic HDR
gagaacgctcgaccttcgata

SEQ ID NO: 317 - DNA, 21 bp, Artificial Sequence
  Description of Artificial Sequence: Synthetic HDR
gagaacgctccagcactgata

SEQ ID NO: 318 - DNA, 21 bp, Artificial Sequence
  Description of Artificial Sequence: Synthetic HDR
tccatgtcggtcctgatgcta

SEQ ID NO: 319 - DNA, 21 bp, Artificial Sequence
  Description of Artificial Sequence: Synthetic HDR
tccatgtcggtcctgctgata

SEQ ID NO: 320 - DNA, 21 bp, Artificial Sequence
  Description of Artificial Sequence: Synthetic HDR
atggactctccagcgttctca

SEQ ID NO: 321 - DNA, 21 bp, Artificial Sequence
  Description of Artificial Sequence: Synthetic HDR
atggaaggtccaacgttctca

SEQ ID NO: 322 - DNA, 21 bp, Artificial Sequence
  Description of Artificial Sequence: Synthetic HDR
tccatggcggtcctgatgcta

SEQ ID NO: 323 - DNA, 21 bp, Artificial Sequence
  Description of Artificial Sequence: Synthetic HDR
tccatgacggtcctgatgcta

SEQ ID NO: 324 - DNA, 21 bp, Artificial Sequence
  Description of Artificial Sequence: Synthetic HDR
tccatgtcgatcctgatgcta

SEQ ID NO: 325 - DNA, 21 bp, Artificial Sequence
  Description of Artificial Sequence: Synthetic HDR
tccatgtcgctcctgatgcta

SEQ ID NO: 326 - DNA, 21 bp, Artificial Sequence
  Description of Artificial Sequence: Synthetic HDR
tccatgtcgttcctgatgcta

SEQ ID NO: 327 - DNA, 17 bp, Artificial Sequence
  Description of Artificial Sequence: Synthetic HDR
agctagacgttagcgta

SEQ ID NO: 328 - DNA, 10 bp, Artificial Sequence
  Description of Artificial Sequence: Synthetic HDR
atcaacgtta

SEQ ID NO: 329 - DNA, 22 bp, Artificial Sequence
  Description of Artificial Sequence: Synthetic HDR
atccatgacgttcctgatgcta

SEQ ID NO: 330 - DNA, 22 bp, Artificial Sequence
  Description of Artificial Sequence: Synthetic HDR
aatcgactctcgagcgttctca

SEQ ID NO: 331 - DNA, 17 bp, Artificial Sequence
  Description of Artificial Sequence: Synthetic HDR
agcatgacgttgagcta

SEQ ID NO: 332 - DNA, 10 bp, Artificial Sequence
  Description of Artificial Sequence: Synthetic HDR
atcagcgcta

SEQ ID NO: 333 - DNA, 22 bp, Artificial Sequence
  Description of Artificial Sequence: Synthetic HDR
agagaacgctggaccttccata

SEQ ID NO: 334 - DNA, 22 bp, Artificial Sequence
  Description of Artificial Sequence: Synthetic HDR
agagaacgctcgaccttccata

SEQ ID NO: 335 - DNA, 22 bp, Artificial Sequence
  Description of Artificial Sequence: Synthetic HDR
agagaacgctcgaccttcgata

SEQ ID NO: 336 - DNA, 22 bp, Artificial Sequence
  Description of Artificial Sequence: Synthetic HDR
agagaacgctccagcactgata

SEQ ID NO: 337 - DNA, 22 bp, Artificial Sequence
  Description of Artificial Sequence: Synthetic HDR
atccatgtcggtcctgatgcta

SEQ ID NO: 338 - DNA, 22 bp, Artificial Sequence
  Description of Artificial Sequence: Synthetic HDR
atccatgtcggtcctgctgata

SEQ ID NO: 339 - DNA, 22 bp, Artificial Sequence
  Description of Artificial Sequence: Synthetic HDR
aatggactctccagcgttctca

SEQ ID NO: 340 - DNA, 22 bp, Artificial Sequence
  Description of Artificial Sequence: Synthetic HDR
aatggaaggtccaacgttctca

SEQ ID NO: 341 - DNA, 22 bp, Artificial Sequence
  Description of Artificial Sequence: Synthetic HDR
atccatggcggtcctgatgcta

SEQ ID NO: 342 - DNA, 22 bp, Artificial Sequence
  Description of Artificial Sequence: Synthetic HDR
atccatgacggtcctgatgcta

SEQ ID NO: 343 - DNA, 22 bp, Artificial Sequence
  Description of Artificial Sequence: Synthetic HDR
atccatgtcgatcctgatgcta

SEQ ID NO: 344 - DNA, 22 bp, Artificial Sequence
  Description of Artificial Sequence: Synthetic HDR
atccatgtcgctcctgatgcta

SEQ ID NO: 345 - DNA, 22 bp, Artificial Sequence
  Description of Artificial Sequence: Synthetic HDR
atccatgtcgttcctgatgcta

SEQ ID NO: 346 - DNA, 35 bp, Artificial Sequence
  Description of Artificial Sequence: Synthetic HDR
tccatgacgttcctgatgcttttttttaaaaaaaa

SEQ ID NO: 347 - DNA, 30 bp, Artificial Sequence
  Description of Artificial Sequence: Synthetic HDR
gctagacgttagcgttttttttaaaaaaaa

SEQ ID NO: 348 - DNA, 22 bp, Artificial Sequence
  Description of Artificial Sequence: Synthetic HDR
tcaacgttttttttaaaaaaaa

SEQ ID NO: 349 - DNA, 38 bp, Artificial Sequence
  Description of Artificial Sequence: Synthetic HDR
tccatgacgttcctgatgctttttttttggaaaaaaaa

SEQ ID NO: 350 - DNA, 36 bp, Artificial Sequence
  Description of Artificial Sequence: Synthetic HDR
atcgactctcgagcgttctcttttttttaaaaaaaa

SEQ ID NO: 351 - DNA, 31 bp, Artificial Sequence
  Description of Artificial Sequence: Synthetic HDR
gcatgacgttgagctttttttttaaaaaaaa

SEQ ID NO: 352 - DNA, 24 bp, Artificial Sequence
  Description of Artificial Sequence: Synthetic HDR
tcagcgctttttttttaaaaaaaa

SEQ ID NO: 353 - DNA, 36 bp, Artificial Sequence
  Description of Artificial Sequence: Synthetic HDR
gagaacgctggaccttccatttttttttaaaaaaaa

SEQ ID NO: 354 - DNA, 36 bp, Artificial Sequence
  Description of Artificial Sequence: Synthetic HDR
gagaacgctcgaccttccatttttttttaaaaaaaa

SEQ ID NO: 355 - DNA, 36 bp, Artificial Sequence
  Description of Artificial Sequence: Synthetic HDR
gagaacgctcgaccttcgatttttttttaaaaaaaa

SEQ ID NO: 356 - DNA, 37 bp, Artificial Sequence
  Description of Artificial Sequence: Synthetic HDR
gagaacgctccagcactgatttttttttaaaaaaaaa

SEQ ID NO: 357 - DNA, 37 bp, Artificial Sequence
  Description of Artificial Sequence: Synthetic HDR
tccatgtcggtcctgatgctttttttttaaaaaaaaa

SEQ ID NO: 358 - DNA, 37 bp, Artificial Sequence
  Description of Artificial Sequence: Synthetic HDR
tccatgtcggtcctgctgatttttttttaaaaaaaaa

SEQ ID NO: 359 - DNA, 36 bp, Artificial Sequence
  Description of Artificial Sequence: Synthetic HDR
atggactctccagcgttctcttttttttaaaaaaaa

SEQ ID NO: 360 - DNA, 36 bp, Artificial Sequence
  Description of Artificial Sequence: Synthetic HDR
atggaaggtccaacgttctcttttttttaaaaaaaa

SEQ ID NO: 361 - DNA, 37 bp, Artificial Sequence
  Description of Artificial Sequence: Synthetic HDR
tccatggcggtcctgatgctttttttttaaaaaaaaa

SEQ ID NO: 362 - DNA, 37 bp, Artificial Sequence
  Description of Artificial Sequence: Synthetic HDR
tccatgacggtcctgatgctttttttttaaaaaaaaa

SEQ ID NO: 363 - DNA, 37 bp, Artificial Sequence
  Description of Artificial Sequence: Synthetic HDR
tccatgtcgatcctgatgctttttttttaaaaaaaaa

SEQ ID NO: 364 - DNA, 37 bp, Artificial Sequence
  Description of Artificial Sequence: Synthetic HDR
tccatgtcgctcctgatgctttttttttaaaaaaaaa

SEQ ID NO: 365 - DNA, 37 bp, Artificial Sequence
  Description of Artificial Sequence: Synthetic HDR
tccatgtcgttcctgatgctttttttttaaaaaaaaa

SEQ ID NO: 366 - DNA, 23 bp, Artificial Sequence
  Description of Artificial Sequence: Synthetic HDR
ccccccccgctagacgttagcgt

SEQ ID NO: 367 - DNA, 16 bp, Artificial Sequence
  Description of Artificial Sequence: Synthetic HDR
cccccccctcaacgtt

SEQ ID NO: 368 - DNA, 28 bp, Artificial Sequence
  Description of Artificial Sequence: Synthetic HDR
cccccccctccatgacgttcctgatgct

SEQ ID NO: 369 - DNA, 28 bp, Artificial Sequence
  Description of Artificial Sequence: Synthetic HDR
ccccccccatcgactctcgagcgttctc

SEQ ID NO: 370 - DNA, 23 bp, Artificial Sequence
  Description of Artificial Sequence: Synthetic HDR
ccccccccgcatgacgttgagct

SEQ ID NO: 371 - DNA, 16 bp, Artificial Sequence
  Description of Artificial Sequence: Synthetic HDR
cccccccctcagcgct

SEQ ID NO: 372 - DNA, 28 bp, Artificial Sequence
  Description of Artificial Sequence: Synthetic HDR
ccccccccgagaacgctggaccttccat

SEQ ID NO: 373 - DNA, 28 bp, Artificial Sequence
  Description of Artificial Sequence: Synthetic HDR
ccccccccgagaacgctcgaccttccat

SEQ ID NO: 374 - DNA, 28 bp, Artificial Sequence
  Description of Artificial Sequence: Synthetic HDR
ccccccccgagaacgctcgaccttcgat

SEQ ID NO: 375 - DNA, 28 bp, Artificial Sequence
  Description of Artificial Sequence: Synthetic HDR
ccccccccgagaacgctccagcactgat

SEQ ID NO: 376 - DNA, 28 bp, Artificial Sequence
  Description of Artificial Sequence: Synthetic HDR
cccccccctccatgtcggtcctgatgct

SEQ ID NO: 377 - DNA, 28 bp, Artificial Sequence
  Description of Artificial Sequence: Synthetic HDR
cccccccctccatgtcggtcctgctgat

SEQ ID NO: 378 - DNA, 28 bp, Artificial Sequence
  Description of Artificial Sequence: Synthetic HDR
ccccccccatggactctccagcgttctc

SEQ ID NO: 379 - DNA, 28 bp, Artificial Sequence
  Description of Artificial Sequence: Synthetic HDR
ccccccccatggaaggtccaacgttctc

SEQ ID NO: 380 - DNA, 28 bp, Artificial Sequence
  Description of Artificial Sequence: Synthetic HDR
cccccccctccatggcggtcctgatgct

SEQ ID NO: 381 - DNA, 28 bp, Artificial Sequence
  Description of Artificial Sequence: Synthetic HDR
cccccccctccatgacggtcctgatgct

SEQ ID NO: 382 - DNA, 28 bp, Artificial Sequence
  Description of Artificial Sequence: Synthetic HDR
cccccccctccatgtcgatcctgatgct

SEQ ID NO: 383 - DNA, 28 bp, Artificial Sequence
  Description of Artificial Sequence: Synthetic HDR
cccccccctccatgtcgctcctgatgct

SEQ ID NO: 384 - DNA, 28 bp, Artificial Sequence
  Description of Artificial Sequence: Synthetic HDR
cccccccctccatgtcgttcctgatgct

SEQ ID NO: 385 - DNA, 23 bp, Artificial Sequence
  Description of Artificial Sequence: Synthetic HDR
gctagacgttagcgtgtgtgtgt

SEQ ID NO: 386 - DNA, 16 bp, Artificial Sequence
  Description of Artificial Sequence: Synthetic HDR
tcaacgttgtgtgtgt

SEQ ID NO: 387 - DNA, 28 bp, Artificial Sequence
  Description of Artificial Sequence: Synthetic HDR
tccatgacgttcctgatgctgtgtgtgt

SEQ ID NO: 388 - DNA, 28 bp, Artificial Sequence
  Description of Artificial Sequence: Synthetic HDR
atcgactctcgagcgttctcgtgtgtgt

SEQ ID NO: 389 - DNA, 23 bp, Artificial Sequence
  Description of Artificial Sequence: Synthetic HDR
gcatgacgttgagctgtgtgtgt

SEQ ID NO: 390 - DNA, 16 bp, Artificial Sequence
  Description of Artificial Sequence: Synthetic HDR
tcagcgctgtgtgtgt

SEQ ID NO: 391 - DNA, 28 bp, Artificial Sequence
  Description of Artificial Sequence: Synthetic HDR
gagaacgctggaccttccatgtgtgtgt

SEQ ID NO: 392 - DNA, 28 bp, Artificial Sequence
  Description of Artificial Sequence: Synthetic HDR
gagaacgctcgaccttccatgtgtgtgt

SEQ ID NO: 393 - DNA, 28 bp, Artificial Sequence
  Description of Artificial Sequence: Synthetic HDR
gagaacgctcgaccttcgatgtgtgtgt

SEQ ID NO: 394 - DNA, 28 bp, Artificial Sequence
  Description of Artificial Sequence: Synthetic HDR
gagaacgctccagcactgatgtgtgtgt

SEQ ID NO: 395 - DNA, 28 bp, Artificial Sequence
  Description of Artificial Sequence: Synthetic HDR
tccatgtcggtcctgatgctgtgtgtgt

SEQ ID NO: 396 - DNA, 28 bp, Artificial Sequence
  Description of Artificial Sequence: Synthetic HDR
tccatgtcggtcctgctgatgtgtgtgt

SEQ ID NO: 397 - DNA, 29 bp, Artificial Sequence
  Description of Artificial Sequence: Synthetic HDR
atggactctccagcgttctctgtgtgtgt

SEQ ID NO: 398 - DNA, 28 bp, Artificial Sequence
  Description of Artificial Sequence: Synthetic HDR
atggaaggtccaacgttctcgtgtgtgt

SEQ ID NO: 399 - DNA, 28 bp, Artificial Sequence
  Description of Artificial Sequence: Synthetic HDR
tccatggcggtcctgatgctgtgtgtgt

SEQ ID NO: 400 - DNA, 28 bp, Artificial Sequence
  Description of Artificial Sequence: Synthetic HDR
tccatgacggtcctgatgctgtgtgtgt

SEQ ID NO: 401 - DNA, 28 bp, Artificial Sequence
  Description of Artificial Sequence: Synthetic HDR
tccatgtcgatcctgatgctgtgtgtgt

SEQ ID NO: 402 - DNA, 28 bp, Artificial Sequence
  Description of Artificial Sequence: Synthetic HDR
tccatgtcgctcctgatgctgtgtgtgt

SEQ ID NO: 403 - DNA, 28 bp, Artificial Sequence
  Description of Artificial Sequence: Synthetic HDR
tccatgtcgttcctgatgctgtgtgtgt

SEQ ID NO: 404 - DNA, 15 bp, Artificial Sequence
  Description of Artificial Sequence: Synthetic HDR
gctagacgttagcgt

SEQ ID NO: 405 - DNA, 8 bp, Artificial Sequence
  Description of Artificial Sequence: Synthetic HDR
tcaacgtt

SEQ ID NO: 406 - DNA, 20 bp, Artificial Sequence
  Description of Artificial Sequence: Synthetic HDR
tccatgacgttcctgatgct

SEQ ID NO: 407 - DNA, 20 bp, Artificial Sequence
  Description of Artificial Sequence: Synthetic HDR
atcgactctcgagcgttctc

SEQ ID NO: 408 - DNA, 15 bp, Artificial Sequence
  Description of Artificial Sequence: Synthetic HDR
gcatgacgttgagct

SEQ ID NO: 409 - DNA, 8 bp, Artificial Sequence
  Description of Artificial Sequence: Synthetic HDR
tcagcgct

SEQ ID NO: 410 - DNA, 20 bp, Artificial Sequence
  Description of Artificial Sequence: Synthetic HDR
gagaacgctggaccttccat

SEQ ID NO: 411 - DNA, 20 bp, Artificial Sequence
  Description of Artificial Sequence: Synthetic HDR
gagaacgctcgaccttccat

SEQ ID NO: 412 - DNA, 20 bp, Artificial Sequence
  Description of Artificial Sequence: Synthetic HDR
gagaacgctcgaccttcgat

SEQ ID NO: 413 - DNA, 22 bp, Artificial Sequence
  Description of Artificial Sequence: Synthetic HDR
gagaacgctccagcactgatat

SEQ ID NO: 414 - DNA, 20 bp, Artificial Sequence
  Description of Artificial Sequence: Synthetic HDR
tccatgtcggtcctgatgct

SEQ ID NO: 415 - DNA, 20 bp, Artificial Sequence
  Description of Artificial Sequence: Synthetic HDR
tccatgtcggtcctgctgat

SEQ ID NO: 416 - DNA, 20 bp, Artificial Sequence
  Description of Artificial Sequence: Synthetic HDR
atggactctccagcgttctc

SEQ ID NO: 417 - DNA, 20 bp, Artificial Sequence
  Description of Artificial Sequence: Synthetic HDR
atggaaggtccaacgttctc

SEQ ID NO: 418 - DNA, 20 bp, Artificial Sequence
  Description of Artificial Sequence: Synthetic HDR
tccatggcggtcctgatgct

SEQ ID NO: 419 - DNA, 20 bp, Artificial Sequence
  Description of Artificial Sequence: Synthetic HDR
tccatgacggtcctgatgct

SEQ ID NO: 420 - DNA, 20 bp, Artificial Sequence
  Description of Artificial Sequence: Synthetic HDR
tccatgtcgatcctgatgct

SEQ ID NO: 421 - DNA, 20 bp, Artificial Sequence
  Description of Artificial Sequence: Synthetic HDR
tccatgtcgctcctgatgct

SEQ ID NO: 422 - DNA, 20 bp, Artificial Sequence
  Description of Artificial Sequence: Synthetic HDR
tccatgtcgttcctgatgct

SEQ ID NO: 423 - DNA, 15 bp, Artificial Sequence
  Description of Artificial Sequence: Synthetic HDR
gctagacgttagcgt

SEQ ID NO: 424 - DNA, 8 bp, Artificial Sequence
  Description of Artificial Sequence: Synthetic HDR
tcaacgtt

SEQ ID NO: 425 - DNA, 20 bp, Artificial Sequence
  Description of Artificial Sequence: Synthetic HDR
tccatgacgttcctgatgct

SEQ ID NO: 426 - DNA, 20 bp, Artificial Sequence
  Description of Artificial Sequence: Synthetic HDR
atcgactctcgagcgttctc

SEQ ID NO: 427 - DNA, 15 bp, Artificial Sequence
  Description of Artificial Sequence: Synthetic HDR
gcatgacgttgagct

SEQ ID NO: 428 - DNA, 8 bp, Artificial Sequence
  Description of Artificial Sequence: Synthetic HDR
gcagcgct

SEQ ID NO: 429 - DNA, 20 bp, Artificial Sequence
  Description of Artificial Sequence: Synthetic HDR
gagaacgctggaccttccat

SEQ ID NO: 430 - DNA, 20 bp, Artificial Sequence
  Description of Artificial Sequence: Synthetic HDR
gagaacgctcgaccttccat

SEQ ID NO: 431 - DNA, 20 bp, Artificial Sequence
  Description of Artificial Sequence: Synthetic HDR
gagaacgctcgaccttcgat

SEQ ID NO: 432 - DNA, 20 bp, Artificial Sequence
  Description of Artificial Sequence: Synthetic HDR
gagaacgctccagcactgat

SEQ ID NO: 433 - DNA, 20 bp, Artificial Sequence
  Description of Artificial Sequence: Synthetic HDR
tccatgtcggtcctgatgct

SEQ ID NO: 434 - DNA, 20 bp, Artificial Sequence
  Description of Artificial Sequence: Synthetic HDR
tccatgtcggtcctgctgat

SEQ ID NO: 435 - DNA, 20 bp, Artificial Sequence
  Description of Artificial Sequence: Synthetic HDR
atggactctccagcgttctc

SEQ ID NO: 436 - DNA, 20 bp, Artificial Sequence
  Description of Artificial Sequence: Synthetic HDR
atggaaggtccaacgttctc

SEQ ID NO: 437 - DNA, 20 bp, Artificial Sequence
  Description of Artificial Sequence: Synthetic HDR
tccatggcggtcctgatgct

SEQ ID NO: 438 - DNA, 20 bp, Artificial Sequence
  Description of Artificial Sequence: Synthetic HDR
tccatgacggtcctgatgct

SEQ ID NO: 439 - DNA, 20 bp, Artificial Sequence
  Description of Artificial Sequence: Synthetic HDR
tccatgtcgatcctgatgct

SEQ ID NO: 440 - DNA, 20 bp, Artificial Sequence
  Description of Artificial Sequence: Synthetic HDR
tccatgtcgctcctgatgct

SEQ ID NO: 441 - DNA, 20 bp, Artificial Sequence
  Description of Artificial Sequence: Synthetic HDR
tccatgtcgttcctgatgct

SEQ ID NO: 442 - DNA, 30 bp, Artificial Sequence
  Description of Artificial Sequence: Synthetic HDR
gctagacgttagcgtgctagacgttagcgt

SEQ ID NO: 443 - DNA, 28 bp, Artificial Sequence
  Description of Artificial Sequence: Synthetic HDR
tcaacgtttccatgacgttcctgatgct

SEQ ID NO: 444 - DNA, 40 bp, Artificial Sequence
  Description of Artificial Sequence: Synthetic HDR
tccatgacgttcctgatgcttccatgacgttcctgatgct

SEQ ID NO: 445 - DNA, 40 bp, Artificial Sequence
  Description of Artificial Sequence: Synthetic HDR
atcgactctcgagcgttctcatcgactctcgagcgttctc

SEQ ID NO: 446 - DNA, 30 bp, Artificial Sequence
  Description of Artificial Sequence: Synthetic HDR
gcatgacgttgagctgcatgacgttgagct

SEQ ID NO: 447 - DNA, 16 bp, Artificial Sequence
  Description of Artificial Sequence: Synthetic HDR
tcagcgcttcagcgct

SEQ ID NO: 448 - DNA, 40 bp, Artificial Sequence
  Description of Artificial Sequence: Synthetic HDR
gagaacgctggaccttccatgagaacgctcgaccttccat

SEQ ID NO: 449 - DNA, 40 bp, Artificial Sequence
  Description of Artificial Sequence: Synthetic HDR
gagaacgctcgaccttccatgagaacgctcgaccttcgat

SEQ ID NO: 450 - DNA, 40 bp, Artificial Sequence
  Description of Artificial Sequence: Synthetic HDR
gagaacgctcgaccttcgatgagaacgctccagcactgat

SEQ ID NO: 451 - DNA, 40 bp, Artificial Sequence
  Description of Artificial Sequence: Synthetic HDR
gagaacgctccagcactgattccatgtcggtcctgatgct

SEQ ID NO: 452 - DNA, 40 bp, Artificial Sequence
  Description of Artificial Sequence: Synthetic HDR
tccatgtcggtcctgatgcttccatgtcggtcctgctgat

SEQ ID NO: 453 - DNA, 40 bp, Artificial Sequence
  Description of Artificial Sequence: Synthetic HDR
tccatgtcggtcctgctgatatggactctccagcgttctc

SEQ ID NO: 454 - DNA, 40 bp, Artificial Sequence
  Description of Artificial Sequence: Synthetic HDR
atggactctccagcgttctcatggaaggtccaacgttctc

SEQ ID NO: 455 - DNA, 40 bp, Artificial Sequence
  Description of Artificial Sequence: Synthetic HDR
atggaaggtccaacgttctctccatggcggtcctgatgct

SEQ ID NO: 456 - DNA, 40 bp, Artificial Sequence
  Description of Artificial Sequence: Synthetic HDR
tccatggcggtcctgatgcttccatgacggtcctgatgct

SEQ ID NO: 457 - DNA, 40 bp, Artificial Sequence
  Description of Artificial Sequence: Synthetic HDR
tccatgacggtcctgatgcttccatgtcgatcctgatgct

SEQ ID NO: 458 - DNA, 40 bp, Artificial Sequence
  Description of Artificial Sequence: Synthetic HDR
tccatgtcgatcctgatgcttccatgtcgctcctgatgct

SEQ ID NO: 459 - DNA, 40 bp, Artificial Sequence
  Description of Artificial Sequence: Synthetic HDR
tccatgtcgctcctgatgcttccatgtcgttcctgatgct

SEQ ID NO: 460 - DNA, 40 bp, Artificial Sequence
  Description of Artificial Sequence: Synthetic HDR
tccatgtcgttcctgatgcttccatgacgtccctgatgct

SEQ ID NO: 461 - DNA, 31 bp, Artificial Sequence
  Description of Artificial Sequence: Synthetic HDR
gctagacgttagcgtttcgctaacgtctagc

SEQ ID NO: 462 - DNA, 16 bp, Artificial Sequence
  Description of Artificial Sequence: Synthetic HDR
tcaacgttaacgttga

SEQ ID NO: 463 - DNA, 32 bp, Artificial Sequence
  Description of Artificial Sequence: Synthetic HDR
ggtgcatcgatgcaggggggtcgagcgttctc

SEQ ID NO: 464 - DNA, 40 bp, Artificial Sequence
  Description of Artificial Sequence: Synthetic HDR
tccatgacgttcctgatgctagcatcaggaacgtcatgga

SEQ ID NO: 465 - DNA, 40 bp, Artificial Sequence
  Description of Artificial Sequence: Synthetic HDR
atcgactctcgagcgttctcgagaacgctcgagagtcgat

SEQ ID NO: 466 - DNA, 30 bp, Artificial Sequence
  Description of Artificial Sequence: Synthetic HDR
gcatgacgttgagctagctcaacgtcatgc

SEQ ID NO: 467 - DNA, 16 bp, Artificial Sequence
  Description of Artificial Sequence: Synthetic HDR
tcagcgctagcgctga

SEQ ID NO: 468 - DNA, 40 bp, Artificial Sequence
  Description of Artificial Sequence: Synthetic HDR
gagaacgctggaccttccatatggaaggtccagcgttctc

SEQ ID NO: 469 - DNA, 40 bp, Artificial Sequence
  Description of Artificial Sequence: Synthetic HDR
gagaacgctcgaccttccatatggaaggtcgagcgttctc

SEQ ID NO: 470 - DNA, 40 bp, Artificial Sequence
  Description of Artificial Sequence: Synthetic HDR
gagaacgctcgaccttcgatatcgaaggtcgagcgttcac

SEQ ID NO: 471 - DNA, 40 bp, Artificial Sequence
  Description of Artificial Sequence: Synthetic HDR
gagaacgctccagcactgatatcagtgctggagcgttcac

SEQ ID NO: 472 - DNA, 30 bp, Artificial Sequence
  Description of Artificial Sequence: Synthetic HDR
tccatgtcggtcctgatgctaggtgcagcc

SEQ ID NO: 473 - DNA, 26 bp, Artificial Sequence
  Description of Artificial Sequence: Synthetic HDR
tccatgtcggtcctgctgatcatgga

SEQ ID NO: 474 - DNA, 29 bp, Artificial Sequence
  Description of Artificial Sequence: Synthetic HDR
atggactctccagcgttctcagagtccta

SEQ ID NO: 475 - DNA, 33 bp, Artificial Sequence
  Description of Artificial Sequence: Synthetic HDR
atggaaggtccaacgttctcttggaccttccat

SEQ ID NO: 476 - DNA, 33 bp, Artificial Sequence
  Description of Artificial Sequence: Synthetic HDR
tccatggcggtcctgatgctaaaccgccatgga

SEQ ID NO: 477 - DNA, 33 bp, Artificial Sequence
  Description of Artificial Sequence: Synthetic HDR
tccatgacggtcctgatgcttcaggaccgacat

SEQ ID NO: 478 - DNA, 26 bp, Artificial Sequence
  Description of Artificial Sequence: Synthetic HDR
tccatgtcgatcctgatgctatcgac

SEQ ID NO: 479 - DNA, 26 bp, Artificial Sequence
  Description of Artificial Sequence: Synthetic HDR
tccatgtcgctcctgatgctcatgga

SEQ ID NO: 480 - DNA, 33 bp, Artificial Sequence
  Description of Artificial Sequence: Synthetic HDR
tccatgtcgttcctgatgctggaacgacatgga

SEQ ID NO: 481 - DNA, 18 bp, Artificial Sequence
  Description of Artificial Sequence: Synthetic HDR
ctcgagctcgagctcgag

SEQ ID NO: 482 - DNA, 18 bp, Artificial Sequence
  Description of Artificial Sequence: Synthetic HDR
atcgagatcgagatcgag

SEQ ID NO: 483 - DNA, 18 bp, Artificial Sequence
  Description of Artificial Sequence: Synthetic HDR
ctcgagctcgagctcgag

SEQ ID NO: 484 - DNA, 18 bp, Artificial Sequence
  Description of Artificial Sequence: Synthetic HDR
atcgatatcgatatcgat

SEQ ID NO: 485 - DNA, 18 bp, Artificial Sequence
  Description of Artificial Sequence: Synthetic HDR
ctcgatctcgatctcgat

SEQ ID NO: 486 - DNA, 12 bp, Artificial Sequence
  Description of Artificial Sequence: Synthetic HDR
atcgagctcgag

SEQ ID NO: 487 - DNA, 12 bp, Artificial Sequence
  Description of Artificial Sequence: Synthetic HDR
atcgagatcgag

SEQ ID NO: 488 - DNA, 12 bp, Artificial Sequence
  Description of Artificial Sequence: Synthetic HDR
atcgagctcgag

SEQ ID NO: 489 - DNA, 12 bp, Artificial Sequence
  Description of Artificial Sequence: Synthetic HDR
atcgatatcgat

SEQ ID NO: 490 - DNA, 12 bp, Artificial Sequence
  Description of Artificial Sequence: Synthetic HDR
ctcgatctcgat

SEQ ID NO: 491 - DNA, 18 bp, Artificial Sequence
  Description of Artificial Sequence: Synthetic HDR
atcgagctcgagatcgag

SEQ ID NO: 492 - DNA, 18 bp, Artificial Sequence
  Description of Artificial Sequence: Synthetic HDR
atcgagatcgagatcgag

SEQ ID NO: 493 - DNA, 18 bp, Artificial Sequence
  Description of Artificial Sequence: Synthetic HDR
atcgatctcgagatcgat

SEQ ID NO: 494 - DNA, 20 bp, Artificial Sequence
  Description of Artificial Sequence: Synthetic HDR
ggtgcatcgatgcagggggg

SEQ ID NO: 495 - DNA, 20 bp, Artificial Sequence
  Description of Artificial Sequence: Synthetic HDR
ggtgcagcggtgcagggggg

SEQ ID NO: 496 - DNA, 20 bp, Artificial Sequence
  Description of Artificial Sequence: Synthetic HDR
ggtgcaccggtgcagggggg

SEQ ID NO: 497 - DNA, 20 bp, Artificial Sequence
  Description of Artificial Sequence: Synthetic HDR
ggtgtgtcgatgcagggggg

SEQ ID NO: 498 - DNA, 20 bp, Artificial Sequence
  Description of Artificial Sequence: Synthetic HDR
ggtgcatcgacgcagggggg

SEQ ID NO: 499 - DNA, 20 bp, Artificial Sequence
  Description of Artificial Sequence: Synthetic HDR
ggtgcaccgatgcagggggg

SEQ ID NO: 500 - DNA, 22 bp, Artificial Sequence
  Description of Artificial Sequence: Synthetic HDR
ggggtgcatcgatgcagggggg

SEQ ID NO: 501 - DNA, 17 bp, Artificial Sequence
  Description of Artificial Sequence: Synthetic HDR
tgcatcgatgcaggggg

SEQ ID NO: 502 - DNA, 20 bp, Artificial Sequence
  Description of Artificial Sequence: Synthetic HDR
aatgcatcgatgcagggggg

SEQ ID NO: 503 - DNA, 18 bp, Artificial Sequence
  Description of Artificial Sequence: Synthetic HDR
tgcatcgatgcagggggg

SEQ ID NO: 504 - DNA, 20 bp, Artificial Sequence
  Description of Artificial Sequence: Synthetic HDR
ggtgcaccggtgcagggggg

SEQ ID NO: 505 - DNA, 20 bp, Artificial Sequence
  Description of Artificial Sequence: Synthetic HDR
ggtgcatcgatgcagggggg

SEQ ID NO: 506 - DNA, 20 bp, Artificial Sequence
  Description of Artificial Sequence: Synthetic HDR
ggtgcagcggtgcagggggg

SEQ ID NO: 507 - DNA, 20 bp, Artificial Sequence
  Description of Artificial Sequence: Synthetic HDR
ggtgcaccggtgcagggggg

SEQ ID NO: 508 - DNA, 20 bp, Artificial Sequence
  Description of Artificial Sequence: Synthetic HDR
ggtgtgtcgatgcagggggg

SEQ ID NO: 509 - DNA, 20 bp, Artificial Sequence
  Description of Artificial Sequence: Synthetic HDR
ggtgcatcgacgcagggggg

SEQ ID NO: 510 - DNA, 20 bp, Artificial Sequence
  Description of Artificial Sequence: Synthetic HDR
ggtgcaccgatgcagggggg

SEQ ID NO: 511 - DNA, 17 bp, Artificial Sequence
  Description of Artificial Sequence: Synthetic HDR
tgcatcgatgcaggggg

SEQ ID NO: 512 - DNA, 20 bp, Artificial Sequence
  Description of Artificial Sequence: Synthetic HDR
aatgcatcgatgcagggggg

SEQ ID NO: 513 - DNA, 18 bp, Artificial Sequence
  Description of Artificial Sequence: Synthetic HDR
tgcatcgatgcagggggg

SEQ ID NO: 514 - DNA, 20 bp, Artificial Sequence
  Description of Artificial Sequence: Synthetic HDR
ggtgcaccggtgcagggggg

SEQ ID NO: 515 - DNA, 28 bp, Artificial Sequence
  Description of Artificial Sequence: Synthetic HDR
ggtgcatcgatgcaggggggaaaaaaaa

SEQ ID NO: 516 - DNA, 28 bp, Artificial Sequence
  Description of Artificial Sequence: Synthetic HDR
ggtgcagcggtgcaggggggaaaaaaaa

SEQ ID NO: 517 - DNA, 28 bp, Artificial Sequence
  Description of Artificial Sequence: Synthetic HDR
ggtgcaccggtgcaggggggaaaaaaaa

SEQ ID NO: 518 - DNA, 28 bp, Artificial Sequence
  Description of Artificial Sequence: Synthetic HDR
ggtgtgtcgatgcaggggggaaaaaaaa

SEQ ID NO: 519 - DNA, 28 bp, Artificial Sequence
  Description of Artificial Sequence: Synthetic HDR
ggtgcatcgacgcaggggggaaaaaaaa

SEQ ID NO: 520 - DNA, 28 bp, Artificial Sequence
  Description of Artificial Sequence: Synthetic HDR
ggtgcaccgatgcaggggggaaaaaaaa

SEQ ID NO: 521 - DNA, 30 bp, Artificial Sequence
  Description of Artificial Sequence: Synthetic HDR
ggggtgcatcgatgcaggggggaaaaaaaa

SEQ ID NO: 522 - DNA, 25 bp, Artificial Sequence
  Description of Artificial Sequence: Synthetic HDR
tgcatcgatgcagggggaaaaaaaa

SEQ ID NO: 523 - DNA, 28 bp, Artificial Sequence
  Description of Artificial Sequence: Synthetic HDR
aatgcatcgatgcaggggggaaaaaaaa

SEQ ID NO: 524 - DNA, 26 bp, Artificial Sequence
  Description of Artificial Sequence: Synthetic HDR
tgcatcgatgcaggggggaaaaaaaa

SEQ ID NO: 525 - DNA, 28 bp, Artificial Sequence
  Description of Artificial Sequence: Synthetic HDR
ggtgcaccggtgcaggggggaaaaaaaa

SEQ ID NO: 526 - DNA, 30 bp, Artificial Sequence; identical to SEQ ID NO: 521
  Description of Artificial Sequence: Synthetic HDR

SEQ ID NO: 527 - DNA, 28 bp, Artificial Sequence
  Description of Artificial Sequence: Synthetic HDR
tcaacgttggtgcatcgatgcagggggg

SEQ ID NO: 528 - DNA, 28 bp, Artificial Sequence
  Description of Artificial Sequence: Synthetic HDR
tcaacgttggtgcagcggtgcagggggg

SEQ ID NO: 529 - DNA, 28 bp, Artificial Sequence
  Description of Artificial Sequence: Synthetic HDR
tcaacgttggtgcaccggtgcagggggg

SEQ ID NO: 530 - DNA, 28 bp, Artificial Sequence
  Description of Artificial Sequence: Synthetic HDR
tcaacgttggtgtgtcgatgcagggggg

SEQ ID NO: 531 - DNA, 28 bp, Artificial Sequence
  Description of Artificial Sequence: Synthetic HDR
tcaacgttggtgcatcgacgcagggggg

SEQ ID NO: 532 - DNA, 28 bp, Artificial Sequence
  Description of Artificial Sequence: Synthetic HDR
tcaacgttggtgcaccgatgcagggggg

SEQ ID NO: 533 - DNA, 29 bp, Artificial Sequence
  Description of Artificial Sequence: Synthetic HDR
tcaacgttgggtgcatcgatgcagggggg

SEQ ID NO: 534 - DNA, 25 bp, Artificial Sequence
  Description of Artificial Sequence: Synthetic HDR
tcaacgtttgcatcgatgcaggggg

SEQ ID NO: 535 - DNA, 28 bp, Artificial Sequence
  Description of Artificial Sequence: Synthetic HDR
tcaacgttaatgcatcgatgcagggggg

SEQ ID NO: 536 - DNA, 26 bp, Artificial Sequence
  Description of Artificial Sequence: Synthetic HDR
tcaacgtttgcatcgatgcagggggg

SEQ ID NO: 537 - DNA, 28 bp, Artificial Sequence
  Description of Artificial Sequence: Synthetic HDR
tcaacgttggtgcaccggtgcagggggg

SEQ ID NO: 538 - DNA, 20 bp, Artificial Sequence
  Description of Artificial Sequence: Synthetic HDR
ggtgcatcgatgcagggggg

SEQ ID NO: 539 - DNA, 21 bp, Artificial Sequence
  Description of Artificial Sequence: Synthetic HDR
ggtgcagcggtcgcagggggg

SEQ ID NO: 540 - DNA, 20 bp, Artificial Sequence
  Description of Artificial Sequence: Synthetic HDR
ggtgcaccggtgcagggggg

SEQ ID NO: 541 - DNA, 20 bp, Artificial Sequence
  Description of Artificial Sequence: Synthetic HDR
ggtgtgtcgatgcagggggg

SEQ ID NO: 542 - DNA, 20 bp, Artificial Sequence
  Description of Artificial Sequence: Synthetic HDR
ggtgcatcgacgcagggggg

SEQ ID NO: 543 - DNA, 20 bp, Artificial Sequence
  Description of Artificial Sequence: Synthetic HDR
ggtgcaccgatgcagggggg

SEQ ID NO: 544 - DNA, 22 bp, Artificial Sequence
  Description of Artificial Sequence: Synthetic HDR
ggggtgcatcgatgcagggggg

SEQ ID NO: 545 - DNA, 17 bp, Artificial Sequence
  Description of Artificial Sequence: Synthetic HDR
tgcatcgatgcaggggg

SEQ ID NO: 546 - DNA, 20 bp, Artificial Sequence
  Description of Artificial Sequence: Synthetic HDR
aatgcatcgatgcagggggg

SEQ ID NO: 547 - DNA, 18 bp, Artificial Sequence
  Description of Artificial Sequence: Synthetic HDR
tgcatcgaatcagggggg

SEQ ID NO: 548 - DNA, 40 bp, Artificial Sequence
  Description of Artificial Sequence: Synthetic HDR
tatatatccccccggtgcaccggtgcaggggggatatata

SEQ ID NO: 549 - DNA, 17 bp, Artificial Sequence
  Description of Artificial Sequence: Synthetic HDR
tgcatcgatgcaggggg

SEQ ID NO: 550 - DNA, 20 bp, Artificial Sequence
  Description of Artificial Sequence: Synthetic HDR
aatgcatcgatgcagggggg

SEQ ID NO: 551 - DNA, 18 bp, Artificial Sequence
  Description of Artificial Sequence: Synthetic HDR
tgcatcgatgcagggggg

SEQ ID NO: 552 - DNA, 20 bp, Artificial Sequence
  Description of Artificial Sequence: Synthetic HDR
atcgactctcgagcgttctc

SEQ ID NO: 553 - DNA, 12 bp, Artificial Sequence
  Description of Artificial Sequence: Synthetic HDR
tcgagcgttctc

SEQ ID NO: 554 - DNA, 19 bp, Artificial Sequence
  Description of Artificial Sequence: Synthetic HDR
tcgactctcgagcgttctc

SEQ ID NO: 555 - DNA, 16 bp, Artificial Sequence
  Description of Artificial Sequence: Synthetic HDR
actctcgagcgttctc

SEQ ID NO: 556 - DNA, 14 bp, Artificial Sequence
  Description of Artificial Sequence: Synthetic HDR
tctcgagcgttctc

SEQ ID NO: 557 - DNA, 12 bp, Artificial Sequence
  Description of Artificial Sequence: Synthetic HDR
ctcgagcgttct

SEQ ID NO: 558 - DNA, 12 bp, Artificial Sequence
  Description of Artificial Sequence: Synthetic HDR
tcgaggcttctc

SEQ ID NO: 559 - DNA, 12 bp, Artificial Sequence
  Description of Artificial Sequence: Synthetic HDR
gcgaggcttctc

SEQ ID NO: 560 - DNA, 12 bp, Artificial Sequence
  Description of Artificial Sequence: Synthetic HDR
tcgatgcttctc

SEQ ID NO: 561 - DNA, 12 bp, Artificial Sequence
  Description of Artificial Sequence: Synthetic HDR
tgcttcgagctc

SEQ ID NO: 562 - DNA, 12 bp, Artificial Sequence
  Description of Artificial Sequence: Synthetic HDR
tcgtttgttctc

SEQ ID NO: 563 - DNA, 12 bp, Artificial Sequence
  Description of Artificial Sequence: Synthetic HDR
tcgtatgtactc

SEQ ID NO: 564 - DNA, 12 bp, Artificial Sequence
  Description of Artificial Sequence: Synthetic HDR
ttgttcgttctc

SEQ ID NO: 565 - DNA, 12 bp, Artificial Sequence
  Description of Artificial Sequence: Synthetic HDR
ttgttcgtactc

SEQ ID NO: 566 - DNA, 20 bp, Artificial Sequence
  Description of Artificial Sequence: Synthetic HDR
atcgactctcgagcgttctc

SEQ ID NO: 567 - DNA, 12 bp, Artificial Sequence
  Description of Artificial Sequence: Synthetic HDR
tcgagcgttctc

SEQ ID NO: 568 - DNA, 19 bp, Artificial Sequence
  Description of Artificial Sequence: Synthetic HDR
tcgactctcgagcgttctc

SEQ ID NO: 569 - DNA, 16 bp, Artificial Sequence
  Description of Artificial Sequence: Synthetic HDR
actctcgagcgttctc

SEQ ID NO: 570 - DNA, 14 bp, Artificial Sequence
  Description of Artificial Sequence: Synthetic HDR
tctcgagcgttctc

SEQ ID NO: 571 - DNA, 12 bp, Artificial Sequence
  Description of Artificial Sequence: Synthetic HDR
ctcgagcgttct

SEQ ID NO: 572 - DNA, 12 bp, Artificial Sequence
  Description of Artificial Sequence: Synthetic HDR
tcgaggcttctc

SEQ ID NO: 573 - DNA, 12 bp, Artificial Sequence
  Description of Artificial Sequence: Synthetic HDR
gcgaggcttctc

SEQ ID NO: 574 - DNA, 12 bp, Artificial Sequence
  Description of Artificial Sequence: Synthetic HDR
tcgatgcttctc

SEQ ID NO: 575 - DNA, 12 bp, Artificial Sequence
  Description of Artificial Sequence: Synthetic HDR
tgcttcgagctc

SEQ ID NO: 576 - DNA, 12 bp, Artificial Sequence
  Description of Artificial Sequence: Synthetic HDR
tcgtttgttctc

SEQ ID NO: 577 - DNA, 12 bp, Artificial Sequence
  Description of Artificial Sequence: Synthetic HDR
tcgtatgtactc

SEQ ID NO: 578 - DNA, 12 bp, Artificial Sequence
  Description of Artificial Sequence: Synthetic HDR
ttgttcgttctc

SEQ ID NO: 579 - DNA, 12 bp, Artificial Sequence
  Description of Artificial Sequence: Synthetic HDR
ttgttcgtactc

SEQ ID NO: 580 - DNA, 40 bp, Artificial Sequence; identical to SEQ ID NO: 445
  Description of Artificial Sequence: Synthetic HDR

SEQ ID NO: 581 - DNA, 22 bp, Artificial Sequence
  Description of Artificial Sequence: Synthetic HDR
aaccaaccaatcgagcgttctc

SEQ ID NO: 582 - DNA, 26 bp, Artificial Sequence
  Description of Artificial Sequence: Synthetic HDR
aaccaaccaaactctcgagcgttctc

SEQ ID NO: 583 - DNA, 24 bp, Artificial Sequence
  Description of Artificial Sequence: Synthetic HDR
aaccaaccaatctcgagcgttctc

SEQ ID NO: 584 - DNA, 22 bp, Artificial Sequence
  Description of Artificial Sequence: Synthetic HDR
aaccaaccaactcgagcgttct

SEQ ID NO: 585 - DNA, 22 bp, Artificial Sequence
  Description of Artificial Sequence: Synthetic HDR
aaccaaccaatcgaggcttctc

SEQ ID NO: 586 - DNA, 22 bp, Artificial Sequence
  Description of Artificial Sequence: Synthetic HDR
aaccaaccaagcgaggcttctc

SEQ ID NO: 587 - DNA, 22 bp, Artificial Sequence
  Description of Artificial Sequence: Synthetic HDR
aaccaaccaatcgatgcttctc

SEQ ID NO: 588 - DNA, 22 bp, Artificial Sequence
  Description of Artificial Sequence: Synthetic HDR
aaccaaccaatgcttcgagctc

SEQ ID NO: 589 - DNA, 22 bp, Artificial Sequence
  Description of Artificial Sequence: Synthetic HDR
aaccaaccaatcgtttgttctc

SEQ ID NO: 590 - DNA, 22 bp, Artificial Sequence
  Description of Artificial Sequence: Synthetic HDR
aaccaaccaatcgtatgtactc

SEQ ID NO: 591 - DNA, 22 bp, Artificial Sequence
  Description of Artificial Sequence: Synthetic HDR
aaccaaccaattgttcgttctc

SEQ ID NO: 592 - DNA, 22 bp, Artificial Sequence
  Description of Artificial Sequence: Synthetic HDR
aaccaaccaattgttcgtactc

SEQ ID NO: 593 - DNA, 21 bp, Artificial Sequence
  Description of Artificial Sequence: Synthetic HDR
aatcgactctcgagcgttctc

SEQ ID NO: 594 - DNA, 12 bp, Artificial Sequence
  Description of Artificial Sequence: Synthetic HDR
tcgagcgttctc

SEQ ID NO: 595 - DNA, 19 bp, Artificial Sequence
  Description of Artificial Sequence: Synthetic HDR
tcgactctcgagcgttctc

SEQ ID NO: 596 - DNA, 16 bp, Artificial Sequence
  Description of Artificial Sequence: Synthetic HDR
actctcgagcgttctc

SEQ ID NO: 597 - DNA, 14 bp, Artificial Sequence
  Description of Artificial Sequence: Synthetic HDR
tctcgagcgttctc

SEQ ID NO: 598 - DNA, 12 bp, Artificial Sequence
  Description of Artificial Sequence: Synthetic HDR
ctcgagcgttct

SEQ ID NO: 599 - DNA, 12 bp, Artificial Sequence
  Description of Artificial Sequence: Synthetic HDR
tcgaggcttctc

SEQ ID NO: 600 - DNA, 12 bp, Artificial Sequence
  Description of Artificial Sequence: Synthetic HDR
tcgatgcttctc

SEQ ID NO: 601 - DNA, 12 bp, Artificial Sequence
  Description of Artificial Sequence: Synthetic HDR
tgcttcgagctc

SEQ ID NO: 602 - DNA, 12 bp, Artificial Sequence
  Description of Artificial Sequence: Synthetic HDR
tcgtttgttctc

SEQ ID NO: 603 - DNA, 12 bp, Artificial Sequence
  Description of Artificial Sequence: Synthetic HDR
tcgtatgtactc

SEQ ID NO: 604 - DNA, 12 bp, Artificial Sequence
  Description of Artificial Sequence: Synthetic HDR
ttgttcgttctc

SEQ ID NO: 605 - DNA, 12 bp, Artificial Sequence
  Description of Artificial Sequence: Synthetic HDR
ttgttcgtactc

SEQ ID NO: 606 - DNA, 30 bp, Artificial Sequence
  Description of Artificial Sequence: Synthetic HDR
atcgactctcgagcgttctctttttttttt

SEQ ID NO: 607 - DNA, 22 bp, Artificial Sequence
  Description of Artificial Sequence: Synthetic HDR
tcgagcgttctctttttttttt

SEQ ID NO: 608 - DNA, 29 bp, Artificial Sequence
  Description of Artificial Sequence: Synthetic HDR
tcgactctcgagcgttctctttttttttt

SEQ ID NO: 609 - DNA, 26 bp, Artificial Sequence
  Description of Artificial Sequence: Synthetic HDR
actctcgagcgttctctttttttttt

SEQ ID NO: 610 - DNA, 24 bp, Artificial Sequence
  Description of Artificial Sequence: Synthetic HDR
tctcgagcgttctctttttttttt

SEQ ID NO: 611 - DNA, 22 bp, Artificial Sequence
  Description of Artificial Sequence: Synthetic HDR
ctcgagcgttcttttttttttt

SEQ ID NO: 612 - DNA, 22 bp, Artificial Sequence
  Description of Artificial Sequence: Synthetic HDR
tcgaggcttctctttttttttt

SEQ ID NO: 613 - DNA, 22 bp, Artificial Sequence
  Description of Artificial Sequence: Synthetic HDR
gcgaggcttctctttttttttt

SEQ ID NO: 614 - DNA, 22 bp, Artificial Sequence
  Description of Artificial Sequence: Synthetic HDR
tcgatgcttctctttttttttt

SEQ ID NO: 615 - DNA, 21 bp, Artificial Sequence
  Description of Artificial Sequence: Synthetic HDR
tgcttcgagctcttttttttt

SEQ ID NO: 616 - DNA, 22 bp, Artificial Sequence
  Description of Artificial Sequence: Synthetic HDR
tcgtttgttctctttttttttt

SEQ ID NO: 617 - DNA, 22 bp, Artificial Sequence
  Description of Artificial Sequence: Synthetic HDR
tcgtatgtactctttttttttt

SEQ ID NO: 618 - DNA, 22 bp, Artificial Sequence
  Description of Artificial Sequence: Synthetic HDR
ttgttcgttctctttttttttt

SEQ ID NO: 619 - DNA, 22 bp, Artificial Sequence
  Description of Artificial Sequence: Synthetic HDR
ttgttcgtactctttttttttt

SEQ ID NO: 620 - DNA, 21 bp, Artificial Sequence
  Description of Artificial Sequence: Synthetic HDR
gctagacgttagcgtaacgtt

(19) United States
(12) Patent Application Publication
(10) Pub. No.: US 2004/0052763 A1
(43) Pub. Date: Mar. 18, 2004

(54) Immunostimulatory RNA/DNA hybrid molecules

(76) Inventors: James J. Mond, Silver Spring, MD (US); Michael Flora, Mt. Airy, MD (US); Dennis M. Klinman, Potomac, MD (US)

Correspondence Address: FINNEGAN, HENDERSON, FARABOW, GARRETT & DUNNER, LLP, 1300 I STREET, NW, WASHINGTON, DC, 20005, US

(21) Appl. No.: 09/874,991

(22) Filed: Jun. 7, 2001

Related U.S. Application Data
(60) Provisional application No. 60/209,797, filed on Jun. 7, 2000.

(51) Int. Cl. A61K 48/00

(52) U.S. Cl. 424/93.2; 514/44

(57) ABSTRACT

The present invention provides immunological compositions and methods relating to immunostimulatory intra-strand DNA/RNA hybrid oligonucleotides (HDRs), optimally encoding one or more CpG motif, which may be an unmethylated CpG motif. Administration of these compounds, alone or in the context of one or more target antigens, promotes innate and antigen specific immunities.

### CROSS REFERENCE TO RELATED APPLICATIONS

This present application is based upon U.S. provisional application Serial No. 60/209,797, filed Jun. 7, 2000, priority to which is claimed under 35 U.S.C. § 119(e), the entire disclosure of which is incorporated by reference.

### FIELD OF THE INVENTION

The present invention relates to immunostimulatory RNA/DNA hybrid oligonucleotides and their use in enhancing an immune response, or inducing cytokines. The present invention further relates to a novel adjuvanting system comprising DNA, RNA, and/or RNA/DNA hybrid oligonucleotides containing CpG dinucleotides, which may be unmethylated CpG dinucleotides, conjugated to a high molecular weight polysaccharide or other polyvalent carrier.

### BACKGROUND

The use of nucleic acids as immunostimulatory molecules has recently gained acceptance. The immunoreactive properties of nucleic acids are determined by their base composition, modifications, and helical orientation. For example, humoral immune responses to cellular DNAs have been implicated in unusual DNA structures, such as Z-DNA, which can induce significant antibody responses in experimental animals. Double stranded nucleic acids comprising DNA, RNA, and inter-strand DNA:RNA hybrids all have the potential for generating a humoral immune response. Eliat and Anderson, Mol. Immunol. 31:1377 (1994). Indeed, antibodies directed against cellular DNA have long been implicated in the autoimmune condition systemic lupus erythematosus.

It is also known that DNA sequences containing certain unmethylated CpG sequences, sometimes called “CpG ODNs” (CpG oligodeoxynucleotides), are highly stimulatory of cells in the immune system, and can induce vigorous proliferation and immunoglobulin (Ig) production by B cells. See generally Klinman et al., Vaccine 17:19 (1999); and McCluskie and Davis, J. Immun. 161:4463 (1998) (each of which is incorporated herein by reference in its entirety). Interestingly, these unmethylated CpG dinucleotides are far more frequent in the genomes of bacteria and viruses than vertebrates and may contribute to vertebrates' innate immune responses to bacteria and viruses. Klinman et al., Proc. Natl. Acad. Sci. USA 93:2879 (1996); Yi et al. J. Immun. 157: 5394 (1996); Hua Liang et al., J. Clin. Invest. 98:1119 (1996); Krieg et al., Nature 374: 546 (1995), each of which is incorporated herein by reference in its entirety.

Since the interest in CpG DNA began, studies have focused on the possible mechanism of action. In mice, CpG DNA induces proliferation in almost all (>95%) B cells. These oligonucleotides stimulate immunoglobin (Ig) secretion and may act by increasing the secretion of IL-6from B cells. This B cell activation by CpG DNA is T cell independent and antigen non-specific. In addition to its direct effects on B cells, CpG DNA also directly activates monocytes, macrophages, and dendritic cells to secrete a variety of cytokines, including IL-6, IL-12, GMC-CSF, TNF-α, CSF, and interferons. These cytokines stimulate natural killer (NK) cells to secrete γ-interferon (IFN-γ) and have increased lytic activity. Examples of applications covering these aspects can be found in International Patent Applications WO 95/26204, WO 96/02555, WO 98/11211, WO 98/18810, WO 98/37919, WO 98/40100, WO 98/52581, and PCT/US98/047703; and U.S. Pat. No. 5,663,153, each of which is incorporated herein by reference in its entirety.

In light of the above observations, oligonucleotides, particularly those containing various formulations of CpG motifs, have frequently been suggested as vaccine adjuvants, or stimulants of global immune responses. Reviewed in Immunobiology of Bacterial CpG-DNA (Springer, 2000, H. Wagner ed.) (each of which is incorporated herein by reference in its entirety.) In practice, such oligonucleotides are somewhat effective but have been constructed entirely of DNA or DNA analogs. See, for example, Kreig et al., in Immunobiology of Bacterial CpG-DNA, cited above.

In addition to CpG-containing DNAs, a number of other polynucleotides have been evaluated as biological response modifiers. Perhaps the best example is poly (I,C) which is a potent inducer of interferon (IFN) production as well as a macrophage activator and inducer of NK activity. Its potent in vitro antitumor activity led to several clinical trials using poly (I,C) complexed with poly-L-lysine and carboxymethylcellulose (to reduce degradation by RNAse). (Talmadge, et al., Cancer res. 45:1058 (1985); Wiltrout, et a., J. Biol. Resp. Mod. 4:512 (1985) Krown, Sem. Oncol. 13:207 (1986); and Ewel, et al., Canc. Res. 52:3005 (1992)). In contrast to the CpG-based oligonucleotides, the immunostimulatory effects of poly (I,C) appear to be specific for the ribose sugar-based forms of these bases, since deoxyribose-based ply (I,C) was ineffective. Nevertheless, toxic side effects have thus far prevented poly (I,C) from becoming a useful therapeutic agent. In contrast, CpG based compositions may provide useful anti-cancer therapies, adjuvants, and modifiers of cytokine secretion profiles.

Thus, there exists a need for immunostimulatory oligonucleotides that optimally induce both global and specific immune responses, and that might be directed in their ability to induce T-cell dependent or B-cell dependent responses and/or specifically Th1 or Th2 responses. In addition, there is a need for methods utilizing these oligonucleotides as vaccine adjuvants and in the treatment of disease.

### SUMMARY OF THE INVENTION

The inventors have discovered that oligonucleotides comprising intra-strand hybrids of RNA and DNA, optionally encoding one or more CpG motifs, address these needs by providing highly efficacious global and antigen-specific immune stimulation. These and other advantages of the present invention, as well as additional inventive features, will be apparent from the description of the invention provided herein.

### DETAILED DESCRIPTION OF THE INVENTION

As discussed above, oligonucleotide sequences based solely on DNA and DNA derivatives display immunostimulatory activities. In contrast, the immunogenic and immunotherapeutic compositions and methods of the present invention relate to novel hybrid DNA/RNA oligonucleotides (HDRs). Surprisingly, these hybrid oligonucleotide sequences display different, and in some aspects, superior, immunostimulory characteristics than those based solely on DNA. This is particularly surprising in view of the inoperability of RNA-based molecules. Indeed, there is not a single report of a successful immune modulator based on RNA.

The mixed-backbone of ribose and deoxyribose nucleotides in the instant HDRs provides an efficacious alternative to the known immunostimulatory oligonucleotide compositions. Moreover, when compared to standard CpG polynucleotide formulations, the HDRs of the invention demonstrate increased activities in a variety of T cell-dependent applications, elicit more defined cytokine production profiles from B cells and other cell types, and are effective stimulants of T cell-independent immunity.

Without limitation to any particular theory of the invention, it is presently believed that the HDRs of the invention directly or indirectly influence cells of the immune system by altering the quantity or amount of stimulatory and inhibitory cytokines produced by cells of the immune system. These HDR-sensitive cells include macrophages, T cells, NK cells, and dendritic cells involved in both acquired and innate immunities (discussed at length in Ivan Roit, Essential Immunology (8^{th} Ed. 1994) (incorporated herein by reference in its entirety). In addition, for the purpose of this invention, global immunity refers to the overall sensitivity of a patient's immune response and its ability to mount effective defenses against any foreign entity, including inappropriately presented endogenous antigens.

Acquired immunity comprises a host's response to antigenic challenge by both foreign (e.g. allergens, pathogens, transplanted tissues) and self-derived (e.g. tumor antigens, autoantigens) antigens, and is preferably associated with a memory response. Acquired immunity encompasses both cell-mediated (e.g. cytotoxic activity) and humoral immunity (resulting in the production of antibodies) and generally depends on regulation by T cells and NK cells.

T cells play a central role in many aspects of acquired immunity, carrying out a variety of regulatory and defensive functions. When some T cells encounter an infected or cancerous cell, they recognize it as foreign and respond by acting as killer cells, killing the host's own cells as part of the cell-mediated immune response. Other T cells, designated helper T cells, respond to perceived foreign antigens by stimulating B cells to produce antibodies, or by suppressing certain aspects of a humoral or cellular immune response.

T helper cells (Th) orchestrate much of the immune response via the production of cytokines. Although generally identifiable as bearing the CD4 cell surface marker, these cells are functionally divided into Th1 or Th2 subpopulations according to the profile of cytokines they produce and their effect on other cells of the immune system.

The Th1 cells detect invading pathogens or cancerous host cells through a recognition system referred to as the T cell antigen receptor. Termed cellular immunity, Th1-related processes generally involve the activation of non-B cells and are frequently characterized by the production of IFN-γ. Nevertheless, although the Th1 system is primarily independent from the production of humoral antibodies, Th1 cytokines do promote immunoglobulin class switching to the IgG₂ₐ isotype.

Upon detection of a foreign antigen, most mature Th1 cells direct the release of IL-2, IL-3, IFN-γ, TNF-β, GM-CSF, high levels of TNF-α, MIP-1α, MIP-1β, and RANTES. These cytokines promote delayed-type hypersensitivity and general cell-mediated immunity. IL-2, for instance, is a T cell growth factor that promotes the production of a clone of additional T cells sensitive to the particular antigen that was initially detected. The sensitized T cells attach to and attack cells or pathogens containing the antigen.

In contrast, mature Th2 cells tend to promote the secretion of IL-3, IL4, IL-5, IL-6, IL-9, IL-10, IL-13, GM-CSF, and low levels of TNF-α. In addition, the Th2 response promotes humoral immunity by activating B cells, stimulating antibody production and secretion, and inducing class switching to IgA, IgG, and IgE isotypes.

In nature, the stimulation of B cells leading to a humoral or systemic immune response depends on the ability of the B cells to recognize specific antigens. B cells recognize antigens via specific receptors on their cell surface called immunoglobulins or antibodies. When an antigen attaches to the receptor site of a B cell, the B cell is stimulated to divide to form daughter cells. In the case of a T-cell independent antigen, such as a bacterial polysaccharide, the B cell activation results in a low level response, characterized by little, if any class switching or memory response. In contrast, T-cell dependent antigens stimulate receptors on both B cells and Th2 cells, resulting in a vigorous and complex humoral immune response. Specifically, cytokines such as IL-6, produced by stimulated Th2 cells, cause the B cells to mature and produce antibodies. Maturation includes class-switching from the primitive IgM isotype, the production of memory cells, and the selection of high affinity antigen binding specificities.

The Th1 and Th2-type cytokines also affect the Th populations themselves. For example, IL-12 and IFN-γ up regulate Th1 responses but down regulate Th2 cells. IL-12 itself promotes IFN-γ production, providing a positive feed back for IL-12 production by Th1 cells. In addition, NK cells also regulate Th1 and Th2 immunity by secreting IFN-γ. The signal for NK cells to secrete IFN-γ may be precipitated by cytokines released from antigen presenting cells in response to antigen but may also be directly or indirectly precipitated by the addition of the HDRs of the invention.

Nevertheless, irrespective of the mechanism, the HDRs of the invention can stimulate the production of cytokines characteristic of Th1 regulation, Th2 T regulation, or both—indicative of their efficacy in stimulating both humoral and cellular immunity.

In addition, induction of one type of immune response may allow for immune regulation because up regulation of one type of immune response may down regulate the other type of immune response. This immune regulation allows for customizing or tailoring of the type of immune response when administering the immunogenic compositions of the invention.

Moreover, given the wealth of knowledge in the art regarding the use of cytokines to favor (or reduce) particular facets of an immune response, the HDRs of the invention may be administered in conjunction with one or more cytokines. Thus, one or more cytokines or active portions of cytokines may be administered directly, as soluble factors, conjugates, or fusion proteins with antigen or other cytokines, or indirectly, as nucleic acids encoding one or more cytokine activities, to a patient in need of immune stimulation. For example, the compositions and methods disclosed in U.S. Pat. No. 5,874,085 to Mond and Snapper (incorporated herein by reference in its entirety) may be administered with the HDRs of the invention not only to promote a Th2 response, but also to direct isotype switching to predominantly IgA antibodies.

Similarly, the humoral arm of an HDR-mediated response may comprise a primarily IgG, response if the HDR is administered in conjunction with antigen, GM-CSF and IL-2, as taught in copending U.S. application Ser. No. 08/568,343 (incorporated herein by reference in its entirety). Moreover, the HDRs of the invention generally promote class switching to isotypes other than the IgE isotype. Consequently, the administration of an HDR with an allergen may ameliorate or prevent an allergic response. The allergen may be administered in association with an HDR of the invention or may be present in the environment of the organism to which an HDR is being administered.

In addition to the above methods for shaping and enhancing acquired immunities, the HDRs of the invention may also promote an increase in the effectiveness of innate immunity. As used herein, innate immunity is any effect on the immune system which is not intrinsically dependent on prior contact with antigen. Most broadly, this encompasses priming the acquired immunity system in the absence of antigen, for example, by increasing the number of naive or quiescent B, T, NK, or antigen presenting cells or, by increasing their sensitivity to subsequent stimulation.

Innate immunity further comprises that arm of the immune system which is not directly dependent on T or B lymphocytes. Macrophages, neutrophils and monocytes are important effector cells for innate immunity. Macrophages, for example, play an important role in the destruction of solid tumors, in part, through the production of reactive oxygen intermediates and the cytokine TNF. The macrophage's ability to destroy cells bearing foreign antigens is enhanced by other cytokines that attract or stimulate this cell type. NK cells, for example, may provide an important link between the acquired and innate responses by providing cytokines which attract or stimulate macrophages to destroy cells bearing foreign antigens. By analogy to the effects of CpG-containing ODNs, HDRs may increase the sensitivity of NK cells to IL-12, resulting in an increased release of cytokines such as IFN-γ from the NK population. Alternatively, or in addition, HDRs may initially act on antigen presenting cells (primarily macrophages and dendritic cells), which release cytokines that act on the NK cells. Nevertheless, irrespective of the underlying mechanisms, the administration of the HDRs of the invention to a host can promote innate immunity defenses against both pathogenic invasion and cancerous cells.

Hybrid DNA/RNA Oligonucleotides

The present invention provides synthetic HDR molecules of at least about 9 nucleotides in length, but which may be about 10 to 20, 20 to 50, 50 to 100 or more nucleotides in length, including any value subsumed within those ranges. For facilitating uptake into cells, less than 40 nucleotides may be advantageous. Each of the immunostimulatory polynucleotides comprises both RNA and DNA bases, which may include modified polynucleotides and nucleotide analogs. The HDRs may be single-stranded, but also encompass double-stranded, partially double-stranded, and self-complementary hair-pin structures.

In one embodiment, the HDR comprises a 5′ DNA portion and a 3′ RNA portion; in another embodiment the position of the two portions is reversed. A single HDR may contain multiple DNA and/or RNA portions. In one embodiment, a DNA portion is flanked by RNA portions. Each DNA portion comprises at least 1 nucleotide, but may comprise about 2 to 5, 5 to 10, 10 to 20, 20 to 50 or more nucleotides having a deoxyribose-phosphate backbone, or modification thereof, including any value subsumed within the recited ranges.

Each RNA portion of the HDR comprises at least 1 nucleotide, but may comprise about 2 to 6, 6 to 10, 10 to 20, 20 to 50 or more nucleotides having a ribose-phosphate backbone, or modification thereof, including any value subsumed within the recited ranges. The RNA portion may be of any base sequence (including a base sequence comprising all or part of a CpG sequence), for example, a run of purine bases. The bases may be of essentially uniform composition, e.g., polyadenine (poly A), polyuracil (poly U), polyguanine (poly G), polycytosine (poly C), and poly inosine or polythymidine (if these bases are linked to a ribose sugar). Complementary runs of nucleotides, for example, poly A and poly U, or poly G and poly C, are preferred where a double-stranded hybrid is contemplated.

Irrespective of the overall length of an HDR, the optimal ratio of RNA to DNA may be determined empirically. Although about 5, 10, 15, 20, 25, 50, or even more than 75% DNA is acceptable, it is presently believed that in some embodiments a terminal RNA portion may be substantially larger than the DNA portion without adversely affecting the efficacy of the invention. In other embodiments a terminal RNA portion may be substantially smaller than the DNA portion.

Although optimal sequences for a DNA portion may be determined empirically, at least one portion of an HDR may contain at least one CpG dinucleotide, which may be a CpG sequence, and which may comprise DNA. A “CpG dinucleotide” refers to a nucleic acid sequence having a cytosine followed by a guanine (in 5′ to 3′ orientation) and linked by a phosphate bond. In one embodiment, the pyrimidine ring of the cytosine is unmethylated. Nevertheless, CpG motifs having a methylated cytosine can be effective immunostimulators under certain conditions, (Goeckeritz et al., Internat. Immunol. 11:1693 (1999) (incorporated herein by reference in its entirety)), and thus, CpG motifs as used herein may, but need not necessarily, have an unmethylated cytosine. In further embodiments, HDRs of the invention may comprise multiple CpG motifs which may or may not be separated by RNA nucleotides.

A “CpG sequence” or “CpG motif”,as used herein, refers to CpG dinucleotides, which may be associated with additional DNA sequence or, for the purposes of this invention, RNA sequence, which contributes to immunostimulatory effects. CpG sequences can be determined empirically according to well known techniques in the art, and may be determined or designed according to various canonical formulae, such as those described in U.S. Pat. Nos. 6,194,388, 6,008,200 and 5,856,462, each of which is incorporated herein by reference in its entirety. In one embodiment of the invention, the CpG dinucleotide comprises DNA, but some or all of the remaining bases of the CpG sequence are RNA. In an alternative embodiment, one or both of the CpG dinucleotides comprise RNA. In another embodiment, the CpG sequence is a palindrome. In yet another embodiment, the CpG sequence comprises DNA and forms a palindrome with all or a portion of an RNA portion of the HDR. In one embodiment, the HDR contains a core DNA hexamer having a CpG dinucleotide. In a presently preferred embodiment the CpG dinucleotide is centered in a core DNA hexamer. Representatives of such hexamers include, but are not limited to, GACGTT, TTCGTA, TTCGAG, AGCGTT, CTCGAG, TTCGTT, AGCGTT, MCGTT, AGCGCT, and GTCGGT. In one embodiment, a core DNA hexamer is flanked by RNA. In another embodiment the core DNA hexamer is flanked by between 1 and 5 DNA nucleotides on either or both sides. These flanking DNA sequences may be flanked by RNA. In another embodiment, flanking DNA sequences on either side of the core hexamer are themselves palindromic.

In one embodiment, RNA is added to a pre-existing DNA sequence by enzymatic templated or non-templated polymerization. The added RNA portion may be of any length. Resulting RDRs may be of variable length. In one embodiment, RNA is added to a pre-existing CpG-containing oligonucleotide by non-template directed enzymatic synthesis. The added RNA may be a homopolymer, such as poly A, poly U, or poly I.

HDRs preferably contain one or more CpG dinucleotides which may occur in the context of canonical CpG sequences or motifs. The HDRs of the invention may contain or overlap with a base sequence similar to DNA-based CpG-containing polynucleotides (ODNs) known in the art. Consequently, the hybrid molecules of the invention are useful for the same range of applications as has been suggested for CpG polynucleotides composed entirely of a single sugar backbone (generally deoxyribose). These suggested uses are reviewed in Immunobiology of Bacterial CpG-DNA (Springer, 2000, H. Wagner ed.), which is incorporated herein by reference in its entirety. According to formulae for CpG motifs known in the art, the base sequence of a CpG motif may comprise one or more CpG sequences represented by the formula 5′ N₁N₂MT-CpG-AKN₃N₄ 3′, wherein M is adenine or cytosine; K is guanine or thymidine; and N₁, N₂, N₃, and N₄ are any nucleotides, with the proviso that K is guanine when M is cytosine, and K is thymidine when M is adenine. Thus, an HDR may include a sequence represented by the formula 5′ N₁N₂CT-CpG-AGN₃N₄ 3′ or the formula 5′ N₁N₂AT-CpG-ATN₃N₄3′.

In other embodiments the DNA portion consists of or overlaps with one or more sets of nucleotides of the formula: 5′ N₁X₁CGX₂N₂ 3′, as described in WO 98/37919 (incorporated herein by reference in its entirety). In these embodiments, at least one nucleotide separates consecutive CpGs; where X₁ is adenine, guanine, or thymidine; X2 is cytosine or thymine; N can be absent, can be a single nucleotide or can be a sequence of nucleotides, with the proviso that N₁+N₂ is from 0-26 bases. In this embodiment, it is preferred that N₁ and N₂ do not contain a CCGG quadramer or more than one CGG trimer. The DNAportion is preferably between 8-30 bases, but may be as little as 2-4 bases, preferably including a CpG dinucleotide. Similarly, the DNA portion may consist of or overlap with one or more sets of nucleotides of the formula: 5′ N₁X₁X₂CGX₃X₄N₂ 3′, wherein X₁X₂ is selected from the group consisting of GpT, GpG, GpA, ApT, and ApA, and X₃X₄ is TpT or CpT.

A DNA portion comprising the core hexamer sequence CTCGAG, or NₓCTCGAGNₓ, where Nₓ is one or more DNA nucleotides, will tend to promote a humoral immune response, whereas a DNA portion comprising the CpG sequence ATCGAT or NₓATCGATNₓ, where Nₓ is one or more DNA nucleotides, will tend to promote a cell-mediated immune response. HDRs containing CTCGAG or ATCGAT hexamers comprising RNA or a combination of RNA and DNA may also tend to promote humoral and cell-mediated immune responses, respectively.

Additional factors which should be considered when designing an HDR include the species for which the HDR is to be used. For example, Verthelyi et al., J. of Immunology 166: 2372-77 (2001), (which is incorporated herein by reference in its entirety), teaches that CpG sequences of the formula M₁M₂CGN₁N₂, where M₁ and M₂ are A or G and N₁ and N₂ are T or C appear to be optimal in mice but function poorly in humans. CpG sequences that work well in humans include those of the formula M₁N₁CGM₂N₂, where M₁ and M₂ are A or G and N₁ and N₂ are T or C. These guidlines may also apply to HDRs designed according to the above formula, that is, consisting or comprising the same, or substantially the same base sequence, but having one or more deoxyribose moieties substituted with ribose.

It is also possible to select for ODN sequences which exhibit immunostimulatory specificity. Verthelyi et al. used standard techniques in the art to identify two classes of ODN, designated “D” and “K”. D-class ODNs preferentially stimulate NK cells to secrete IFN-γ, while K-class ODNs preferentially stimulate cell proliferation, activation of monocytes and B cells to secrete IL-6, and production of IgM by B cells. A similar approach can be applied to the HDRs of the invention to identify HDRs which elicit specific immunostimulatory responses.

In one non-limiting example, a known ODN sequence is modified to replace a portion of the deoxyribose backbone with ribose. In another embodiment, one or more ribonucletides are added to the 3′ or 5′ end of the known ODN sequence. Additional embodiments are, of course, evident from the further teachings of this specification.

Modifications and Analogs

The DNA/RNA hybrid polynucleotides of the invention may be synthesized de novo by any techniques known in the art, for example those described in U.S. Pat. No. 5,935,527, (incorporated herein by reference in its entirety), preferably, with any suitable modification which can render the HDR resistant to in vivo degradation resulting from, e.g., exo or endonuclease digestion. For example, the phosphate backbone may be modified by phosphorothioate backbone modification wherein one of the non-bridging oxygens is replaced with sulfur, as set forth in International Patent Application WO 95/26204; U.S. Pat. No. 5,003,097; Stein et al., Nuc. Acids Res. 16(8):3209-21 (1988); Stein, et al., Anal. Biochem. 188:11 (1990); Lyer et al., J. Am. Chem. Soc. 112:1253-54 (1990); and Metelev and Agrawal, Anal. Biochem. 200:342-346 (1992), each of which is incorporated herein by reference in its entirety. Phosphorothioate modifications can occur anywhere in the polynucleotide, preferably at either or both termini, e.g., at least the last two or three 3′ and/or 5′ nucleotides can be liked with phosphorothioate bonds. In one embodiment, all of the RNA bases are linked by phosphorothioate bonds and, alternatively, all nucleotides of the HDR may be linked with phosphorothioate bonds. The HDRs may also be modified to contain a secondary structure (e.g., stem loop structure) such that it is resistant to degradation.

Another modification that renders the RNA and DNA moieties of the HDR less susceptible to degradation is the inclusion of nontraditional bases such as inosine, as well as acetyl-, thio- and similarly modified forms of adenine, cytidine, guanine, thymine, and uridine. Other modified nucleotides include nonionic analogs, such as alkyl or aryl phosphonates (i.e., the charged phosphonate oxygen is replaced with an alkyl or aryl group, as set forth in U.S. Pat. No. 4,469,863, which is incorporated herein by reference in its entirety), phosphodiesters and alkylphosphotriesters (i.e., the charged oxygen atom is alkylated, as set forth in U.S. Pat. No. 5,023,243 and European Patent No. WO 092 574, each of which is incorporated herein by reference in its entirety). Methods for making other DNA backbone modifications and substitutions are described in Uhlmann and Peyman, Chem. Rev. 90:544 (1990); and Goodchild, Bioconjugate Chem. 1:165 (1990), each of which is incorporated herein by reference in its entirety.

HDRs may be ionically or covalently conjugated to appropriate molecules using techniques which are well known in the art, for example, those described by S. S. Wong in Chemistry of Protein Conjugation and Cross-Linking, CRC Press (1991) and Greg T. Hermanson in Bioconjugate Techniques, Academic Press (1996), each of which is incorporated herein by reference in its entirety. Appropriate molecules include high molecular weight molecules such as polysaccharides, poly-L-lysine, carboxymethylcellulose, polyethylene glycol, or polypropylene glycol, haptenic groups, peptides, and antigens. HDRs containing a diol, such as tetraethyleneglycol or hexaethyleneglycol, at either or both termini, may be more resistant to degradation. A variety of coupling or cross-linking agents can be used, e.g., protein A, carbodiimide, and N-succinimidyl-3-(2-pyridyldithio) propionate (SPDP).

Pharmaceutical Compositions

The present invention further provides immunostimulatory compositions comprising one or more HDR sequences alone, or admixed with one or more antigens, moieties, or carriers. The immunostimulatory compositions of the invention may be considered pharmaceutical compositions or, more specifically, immunological compositions in that they elicit a biological effect on the immune system.

An immunostimulatory composition comprising at least one HDR and at least one antigen may be considered immunogenic. As used herein, an antigen is other than an HDR and comprises the following combinations of moieties: 1) at least one T cell epitope, or 2) at least one B cell epitope or, 3) at least one T cell epitope and at least one B cell epitope. Preferably, an immunogenic composition is capable of stimulating an antigen-specific cellular or humoral immune response, preferably characterized by immunologic memory.

In one embodiment, the antigen comprises at least one polynucleotide sequence operationally encoding one or more antigenic polypeptides. Used in this context, the word “comprises” intends that at least one antigenic polypeptide is provided by the transcription and/or translation apparatus of a host cell acting upon an exogenous polynucleotide that encodes at least one antigenic polypeptide, as described, for example in U.S. Pat. Nos. 6,194,389 and 6,214,808.

A vaccine preferably comprises an immunostimulatory composition of the invention associated with, i.e., suspended, dissolved, admixed, adhered, or embedded in, a pharmaceutically acceptable carrier. Moreover, as used herein, a vaccine refers to an immunostimulatory composition comprising one or more HDR sequences for administration to an organism for any prophylactic, ameliorative, palliative, or therapeutic purpose, irrespective of the presence or absence of an antigenic epitope. By way of example, one or more HDRs of the invention in the presence of antigen may comprise a vaccine for the stimulation of specific humoral and/or cellular immunity. Nevertheless, one or more HDRs in the absence of antigen may comprise a vaccine for the stimulation of global or innate immunity.

As used herein, a pharmaceutical composition or vaccine comprises at least one immunological composition, which may be dissolved, suspended, or otherwise associated with a pharmaceutically acceptable carrier or vehicle. Any pharmaceutically acceptable carrier can be employed for administration of the composition. Suitable pharmaceutical carriers are described in Remington's Pharmaceutical Sciences, 18th Edition (A. Gennaro, ed., 1990) Mack Pub., Easton, Pa., which is incorporated herein by reference in its entirety. Carriers can be sterile liquids, such as water, polyethylene glycol, dimethyl sulfoxide (DMSO), oils, including petroleum oil, animal oil, vegetable oil, peanut oil, soybean oil, mineral oil, sesame oil, and the like. Carriers can be in the form of mists, sprays, powders, waxes, creams, suppositories, implants, salves, ointments, patches, poultices, films, or cosmetic preparations.

Proper formulation of the pharmaceutical composition or vaccine is dependent on the route of administration chosen. For example, with intravenous administration by bolus injection or continuous infusion, the compositions are preferably water soluble, and saline is a preferred carrier. For transcutaneous, intranasal, oral, gastric, intravaginal, intrarectal, or other transmucosal administration, penetrants appropriate to the barrier to be permeated may be included in the formulation and are known in the art. For oral administration, the active ingredient may be combined with carriers suitable for inclusion into tablets, pills, dragees, capsules, liquids, gels, syrups, slurries, suspensions, and the like. Time-sensitive delivery systems are also applicable for the administration of the compositions of the invention. Representative systems include polymer base systems such as poly(lactide-glycoside), copolyoxalates, polycaprolactones, polyesteramides, polyorthoesters, polyhydroxybutyric acid and polyanhydrides. These and like polymers may be formulated into microcapsules according to methods known in the art, for example, as taught in U.S. Pat. No. 5,075,109, which is incorporated herein by reference in its entirety. Alternative delivery systems appropriate for the administration of the disclosed immunostimulatory compounds of the invention include those disclosed in U.S. Pat. Nos. 6,194,389, 6,024,983 5,817,637, 6,228,621, 5,804,212, 5,709879, 5,703,055, 5,643,605, 5,643,574, 5,580,563, 5,239,660, 5,204,253, 4,748,043, 4,667,014, 4,452,775, 3,854,480, and 3,832,252 (each of which is incorporated herein by reference in its entirety).

Aqueous dextrose and glycerol solutions can also be employed as liquid carriers, particularly for injectable or aerosol solutions. For administration by aerosol, as by pressurized spray or nebulizer, suitable propellants may be added as understood by those familiar with the art. The immunological composition may also be formulated with solubilizing agents; emulsifiers; stabilizers; dispersants; flavorants; adjuvants; carriers; anesthetics such as bubivaccaine, lidocaine, xylocaine, and the like; antibiotics; and known or suspected anti-viral, anti-fungal, anti-parasitic, or anti-tumor compounds.

Treatment and Administration

The present invention encompasses methods of treating a patient in need of immune stimulation by administering a composition comprising one or more of the HDR sequences of the invention, in the presence or absence of an antigen. As used herein, treatment encompasses corrective, restorative, ameliorative, and preventive methods relating to any disease, condition, abnormality, or symptom. Treatment further encompasses the elicitation or suppression of an immune response in an experimental animal or ex vivo.

Thus, treatment comprises administering an immunostimulatory amount of any of the immunostimulatory compositions of the invention by any method familiar to those of ordinary skill in the art, commonly including oral and intranasal routes, and intravenous, intramuscular, and subcutaneous injections, but also encompassing, intraperitoneal, intracorporeal, intra-articular, intraventricular, intrathecal, topical, tonsillar, mucosal, transdermal, intravaginal, administration and by gavage.

As is recognized by the skilled practitioner, choosing an appropriate administration method may contribute to the efficacy of a treatment, and local administration may be preferred for some applications. Acceptable routes of local administration include subcutaneous, intradermal, intraperitoneal, intravitreal, inhalation or lavage, oral, intranasal, and directed injection into a predetermined tissue, organ, joint, tumor, or cell mass. For example, mucosal application or injection into mucosal lymph nodes or Peyer's patches may promote a humoral immune response with substantial IgA class switching. Alternatively, targeted injection into a lesion, focus, or affected body site may be applicable for the treatment of solid tumors, localized infections, or other situs requiring immune stimulation.

Alternatively, cells of the immune system (e.g., T cells, B cells, NK cells, or oligodendrocytes) may be removed from a host and treated in vitro. The treated cells may be further cultured or reintroduced to a patient (or to a heterologous host) to provide immune stimulation to the patient or host. For example, bone marrow cells may be aspirated from a patient and treated with an HDR to stimulate global or specific immunity. High-dose radiation, or comparable treatments, may then be used to destroy the remaining immune cells in the patient. Upon re-implantation, the autologous HDR-stimulated cells will restore normal immune function in the patient. Alternatively, NK and/or T cells isolated from a patient suffering from cancer may be exposed in vitro to one or more HDRs in the presence of antigens specific to the patient's cancer. Upon re-implantation into the patient, the HDR-stimulated cells will deploy a vigorous cellular immune response against the cancerous cells.

Immunostimulatory Amount

An immunostimulatory (efficacious) amount refers to that amount of vaccine that is able to stimulate an immune response in a patient which is sufficient to prevent, ameliorate, or otherwise treat a pathogenic challenge, allergy, or immunologic abnormality or condition. If co-administered with an antigen of interest, an immunostimulatory amount is that amount which provides a measurable increase in a humoral or cellular immune response to at least one epitope of the antigen as compared to the response obtained if the antigen is administered in the absence of the HDR. Thus, for example, an immunostimulatory amount refers to that amount of an HDR-containing composition that is able to promote the production of antibodies directed against an antigenic epitope of interest or stimulate a detectable protective effect against a pathogenic or allergenic challenge.

Alternatively, if administered to a patient in the presence or absence of antigen, an immunostimulatory amount comprises that amount which stimulates innate immunity. Innate immunity, as noted above, is the ability of an immune system to respond to primary and secondary antigenic challenge and includes the ability to monitor and combat non-malignant tumors, malignant cells, and primary challenge by pathogenic viruses or organisms. Thus, the stimulation of innate immunity encompasses the stimulation of any humoral or cellular immune response, but it is not necessarily related to the co-administration of an antigen. Thus, in this context an immunostimulatory amount is that which is sufficient to prevent or decrease tumor expansion, metastasis, or the morbidity or mortality associated with a pathogenic infection.

Treatment with an immunostimulatory amount of an HDR-containing composition of the invention comprises effecting any directly, indirectly, or statistically observable or measurable increase or other desired change in the immune response in a host, specifically including an ex vivo tissue culture host, comprising at least one cell of the immune system or cell line derived therefrom. Host cells may be derived from human or animal peripheral blood, lymph nodes or the like. Preferred tissue culture hosts include freshly isolated T cells, B cells, macrophages, oligodendrocytes, NK cells, and monocytes, each of which may be isolated or purified using standard techniques. Observable or measurable responses include, B or T cell proliferation or activation; increased antibody secretion; isotype switching; increased cytokine release, particularly the increased release of one or more of IL-1, IL-2, IL-3, IL4, IL-5, IL-6, IL-9, IL-10, IL-12, IL-13, GM-CSF, IFN-γ, TNF-α, TNF-β, GM-CSF, MIP-1α, MIP-1β, or RANTES; increased antibody titer or avidity against a specific antigen; reduced morbidity or mortality rates associated with a pathogenic infection; promoting, inducing, maintaining, or reinforcing viral latency; suppressing or otherwise ameliorating the growth, metastasis, or effects of malignant and non-malignant tumors; and providing prophylactic protection from a disease or the effects of a disease.

Where the suppression of an immunological response is desired, for example, in the treatment of autoimmune disease or allergy, an effective amount also encompasses that amount sufficient to effect a measurable or observable decrease in a response associated with the condition or pathology to be treated.

Immunization Schedule

The amount of an HDR-containing composition to be administered and the frequency of administration can be determined empirically and will take into consideration the age and size of the patient being treated, and the condition or disease to be addressed. An appropriate dose is within the range of 0.01 to 1000 μg, 0.1 to 100 μg, 1 to 50 μg, of HDR per inoculum in a mouse, including any value subsumed within the recited ranges. The amount may be considerably higher in human patients and other larger animals, particularly where a global stimulation of innate immunity is desired. The composition of the invention may be administered continuously by transcutaneous diffusion, intravenous drip, implantable pump, or other suitable delivery system known in the art, preferably in the absence of a target antigen. Where the HDR is administered in the context of a target antigen, an acceptable amount of the target is 0.01 μg to 100 μg per inoculum, but higher and lower amounts may also be indicated. Secondary booster immunizations may be given at intervals ranging from one week to many months later.

HDR Adjuvants and Vaccines

In a preferred embodiment, the HDRs of the invention comprise an adjuvant, defined herein as a composition that promotes or enhances an immune response to a target antigen. Although an adjuvant is not desirably immunogenic, many adjuvants do elicit antibodies. Cholera toxin, for example, elicits a vigorous humoral immune response but, if administered as an adjuvant in conjunction with a target antigen, it also promotes an increased antibody response to epitopes of the target. In contrast, a target antigen is an antigen against which a cellular and/or humoral immune response is desired.

Thus, the hallmark of an adjuvant is the ability to promote an increased humoral or cellular response against at least one epitope not present in the adjuvanting molecule. In one embodiment, this epitope may be expressed on a target antigen administered as a vaccine. In another embodiment, where an HDR-containing composition is administered to boost innate immunity, the target antigen may comprise an epitope of an infectious agent or tumor cell which was not deliberately administered to the patient. In the latter embodiment, as in other embodiments described herein, it is not required that the target be specifically known or identified.

The adjuvants of the present invention all comprise at least one HDR sequence. In one embodiment, the adjuvant is administered in conjunction with at least one target antigen, however, because HDRs globally stimulate the immune response, the adjuvant may be administered within 48 hours, within 24 hours, or within 12 hours of contacting the specific antigen. To maximize the efficacy of treatment, the adjuvant may be administered before or contemporaneously with the target antigen. Thus, the HDR may be co-administered with an antigen, and may be directly or indirectly associated, complexed, or covalently bound to one or more antigenic substance. Methods for covalent conjugation are known in the art and include those described in S.S. Wong, Chemistry of Protein Conjugation and Cross-Linking, CRC Press (1991) and Greg T. Hermanson in Bioconjugate Techniques, Academic Press (1996), each of which is incorporated herein by reference in its entirety.

When the HDR is used as an adjuvant for a target antigen, the antigen of interest may be co-administered with traditional adjuvants (such as alum, Freund's complete and incomplete adjuvants, LPS, cholera toxins, liposomes, BCG, DETOX, Titermax Gold, and the like), as is commonly practiced in the art.

Thus, an adjuvant comprising one or more HDRs can be used to improve the efficacy of any suitable vaccine containing a target antigen. Examples of suitable vaccines can be found in the 54^{th} edition of the Physicians' Desk Reference (2000), which is incorporated herein by reference in its entirety and include those directed against Lyme disease,

Hepatitis A, B, and C, HIV and Malaria.

In addition, appropriate target antigens comprise:

1) proteins, lipoproteins, and glycoproteins, including viral, bacterial, parasitic, animal, and fungal proteins such as albumins, tetanus toxoid, diphtheria toxoid, pertussis toxoid, bacterial outer membrane proteins (including meningococcal outer membrane protein), RSV-F protein, malarial derived peptide, B-lactoglobulin B, aprotinin, ovalbumin, lysozyme, and tumor associated antigens such as carcinoembryonic antigen (CEA), CA 15-3, CA 125, CA 19-9, prostrate specific antigen (PSA), and the TAA complexes of U.S. Pat. No. 5,478,556, which is incorporated herein by reference in its entirety;

2) carbohydrates, including naturally-occurring and synthetic polysaccharides and other polymers such as ficoll, dextran, carboxymethyl cellulose, agarose, polyacrylamide and other acrylic resins, poly (lactide-co-glycolide), polyvinyl alcohol, partially hydrolyzed polyvinyl acetate, polyvinylpryrolidine, Group B Steptococcal and Pneumococcal capsular polysaccharides (including type III), Pseudomonas aeruginosa mucoexopolysaccharide, and capsular polysaccharides (including fisher type I), and Haemophilus influenzae polysaccharides (including PRP);

3) haptens, and other moieties comprising low molecular weight molecules such as TNP, saccharides, oligosaccharides, polysaccharides, peptides, toxins, drugs, chemicals, and allergens; and

4) haptens and antigens derived from bacteria, rickettsiae, fungi, viruses, parasites, including Diphtheria, Pertussis, Tetanus, H. influenzae, S. pneumoniae, E. Coli, Klebsiella, S. aureus, S. epidermidis, N. meningiditis, Polio, Mumps, measles, rubella, Respiratory Syncytial Virus, Rabies, Ebola, Anthrax, Listeria, Hepatitis A, B, C, Human Immunodeficiency Virus I and II, Herpes simplex types 1 and 2, CMV, EBV, Varicella Zoster, Malaria, Tuberculosis, Candida albicans, and other candida, Pneumocystis carinii, Mycoplasma, Influenzae virus A and B, Adenovirus, Group A streptococcus, Group B streptococcus, Pseudomonas aeryinosa, Rhinovirus, Leishmania, Parainfluenzae, types 1, 2 and 3, Coronaviruses, Salmonella, Shigella, Rotavirus, Toxoplasma, Enterovirusses, and Chlamydia trachomatis and pneumoniae.

Moreover, because the HDRs of the invention non-specifically stimulate the immune response independent of the administration of an antigen, the compositions of the present invention can be used to treat, prevent, or ameliorate the symptoms resulting from exposure to a bio-warfare agent. Bio-warfare agents include those naturally occurring biological agents that have been specifically modified in the laboratory. Often, modification of these agents has altered them such that there is no known treatment. Examples include Ebola, Anthrax, and Listeria.

The HDRs of the invention may be administered prior to suspected exposure to a bio-warfare or other infectious agent to globally stimulate the immune system. Such treatment may be particularly efficacious in minimizing the morbidity, mortality, or symptoms associated with a low dose of the infectious agent. In the course of ameliorating the symptoms after exposure, use of the present HDRs may not cure the patient, but rather can extend the patient's life sufficiently such that some other treatment can then be applied.

Similarly, the administration of HDRs to patients traveling may prevent or minimize the effect of contact with unfamiliar infectious agents. In one embodiment, HDR-stimulated innate immunity protects the traveler from parasitic infection.

As suggested above, the immunogenic compositions of the present invention can be used to treat, prevent, or ameliorate any suitable infectious disease, including, but not limited to francisella, schistosomiasis, tuberculosis, AIDS, malaria, sepsis, and leishmania. Examples of suitable infectious viruses, bacteria, fungi, and other organisms (e.g., protists) can be found in International Patent Application WO 98/18810, which is incorporated herein by reference in its entirety. Optionally, the present method can be used in combination with any suitable anti-infectious agent. Suitable anti-infectious agents include those substances given in treatment of the various conditions described elsewhere, examples of which can be found in the Physicians' Desk Reference (2000).

The present inventive method of inducing an immune response can be used to treat, prevent, or ameliorate any allergic reaction. In one embodiment, administration of one or more HDRs in the context of the allergenic antigen stimulates a class switching to non-IgE isotypes. The HDRs and antigen may be co-administered with CD40 ligand, or cytokines such as TGF-β, IL-2, IL-4, and IL-5 as taught in U.S. Pat. No: 5,874,085, which is incorporated herein by reference in its entirety. Optionally, the present inventive method can also be used in combination with any suitable anti-allergenic agent. Suitable antiallergenic agents include those substances given in treatment of the various allergic conditions described above, examples of which can be found in the Physicians' Desk Reference (2000).

An allergy, in the context of the present invention, refers to an acquired hypersensitivity to a substance (i.e., an allergen). Allergic conditions include eczema, allergic rhinitis or coryza, hay fever, bronchial asthma, uticaria (hives), food allergies, and other atopic conditions. The list of allergens is extensive and includes pollens, insect venoms, animal dander, dust fungal spores, and drugs (e.g., penicillin). Additional examples of natural, animal, and plant allergens applicable to the present invention can be found in International Patent Application WO 98/18810, which is incorporated herein by reference in its entirety. In one embodiment, the present inventive method is used to treat allergic asthma.

Administration of the HDRs of the invention can be used to treat any suitable tumor, cancer, or pre-cancerous lesion. Optionally, the present inventive method can be used in combination with any suitable anti-cancer agent. Cancers include cancers of the brain, lung (e.g., small cell and non-small cell), ovary, breast, prostate, and colon, as well as carcinomas and sarcomas. Preferably, the present inventive method is used to treat a solid tumor cancer. Suitable anti-cancer agents include those treatments and substances given in treatment of the various conditions described above including ionizing radiation, specifically targeted cytotoxic compounds, cisplatin-transferrin, fluoxetine, staurosporines, vinblastine, methotrexate, 5-fluorouracil, and leucovorin, further examples of which can be found in the Physicians' Desk Reference (2000).

When employing the HDRs of the present invention as an adjuvant or vaccine component for allergens, haptens, poorly immunogenic peptides, and polysaccharides, the target molecules are preferably conjugated to strong T cell dependent antigens or otherwise complexed to increase their immunogenicity. Haptenic moieties, and other poorly immunogenic molecules, such as polysaccharides may be conjugated to strong T cell dependent antigens or otherwise complexed to increase their immunogenicity, as discussed, for example, by Dick and Bueret in Conjugate Vaccines, Contrib. Microbiol. Immunol. 10:48-114 (1989), Cruse J M and Lewis R E, Jr. eds., which is incorporated herein by reference in its entirety. Moreover, it has recently been shown that conjugation of a T-cell dependent antigen to a poorly immunogenic T cell-independent antigen, (e.g., a polysaccharide) can enhance the immunogenic response to both the T-cell dependent and T-cell independent components. In addition, the antibody response to additional moieties, including poorly immunogenic molecules and haptens (including non-T-cell dependent peptides) can also be dramatically enhanced if further conjugated to the T-cell dependent or T-cell independent carrier, or both, in a “dual conjugate” composition. Lees et al., Vaccine 1160-66 (1994); U.S. Pat. Nos. 5,585,100 and 5,955,079 to Mond and Lees, each of which is incorporated herein by reference in its entirety. This enhanced response is particularly pronounced when B cell epitopes of the additional moieties are intrinsically multivalent or otherwise present in multiple copies, although neither of these properties is absolutely required in the practice of the present invention.

As used herein, a moiety is any substance that is able to stimulate the immune system either by itself or once coupled to an immunogenic molecule. Thus, a moiety comprises an HDR or at least one T or B cell epitope and encompasses haptens, antigens, or combinations thereof. In some embodiments, an HDR is co-administered with, and may be electrostatically or chemically bound as a moiety to an immunogenic dual conjugate composition.

Additional Immunomodulators and Cell Targeting Elements

The immune response elicited by the HDRs and HDR-containing constructs of the invention may be further enhanced by the administration of immunomodulators and/or cell targeting moieties. Where an antigen-specific response is desired, these additional entities are co-administered with, and preferably chemically conjugated to, the antigen or immunogenic composition. Acceptable additional entities (moieties) include, for example, (1) LPS and detoxified lipopolysaccharides or derivatives thereof, (2) muramyl deputies, (3) carbohydrates and lipids (including cationic and anionic lipids, sterols, and the like) that may interact with cell surface determinants to target the construct to immunologically relevant cells; (4) proteins or polypeptides having specific immunological stimulatory activity including, for example, CD40 ligand, and fragments thereof, and polypeptides which bind to the CR2 receptor, including those described in copending U.S. Application No. 09/328,599 entitled: Enhancement of B Cell Activation and Immunoglobulin Secretion by Co-stimulation Of Receptors for Antigen and EBV Gp350/220, filed Jun. 10, 1999, in the names of Drs. James Mond and Andrew Lees, which is incorporated herein by reference in its entirety (5) peptides encoding limitation signals, for example, signals for farnesylation, geranylgeranylation, myristolation, or palmitoylation as described in U.S. Pat. No. 5,776,675, incorporated herein by reference in its entirety; (6) a universal TCE or Pan DR epitope, as described, for example in U.S. Pat. No. 5,114,713 to Sinigaglia; Alexander et al., Immunity 1:751-761 (1994); Ahlborg et al., Infect Immun 68:2102-9 (2000); Kaumaya et. al., J Mol Recognit. 6:81-94 (1993); Greenstein et al., J. Immunol. 148:3970-7 (1992) (each of which is incorporated herein by reference in its entirety); (7) antibodies that interact with cell surface components including, but not limited to, antibodies directed to CR2, CR2 receptors or other components of the antigen receptor complex, CD40 or CD40 ligand, and MHC components; and (8) one or more interleukins, including, but not limited to IL-1, IL-2, IL-3, IL-4, IL-5, IL-6, IL-9, IL-10, IL-12, IL-13, IL-15, GM-CSF, IFN-γ, TNF-α, TNF-β, and GM-CSF, especially combinations of GM-CSF with IL-2, and other immunostimulatory combinations described in copending U.S. application Ser. No. 08/568,343, to Mond and Snapper, filed May 10, 2000, entitled: Compositions For Stimulating The Release of Antibody By B Lymphocytes (which is incorporated herein by reference in its entirety ).

In one embodiment, the immunogenicity of a protein, hapten, or immunogenic composition may be further enhanced by the co-administration of an adjuvanting lipoprotein, as described in the copending U.S. applications Ser. Nos. 09/039,247 and 09/244,773, filed Feb. 5, 1999, and Mar. 16, 1998, respectively, each of which is incorporated herein by reference in its entirety. The lipoprotein may be covalently conjugated to the target protein, hapten, or composition, using, for example the methods described in U.S. Pat. No. 5,693,326 to Lees (incorporated herein by reference in its entirety).

Patient

The invention also relates to the treatment of a host by administration of an immunostimulatory amount of an HDR. A host encompasses both in vivo and ex vivo cells of the immune system, and thus includes the entire range from immortalized or freshly isolated cultured cells through intact organisms having an immune system. Host organisms may be patients, hereby defined as any person or non-human animal in need of immune stimulation, or to any subject for whom treatment may be beneficial, including humans and non-human animals. Such non-human animals to be treated include all domesticated and feral vertebrates, preferably, but not limited to: mice, rats, rabbits, fish, birds, hamsters, dogs, cats, swine, sheep, horses, cattle, and non-human primates.

The present invention is illustrated by the following Examples, which are not intended to be limiting in any way.

### EXAMPLE 1

Oligonucleotide Design Synthesis

Phosphorothioate-substituted oligonucleotides were used to illustrate the surprising and unexpected properties of the RNA/DNA hybrids of the invention. In the following ODN and RDR and RNA sequences, DNA is depicted in capital letters and RNA in lower case.

(SEQ ID NO:1)

| | |
|---|---|
| 5′ AAAAAAAAAAAAAACGTTAAAAAAAAAAAA 3′ | DDD |

(SEQ ID NO:2)

| | |
|---|---|
| 5′ aaaaaaaaaaaaAACGTTaaaaaaaaaaaa 3′ | RDR |

(SEQ ID NO:3)

| | |
|---|---|
| 5′ AAAAAAAAAAAAaacguuAAAAAAAAAAAA 3′ | DRD |

(SEQ ID NO:4)

| | |
|---|---|
| 5′ aaaaaaaaaaaaaacguuaaaaaaaaaaaa 3′ | RRR |

(SEQ ID NO:5)

| | |
|---|---|
| 5′ ggggggggggggAACGTTgggggggggggg 3′ | 75GS |

(SEQ ID NO:6)

| | |
|---|---|
| 5′ aaaaaaaaaaaaCCCGGGaaaaaaaaaaaa 3′ | 74CG |

(SEQ ID NO:7)

| | |
|---|---|
| 5′ aaaaaaaaaaaaaaCGaaaaaaaaaaaaaa 3′ | 7401 |

(SEQ ID NO:8)

| | |
|---|---|
| 5′ GGGGGGGGGGGGaacguuGGGGGGGGGGGG 3′ | 75DNA |

(SEQ ID NO:9)

| | |
|---|---|
| 5′ ctctctctctctaacguuctctctctctct 3′ | 76CT |

(SEQ ID NO:10)

| | |
|---|---|
| 5′ ggggggggggggaacguugggggggggggg 3′ | 75RNA |

(SEQ ID NO:11)

| | |
|---|---|
| 5′ AAAAAAAAAAAAAAGCTTAAAAAAAAAAAA 3′ | DDDC |

The control oligonucleotide, DDD (SEQ ID NO:1), is composed entirely of deoxyribonucleotides. Two representative HDRs, each with a core hexamer sequence identical to that of the control ODN were used in direct comparisons with DDD (SEQ ID NO:1): RDR (SEQ ID NO:2), comprises primarily RNA but contains an internal DNA cassette having the base sequence AACGTT and DRD (SEQ ID NO:3), which is the inverse of RDR, and comprises DNA sequences flanking an internal aaggct sequence of ribonucleotides. RRR (SEQ ID NO:4) comprises the same base sequence of SEQ ID NO:1, but is synthesized entirely from RNA. As noted above, ODN sequences comprised of RNA are widely considered inoperative.

Seven additional ODNs, SEQ ID NOS: 5-11, were generated to assay the relationship between base composition and HDR function.

DDD (SEQ ID NO:1), RDR (SEQ ID NO:2), and DRD (SEQ ID NO:3) were generated on a commercially-available PE/ABI 394 RNA/DNA Synthesizer. DNA precursors were attached at bottle positions 1-4 and RNA precursors, having a protective silyl group for protection of the 2′ position, were attached at the bottle positions 5-8. The remaining bottle positions contained standard chemicals for beta-cyanoethyl diisopropyl phosphoramidite chemistry synthesis, with the exception of bottle No. 15, which contained Beaucage Reagent (1 g/100 ml in acetonitrile) as a sulfurization agent as described in U.S. Pat. No. 5,003,097 (incorporated herein by reference in its entirety). RNA precursors and Beaucage Reagent was purchased from Glen Research of Sterling Va. Acetonitrile was purchased from Burdick & Jackson through VWR Scientific. The remaining chemicals were from PE/ABI (Foster City, Calif.).

The 1 μM Sulfur Synthesis Program provided by PE/ABI is adequate for the preparation of any HDR, as are the general methods provided in Applied Biosystems' User Bulletin 53 and Applied Biosystems Bulletin No. 6: Chemistry for Automated DNA/RNA Synthesis, Mar. 1994. (incorporated herein by reference in its entirety) for EXPEDITE®, PHARMACIA®, and BECKMAN® synthesizers. Nevertheless, a number of modifications were employed to increase yields:

1) The wash step of rinsing the column matrix with acetonitrile was increased by approximately 30%.

2) Capping time was doubled from 5 seconds to 10 seconds.

3) The recommended coupling time of 25 seconds for DNA and 600 seconds for RNA was increased to 725 seconds for all additions.

4) Beaucage Reagent replaced TETD as the sulfurization reagent. Although the usual sulfurization time is 600 seconds for TETD or 20-30 seconds for Beaucage Reagent, sulfurization was extended to 60 seconds with Beaucage.

5) Oligonucleotides were cleaved from the synthesis column matrix using a 3:1 ratio of 30% NH₄OH:ethanol. Exocyclic amine protective groups were removed via heat in the cleavage solution for 18 hours at 55° C. After cooling to room temperature, the oligonucleotides were dried completely in a speed-vac evaporator.

6) The 2′ silyl protective group was removed with 300 μl Tetrabutylammonium Fluoride (TBAF) at room temperature for 22 hours using a test tube rotator to gently agitate the solution.

7) The samples were applied to a PD10 column (Pharmacia/Amersham) to remove the TBAF and other contaminants resulting from synthesis or ammonolysis. Water used for elution was filtered through 2 sterile “stacked” Millex-GV 0.22 μm filters.

8) Oligonucleotides were visualized using PAGE on a 20% polyacrylamide/8M urea gel, which was stained with 1% methylene blue and destained in water.

Oligonucleotide RRR (SEQ ID NO:4) was synthesized using a similar method.

The relative efficacy of the HDRs of the invention may be tested using the standard methods employed in the following Examples. In particular, treatment of the various T cell populations with one or more HDRs will induce the production of Th1 and/or Th2-type cytokines, for example, IFN-γ and IL-6, respectively.

Of course, one of ordinary skill understands that numerous additional in vitro and in vivo assays may also be used to assess the efficacy of a composition within the scope of the invention, as well as the appropriate dosage schedule and an amount sufficient to produce an optimal response. For example, B cell activation may be assessed using methods known in the art (see for example, Liang et a., J. Clin. Invest. 98:1119-29 (1996) (which is incorporated herein by reference in its entirety)). NK activity may be determined as described in WO 98/18810 (which is incorporated herein by reference in its entirety). The effects of HDRs on dendritic cells, macrophages, and monocytes may be determined as described in Stacey et al., J. Immunol. 157:2116 (1996); Chace et al., Clin. Immunol. Immunopathol. 84:185 (1997); Hacker et al., EMBO J. 17:6230 (1998); and Behboudi et al., Immunol. 99:361-66 (2000) (each of which is incorporated herein by reference in its entirety). By comparing the type, amount, and ratios of cytokines and cell surface molecules produced, it will be evident that the HDRs of the invention are useful in stimulating innate and acquired, humoral and cellular immunities. Moreover, one of skill in the art may thereby select the most potent HDR sequences to match the type of immune stimulation desired (Verthelyi et al., J. of Immunology 166: 2372-77 (2001)). Because each HDR will stimulate the immune system in a particular manner (e.g., resulting in a profile of cytokine secretion and/or suppression from one or more T, B, NK, or monocyte populations), it is not only possible to select the most appropriate HDR for a particular type of immune stimulation, but multiple HDRs may be combined to elicit a desired pattern of immune stimulation.

The in vitro assays may be done using human or animal cells (e.g. B, T, NK, oligodendrocytes, or monocytes) isolated according to standard methods in the art. Tester cells may be freshly isolated human peripheral lymphocytes or mouse spleen cells. Depending on the requirements of any particular assay or application, cells may be of mixed population or purified to 99% or greater purity as described in Snapper et al., J. Immunol. 1158:2731-35 (1997) (which is incorporated herein by reference in its entirety). NK cells may be prepared according to Snapper et al., J. Immunol. 151:5212-60 (1993) (which is incorporated herein by reference in its entirety).

Alternatively, previously characterized or established immune cell lines may be employed, for example, B cell lines, or T cell lines, including Th1 cell clones or Th2 cell clones (e.g., AF7 cells).

### EXAMPLE 2

Hybrid DNA/RNA Oligonucleotides Stimulate TH1 and TH2-type Cytokine Production

The stimulation of cytokines IL-6 and IFN-γ in human peripheral lymphocytes cultured from four healthy volunteer subjects, designated S1 through S4, was assayed using standard methods. Briefly, oligonucleotides DDD and RDR of Example 1 were added to the media of cultured cells to final concentrations of 0.3, 3, or 30 μg/ml. 24 hours after oligonucleotide addition, Th1 and Th2-type cytokine levels in the media were determined by ELISA. Results are presented in arbitrary ELISA UNITS (EU) in Table I and Table II below.

TABLE I
Hybrid DNA/RNA Oligonucleotide Stimulates Release of IL-6

| | S1 | S2 | S3 | S4 |
|---|---|---|---|---|
| media control | 0.061 | 0.048 | 0.105 | 0.106 |
| DDD (0.3 μg/ml) | 0.157 | 0.137 | 0.123 | 0.197 |
| (SEQ ID NO: 1) | | | | |
| DDD (3.0 μg/ml) | 0.111 | 0.130 | 0.147 | 0.176 |
| (SEQ ID NO: 1) | | | | |
| DDD (30 μg/ml) | 0.154 | 0.145 | 0.190 | 0.428 |
| (SEQ ID NO: 1) | | | | |
| RDR (0.3 μg/ml) | 0.077 | 0.117 | 0.164 | 0.217 |
| (SEQ ID NO: 2) | | | | |
| RDR (3.0 μg/ml) | 0.656 | 1.168 | 0.692 | 1.023 |
| (SEQ ID NO: 2) | | | | |
| RDR (30 μg/ml) | 1.547 | 1.305 | 1.595 | 1.568 |
| (SEQ ID NO: 2) | | | | |

TABLE II
Hybrid DNA/RNA Oligonucleotide Stimulates Release of IFN-γ

| | S1 | S2 | S3 | S4 |
|---|---|---|---|---|
| media control | 0.061 | 0.048 | 0.105 | 0.106 |
| DDD (0.3 μg/ml) | 0.218 | 0.559 | 0.234 | 0.133 |
| (SEQ ID NO: 1) | | | | |
| DDD (3.0 μg/ml) | 0.279 | 0.447 | 0.249 | 0.158 |
| (SEQ ID NO: 1) | | | | |
| DDD (30 μg/ml) | 0.298 | 0.455 | 0.337 | 0.314 |
| (SEQ ID NO: 1) | | | | |
| RDR (0.3 μg/ml) | 0.25 | 0.558 | 0.237 | 0.153 |
| (SEQ ID NO: 2) | | | | |
| RDR (3.0 μg/ml) | 0.762 | 1.21 | 0.505 | 0.191 |
| (SEQ ID NO: 2) | | | | |
| RDR (30 μg/ml) | 1.592 | 1.198 | 0.792 | 0.492 |
| (SEQ ID NO: 2) | | | | |

As is evident from the results in Table I and Table II, the hybrid DNA/RNA oligonucleotides of the invention stimulate the production of cytokines implicated in eliciting both Th1 (IFN-γ) and Th2 T (IL-6) type responses in human peripheral lymphocytes.

Moreover, a comparison of the results obtained with the hybrid RDR molecule and the DNA control sequence, DDD, reveals the surprising and unexpected superiority of the HDRs of the invention over ODNs. At the highest concentrations tested, for example, the hybrid RDR molecule was 3-fold more effective at inducing IFN-γ and 6-fold more effective at stimulating the release of IL-6. Consequently, it is expected that the HDRs of the invention, including mixtures of HDRs that elicit complementary patterns of activation, will provide correspondingly superior improvement to Th1 and Th2 responses in a patient as compared to DNA-based oligonucleotides.

### EXAMPLE 3

Hybrid DNA/RNA Oligonucleotides Stimulate B Cell Proliferation

The human peripheral B cell populations of Example 2 were assayed for proliferation in the thymidine incorporation assay as described in Brunswick et al., J. Immunol. 140:3364-72 (1988); and Snapper et al., J. Immunol. 155:5582-89 (1995) (each of which is incorporated herein by reference in its entirety). As is evident from the data in Table III, the HDRs of the invention can stimulate a nearly 10-fold increase in B cell replication, as measured by tritiated thymidine incorporation. As shown in Table IV, comparable results were obtained using mouse B cells. Note that the data in Table IV also demonstrate the superiority of oligonucleotide RDR over DRD in this particular assay.

Consequently, administration of HDRs as adjuvants or vaccine components will stimulate the clonal expansion of antigen-specific B cells, thus promoting the production of antibodies and effectively increasing the immunogenicity of a target antigen. In addition, the HDRs will globally stimulate B cells to divide, thereby increasing innate humoral immunity.

TABLE III
Hybrid DNA/RNA Oligonucleotide
Stimulates B Cell Proliferation

| | S1 | S2 | S3 | S4 |
|---|---|---|---|---|
| media control | 364 | 1578 | 864 | 872 |
| DDD (0.3 μg/ml) | 596 | 1646 | 970 | 716 |
| (SEQ ID NO: 1) | | | | |
| DDD (3.0 μg/ml) | 3660 | 15954 | 8926 | 2331 |
| (SEQ ID NO: 1) | | | | |
| DDD (30 μg/ml) | 11571 | 24243 | 28140 | 8378 |
| (SEQ ID NO: 1) | | | | |
| RDR (0.3 μg/ml) | 805 | 3055 | 806 | 1199 |
| (SEQ ID NO: 2) | | | | |
| RDR (3.0 μg/ml) | 2794 | 12397 | 8426 | 3329 |
| (SEQ ID NO: 2) | | | | |
| RDR (30 μg/ml) | 2359 | 3687 | 3434 | 1892 |
| (SEQ ID NO: 2) | | | | |

TABLE IV

| | |
|---|---|
| media control | 131 |
| DDD (30 ug/ml) | 6897 |
| (SEQ ID NO: 1) | |
| DDD (3 ug/ml) | 1998 |
| (SEQ ID NO: 1) | |
| DDD (.3 ug/ml) | 176 |
| (SEQ ID NO: 1) | |
| RDR (30 ug/ml) | 7436 |
| (SEQ ID NO: 2) | |
| RDR (3 ug/ml) | 3924 |
| (SEQ ID NO: 2) | |
| RDR (.3 ug/ml) | 235 |
| (SEQ ID NO: 2) | |
| DRD (30 ug/ml) | 172 |
| (SEQ ID NO: 3) | |
| DRD (3 ug/ml) | 173 |
| (SEQ ID NO: 3) | |
| DRD (.3 ug/ml) | 134 |
| (SEQ ID NO: 3) | |
| 75RNA (30 ug/ml) | na |
| (SEQ ID NO: 10) | |
| 75RNA (3 ug/ml) | 215 |
| (SEQ ID NO: 10) | |
| 75RNA (.3 ug/ml) | 170 |
| (SEQ ID NO: 10) | |
| RRR (30 ug/ml) | 236 |
| (SEQ ID NO: 4) | |
| RRR (3 ug/ml) | 206 |
| (SEQ ID NO: 4) | |
| RRR (.3 ug/ml) | 160 |
| (SEQ ID NO: 4) | |

na: not available

### EXAMPLE 4

Hybrid DNA/RNA Oligonucleotides Stimulate Antibody Secretion

The ability of HDRs to activate B cells to produce antibody was illustrated using the polyclonal activation and ELISA assays essentially as described in Pecanha et al., J. Immunol. 146:883-89 (1991); and Snapper et al., J. Immunol. 154:5842-50 (1995) (each of which is incorporated herein by reference in its entirety). The techniques described in Finkelman et al., J. Immunol. 138:2826-30 (1987) (which is incorporated herein by reference in its entirety), are also appropriate. In addition, methods for assaying for the stimulation of antibody production and class switching, especially IgA class switching, are evident from U.S. Pat. No. 5,874,085 to Mond and Snapper, which is incorporated herein by reference in its entirety.

TABLE V
IgM Secretion From Human Peripheral B Cells (in μg/ml)

| | S1 | S2 | S3 | S4 |
|---|---|---|---|---|
| media control | 0.584 | 0.455 | 0.574 | 0.461 |
| DDD (0.3 μg/ml) | 0.652 | 0.470 | 0.583 | 0.446 |
| (SEQ ID NO: 1) | | | | |
| DDD (3.0 μg/ml) | 1.031 | 0.772 | 1.003 | 0.5 |
| (SEQ ID NO: 1) | | | | |
| DDD (30 μg/ml) | 1.523 | 0.650 | 1.745 | 0.647 |
| (SEQ ID NO: 1) | | | | |
| RDR (0.3 μg/ml) | 0.556 | 0.450 | 0.584 | 0.437 |
| (SEQ ID NO: 2) | | | | |
| RDR (3.0 μg/ml) | 0.702 | 0.470 | 0.743 | 0.444 |
| (SEQ ID NO: 2) | | | | |
| RDR (30 μg/ml) | 0.507 | 0.395 | 0.506 | 0.45 |
| (SEQ ID NO: 2) | | | | |

As shown in Table V, the RDR oligonucleotide did not elicit antibody secretion substantially above background in this particular experiment (values are in arbitrary ELISA units). This lack of effect may be due to experimental error, or a lack of sensitivity of the assay. Nevertheless, in a subsequent experiment shown in Table VI, purified human peripheral B cells secreted up to 22-fold more antibody following exposure to the RDR oligonucleotide.

TABLE VI
IgM Secretion From Purified Human Peripheral B Cells
(in μg/ml)

| | P1 | P2 | P3 |
|---|---|---|---|
| media control | 15 | 9 | 25 |
| DDD (0.3 μg/ml) | 14 | 8 | 30 |
| (SEQ ID NO: 1) | | | |
| DDD (3.0 μg/ml) | 33 | 20 | 80 |
| (SEQ ID NO: 1) | | | |
| DDD(30 μg/ml) | 100 | 160 | 1500 |
| (SEQ ID NO: 1) | | | |
| RDR (0.3 μg/ml) | 12 | 9 | 23 |
| (SEQ ID NO: 2) | | | |
| RDR (3.0 μg/ml) | 38 | 23 | 90 |
| (SEQ ID NO: 2) | | | |
| RDR (30 μg/ml) | 215 | 48 | 550 |
| (SEQ ID NO: 2) | | | |

### EXAMPLE 5

Hybrid DNA/RNA Oligonucleotides Stimulate Individual T Cells to Secrete Th1-type and Th2-type Cytokines

DBA/2 mouse spleen cells were treated with medium, or medium containing 3.0 μg/ml of RDR or control oligonucleotides. The cells were then subject to an enzyme-linked immunospot (ELISPOT) assay to identify cells expressing IL-6, IL-10, IL-12, and IFN-γ. Table VII reports the number of positive cells per 100,000 cells. ELISPOT assays are well known in the art. Representative methods are described in Czerkinsky et al., J. Immunol. Meth. 65:109-121 (1983); Sedgwich and Holt, J. Immunol. Meth. 57:301-309 (1983); Amano et al., J. Immunol. Meth. 144:127-140; Sparholt et al., Clin. Exp. Allergy, 21:85-90 (1991); and Jones et al., Autoimmunity, 31:117-124 (1999), each of which is incorporated herein by reference in its entirety. As is recognized by one of ordinary skill, the ELISPOT method may be modified to use any T cell type, subtype, or established T cell tester line. Moreover, antibodies directed against any relevant cytokine may be used to test the efficacy of a particular HDR to be assayed.

TABLE VII
Hybrid DNA/RNA Oligonucleotide Stimulates Substantially
More Th1 and Th2 Cells Than A Corresponding DNA-based
Adjuvant

| | IL-6 | IL-10 | IL-12 | IFN-γ |
|---|---|---|---|---|
| medium | 144 | 128 | 144 | 109 |
| DDD (SEQ ID NO: 1) | 256 | 3 | 256 | 52 |
| RRR (SEQ ID NO: 4) | 160 | 1 | 235 | 5 |
| RDR (SEQ ID NO: 2) | 8976 | 3 | 2564 | 140 |

The control ODN, DDD (SEQ ID NO.1), provides to a roughly ⅔-fold increase in the number of T cells expressing IL-6 (a Th1-type cytokine) and IL-12 (a Th2-type cytokine). DDD also reduced by half the number of cells expressing IFN-γ and substantially reduced IL-10 production. As expected, the RNA-based oligonucleotide did not stimulate IL-6 production. Interestingly, it did induce some cells to secrete IL-12 and virtually ablated IL-10 and IFN-γ expression. These results are essentially consistent with the view that RNA-based adjuvants are clinically irrelevant.

In surprising contrast to the effects of single-sugar constructs, the RNA/DNA hybrid of the invention, RDR (SEQ ID NO:2), did not reduce (and, in fact, increased) the number of cells expressing IFN-γ and dramatically increased the proportion of cells secreting both IL-6 and IL-12. Indeed, as compared with the DDD control of the same base sequence, treatment with the HDR construct induced 10-fold more cells to secrete IL-12, and fully 35-fold more cells to express IL-6. This dramatic and unexpected increase in the number of responsive T cells is indicative of the clinical advantage enjoyed by the compositions of the invention in stimulating humoral and cellular immune responses.

### EXAMPLE 6

Dose-response Study of DDD and DRD Oligonucleotide Adjuvants

Table VIII presents the results of a dose-response experiment performed essentially as described for Example 5. Briefly, these results confirm the superiority of the RNA:DNA hybrids of the invention in stimulating cells of the immune system to secrete IL-6 and IL-12. (Data are number of positive cells per 100,000.) This effect is most pronounced at higher nucleotide concentrations, suggesting that local concentrations in excess of 3 μg/ml may be most efficacious. Curiously, the RDR (SEQ ID NO:2) and the DDD (SEQ ID NO:1) control were roughly equally stimulatory of IFN-γ production at the higher concentrations tested.

TABLE VIII

| | IL-6 | IL-12 | IFN-γ |
|---|---|---|---|
| medium | 1292 | 657 | 168 |
| DDD (3.0 μg/ml) | 8750 | 2195 | 688 |
| (SEQ ID NO: 1) | | | |
| DDD (0.3 μg/ml) | 3798 | 2035 | 360 |
| (SEQ ID NO: 1) | | | |
| DDD (0.03 μg/ml) | 2083 | 962 | 176 |
| (SEQ ID NO: 1) | | | |
| DDD (0.003 μg/ml) | 1522 | 652 | 72 |
| (SEQ ID NO: 1) | | | |
| DDD (0.0003 μg/ml) | 1387 | 737 | 136 |
| (SEQ ID NO: 1) | | | |
| RDR (3.0 μg/ml) | 11250 | 3397 | 552 |
| (SEQ ID NO: 2) | | | |
| RDR (0.3 μg/ml) | 3990 | 3237 | 544 |
| (SEQ ID NO: 2) | | | |
| RDR (0.03 μg/ml) | 1410 | 1186 | 232 |
| (SEQ ID NO: 2) | | | |
| RDR (0.003 μg/ml) | 833 | 625 | 144 |
| (SEQ ID NO: 2) | | | |
| RDR (0.0003 μg/ml) | 978 | 545 | 232 |
| (SEQ ID NO: 2) | | | |

### EXAMPLE 7

HDR Function is Related to Structure

The activity of ODNs is known to vary with sequence. To assess whether HDR activity also varies based on sequence a number of different HDRs were designed and tested for their ability to stimulate individual T cells to secrete Th1-type and Th2-type cytokines. This experiment was performed similarly to the one described in Example 5, with the exception that human PBLs were used. As shown in Table IX, the ability of HDRs to stimulate Th1-type and Th2-type cytokine production is highly dependent on HDR sequence. (Data are number of positive cells per 100,000.) HDRs can thus be designed to preferentially stimulate Th-1 vs. Th-2 type responses. Moreover, HDRs eliciting different, even complimentary, patterns of cytokine stimulation can be used in concert to stimulate a desired immune response.

TABLE IX

| | IL-6 | IL-12 |
|---|---|---|
| media control | 1944 | 408 |
| DDD (30 ug/ml) | 1215 | 918 |
| (SEQ ID NO: 1) | | |
| DDD (30 ug/ml) | 1944 | 1122 |
| (SEQ ID NO: 1) | | |
| RDR (30 ug/ml) | 3159 | 1326 |
| (SEQ ID NO: 2) | | |
| DRD (30 ug/ml) | 2552 | 765 |
| (SEQ ID NO: 3) | | |
| 74CG (30 ug/ml) | 2066 | 618 |
| (SEQ ID NO: 6) | | |
| 74C1 (15 ug/ml) | 4860 | 9 |
| (SEQ ID NO: 7) | | |
| 74C1 (30 ug/ml) | 2309 | 8 |
| (SEQ ID NO: 7) | | |
| 75DNA (30 ug/ml) | 3281 | 765 |
| (SEQ ID NO: 8) | | |
| 76CT (15 ug/ml) | 4253 | 2040 |
| (SEQ ID NO: 9) | | |
| 76CT (26.7 ug/ml) | 2187 | 10 |
| (SEQ ID NO: 9) | | |

### EXAMPLE 8

HDRs Stimulate Innate Immunity in vivo

An HDR is suspended in phosphate buffered saline and injected intraperitoneally into DBA/2 mice at a dose of 2-500 μg/animal. Twenty-four hours later spleen cells from some of the injected mice and mock-injected PBS controls are analyzed for expression of B cell surface activation markers Ly-6A/E, Bla-1, and class II MHC, using three-color flow cytometry, and for spontaneous proliferation activity using a standard tritiated thymidine assay. Expression of activation markers will be significantly increased in the HDR injected mice as opposed to the controls. Similarly, cells from the HDR injected animals will incorporate significantly more labeled thymidine. Samples of spleen cells from injected mice are analyzed for NK activity using, for example, the short term chromium release assay described by Ballas et al., J. Immunol. 150:17 (1993) (which is incorporated herein by reference in its entirety). Cells from HDR injected animals will show increased levels of NK cell activation as compared to controls.

Four days after injection, serum is collected from the remaining mice and analyzed for total IgM by ELISA or Octerlony assay. HDR injected mice will show increased levels of total IgM as opposed to the PBS injected controls.

### EXAMPLE 9

HDRs Stimulate Innate Immunity in vivo

A single administration of a CpG ODN can confer immune protection against L. monocytogenes infection in mice that lasts for up to two weeks (Krieg et al., J. of Immunology, 161: 2428-2434 (1998)) (incorporated herein by reference in its entirety). If the ODN is administration in repeated this resistance can be maintained indefinetly (Kinman et al., Infection and Immunity, 67: 5658-63 (1999)) (incorporated herein by reference in its entirety).

To demostrate that the HDRs of the invention are similarly capable of stimulating innate immunity, we employed the technique described in Klinman et al., Infection and Immunity, 67: 5658-63 (1999), which assays resistance to bacterial challenge. Briefly, BALB/c mice were injected with various agents (as described in Table X) and challenged 5 days later with 1,000 LD 50's of L. monocytogenes. As seen in Table X, a single administration of either DDD (SEQ ID NO:1) or RDR (SEQ ID NO:2) (a representative example of an HDR) is capable of conferring resistance in most or all of the mice tested for, at a minimum, 5 days following administration. As expected, the effects of the oligonucleotides could not be demonstrated by 4 weeks post-administration. As demonstrated in Table X, administeration of the ODN in the context of a liposome, which significantly extends the period over which stimulatory material is released, extends the period of detectable increased innate immunity to at least 4 weeks. Administration of HDRs of the invention in the context of liposomes, depot adjuvants such as alum, cochleates, conjugates, linkage to large polymers such as polyethylene glycol (PEGylation), time sensitive delivery formulation, or other forms which delay the release or degradation of the HDR will also extend the period of immune stimulation, as will repeated administrations of the stimulatory HDR.

TABLE X

| | 5 days | 4 weeks |
|---|---|---|
| saline control | 0/5 | 0/5 |
| DDD (SEQ ID NO: 1) | 5/5 | 0/5 |
| RDR (SEQ ID NO: 2) | 4/5 | 0/5 |
| DDDC (SEQ ID NO: 11) | 1/5 | 0/5 |
| DDD (SEQ ID NO: 1) - | 5/5 | 5/5 |
| liposome | | |
| DDD (SEQ ID NO: 1) - | 0/5 | 0/5 |
| liposome | | |

### EXAMPLE 10

HDRs Stimulate Antibody Production and Class Switching in vivo

An HDR is suspended in phosphate buffered saline along with bovine serum albumin (BSA). A dose comprising approximately 2-500 μg of oligonucleotide and 1-25 μg of protein is injected subcutaneously into Balb/c mice. Control mice are injected with a corresponding dose of protein without nucleotide. Additional groups of mice co-injected with protein, or protein plus HDR, are coinjected with GM-CSF, and GM-CSF and IL-2, or other cytokines and cytokine combinations. Injections are repeated after 14 days.

Serum collected two weeks later is tested by ELISA for antibodies reactive against the target antigen. ELISA assays are also used to determine the relative, or preferably, the absolute level of anti-BSA antibodies of each isotype. HDR injected animals will show elevated levels of anti-BSA antibody, in particular increased levels of IgA and/or IgG antibodies, and may show increased levels of IgG₁, IgG₂, and/or IgG₂ₐ isotypes.

### EXAMPLE 11

Representative HDRs of the Invention

The following HDRs are representative of the invention and not limiting in any way. These illustrative sequences have been selected in light of ODN sequences known in the art to posses immunostimulatory activity (innate, global, cellular and/or humoral), and in light of the surprising observation reported herein that hybrid RNA-DNA ONDs (HDRs) possess robust immunostimulary activity both in vitro an in vivo. Using the teachings of Examples 1-10, or other assays commonly used in the art, the skilled artisan will recognize that such HDRs, and all other HDR sequences within the scope of the invention can be assayed in vitro or in vivo for immunostimulatory activity.

In the following sequences, “t” refers to thymidine linked to at least one other base through a ribose sugar. There present invention further contemplates HDRs wherein any “u” (uracil) replaces any “t”, and, further, where “i” (inosine linked to at least one other base through a ribose sugar), replaces any ribose-linked base in the following exemplary sequences.

| | |
|---|---|
| TCAACGTTaacgtt | (SEQ ID NO:12) |
| TCCATGACGTTCCTGATGCTaacgtt | (SEQ ID NO:13) |
| ATCGACTCTCGAGCGTTCTCaacgtt | (SEQ ID NO:14) |
| GCATGACGTTGAGCTaacgtt | (SEQ ID NO:15) |
| TCAGCGCTaacgtt | (SEQ ID NO:16) |
| GAGAACGCTGGACCTTCCATaacgtt | (SEQ ID NO:17) |
| GAGAACGCTCGACCTTCCATaacgtt | (SEQ ID NO:18) |
| GAGAACGCTCGACCTTCGATaacgtt | (SEQ ID NO:19) |
| GAGAACGCTCCAGCACTGATaacgtt | (SEQ ID NO:20) |
| TCCATGTCGGTCCTGATGCTaacgtt | (SEQ ID NO:21) |
| TCCATGTCGGTCCTGCTGATaacgtt | (SEQ ID NO:22) |
| ATGGACTCTCCAGCGTTCTCaacgtt | (SEQ ID NO:23) |
| ATGGAAGGTCCAACGTTCTCaacgtt | (SEQ ID NO:24) |
| gctagacGTTAGCGT | (SEQ ID NO:25) |
| tcaacGTT | (SEQ ID NO:26) |
| tccatgacGTTCCTGATGCT | (SEQ ID NO:27) |
| atcgactctcGAGCGTTCTC | (SEQ ID NO:28) |
| gcatgacGTTGAGCT | (SEQ ID NO:29) |
| tcagcGCT | (SEQ ID NO:30) |
| gagaacGCTGGACCTTCCAT | (SEQ ID NO:31) |
| gagaacGCTCGACCTTCCAT | (SEQ ID NO:32) |
| gagaacgctcGACCTTCGAT | (SEQ ID NO:33) |
| gagaacGCTCCAGCACTGAT | (SEQ ID NO:34) |
| tccatgtcGGTCCTGATGCT | (SEQ ID NO:35) |
| tccatgtcGGTCCTGCTGAT | (SEQ ID NO:36) |
| atggactctccagcGTTCTC | (SEQ ID NO:37) |
| atggaaggtccaacGTTCTC | (SEQ ID NO:38) |
| tccatggcGGTCCTGATGCT | (SEQ ID NO:39) |
| tccatgacGGTCCTGATGCT | (SEQ ID NO:40) |
| tccatgtcGATCCTGATGCT | (SEQ ID NO:41) |
| tccatgtcGCTCCTGATGCT | (SEQ ID NO:42) |
| tccatgtcGTTCCTGATGCT | (SEQ ID NO:43) |
| tccataacGTTCCTGATGCT | (SEQ ID NO:44) |
| tccatgacGTCCCTGATGCT | (SEQ ID NO:45) |
| TCCATGGCGGTCCTGATGCTaacgtt | (SEQ ID NO:46) |
| TCCATGACGGTCCTGATGCTaacgtt | (SEQ ID NO:47) |
| TCCATGTCGATCCTGATGCTaacgtt | (SEQ ID NO:48) |
| TCCATGTCGCTCCTGATGCTaacgtt | (SEQ ID NO:49) |
| TCCATGTCGTTCCTGATGCTaacgtt | (SEQ ID NO:50) |
| TCCATAACGTTCCTGATGCTaacgtt | (SEQ ID NO:51) |
| TCCATGACGTCCCTGATGCTaacgtt | (SEQ ID NO:52) |
| GCTAGACGTTAGCGTacaacgtt | (SEQ ID NO:53) |
| TCAACGTTacaacgtt | (SEQ ID NO:54) |
| TCCATGACGTTCCTGATGCTacaacgtt | (SEQ ID NO:55) |
| ATCGACTCTCGAGCGTTCTCacaacgtt | (SEQ ID NO:56) |
| GCATGACGTTGAGCTacaacgtt | (SEQ ID NO:57) |
| TCAGCGCTacaacgtt | (SEQ ID NO:58) |
| GAGAACGCTGGACCTTCCATacaacgtt | (SEQ ID NO:59) |
| GAGAACGCTCGACCTTCCATacaacgtt | (SEQ ID NO:60) |
| GAGAACGCTCGACCTTCGATacaacgtt | (SEQ ID NO:61) |
| GAGAACGCTCCAGCACTGATacaacgtt | (SEQ ID NO:62) |
| TCCATGTCGGTCCTGATGCTacaacgtt | (SEQ ID NO:63) |
| TCCATGTCGGTCCTGCTGATacaacgtt | (SEQ ID NO:64) |
| ATGGACTCTCCAGCGTTCTCacaacgtt | (SEQ ID NO:65) |
| ATGGAGGTCCAACGTTCTCacaacgtt | (SEQ ID NO:66) |
| TCCATGGCGGTCCTGATGCTacaacgtt | (SEQ ID NO:67) |
| TCCATGACGGTCCTGATGCTacaacgtt | (SEQ ID NO:68) |
| TCCATGTCGATCCTGATGCTacaacgtt | (SEQ ID NO:69) |
| TCCATGTCGCTCCTGATGCTacaacgtt | (SEQ ID NO:70) |
| TCCATGTCGTTCCTGATGCTacaacgtt | (SEQ ID NO:71) |
| acaacgttGCTAGACGTTAGCGT | (SEQ ID NO:72) |
| acaacgttTCAACGTT | (SEQ ID NO:73) |
| acaacgttTCCATGACGTTCCTGATGCT | (SEQ ID NO:74) |
| acaacgttATCGACTCTCGAGCGCGCTC | (SEQ ID NO:75) |
| acaacgttGCATGACGTTGAGCT | (SEQ ID NO:76) |
| accaacgttTCAGCGCT | (SEQ ID NO:77) |
| acaacgttGAGAACGCTGGACCTTCCAT | (SEQ ID NO:78) |
| acaacgttGAGAACGCTCGACCTTCCAT | (SEQ ID NO:79) |
| acaacgttGAGAAOGCTCGACCTTCGAT | (SEQ ID NO:80) |
| acaacgttGAGAACGCTCCAGCACTGAT | (SEQ ID NO:81) |
| acaacgttTCCATGTCGGTCCTGATGCT | (SEQ ID NO:82) |
| acaacgttTCCATGTCGGTCCTGCTGAT | (SEQ ID NO:83) |
| acaacgttATGGACTCTCCAGCGTTCTC | (SEQ ID NO:84) |
| acaacgttATGGAAGGTCCAACGTTCTC | (SEQ ID NO:85) |
| acaacgttTCCATGGCGGTCCTGATGCT | (SEQ ID NO:86) |
| acaacgttTCCATGACGGTCCTGATGCT | (SEQ ID NO:87) |
| acaacgttTCCATGTCGATCCTGATGCT | (SEQ ID NO:88) |
| acaacgttTCCATGTCGCTCCTGATGCT | (SEQ ID NO:89) |
| acaacgttTCCATGTCGTTCCTGATGCT | (SEQ ID NO:90) |
| GCTAgacgttAGCGT | (SEQ ID NO:91) |
| TCAAcgTT | (SEQ ID NO:92) |
| TCCATgacgttCCTGATGCT | (SEQ ID NO:93) |
| ATCGACTctcgagcgttCTC | (SEQ ID NO:94) |
| GCATgacgttGAGCT | (SEQ ID NO:95) |
| TCAGcgCT | (SEQ ID NO:96) |
| GAGaacgctGGACCTTCCAT | (SEQ ID NO:97) |
| GAGAACGctcgacCTTCCAT | (SEQ ID NO:98) |
| GAGAAcgctcgacCTTCGAT | (SEQ ID NO:99) |
| GAGAAcgCTCCAGCACTGAT | (SEQ ID NO:100) |
| TCCATgtcggtCCTGATGCT | (SEQ ID NO:101) |
| TCCATgtcggtCCTGCTGAT | (SEQ ID NO:102) |
| ATGGACtctccaGCGTTCTC | (SEQ ID NO:103) |
| ATGGAAggtccaaCGTTCTC | (SEQ ID NO:104) |
| TCCATggcgGTCCTGATGCT | (SEQ ID NO:105) |
| TCCATGacggtccTGATGCT | (SEQ ID NO:106) |
| TCCATGTcgatCCTGATGCT | (SEQ ID NO:107) |
| TCCATGtcgctccTGATGCT | (SEQ ID NO:108) |
| TCCAtgtcgTTCCTGATGCT | (SEQ ID NO:109) |
| TCCATAAcgTTCGTGATGCT | (SEQ ID NO:110) |
| TCCAtgacgtccctgatGCT | (SEQ ID NO:111) |
| gctaGACGTTagcgt | (SEQ ID NO:112) |
| tcAACGTT | (SEQ ID NO:113) |
| tccatGACGTTcctgatgct | (SEQ ID NO:114) |
| atcgactCTCGAGcgttctc | (SEQ ID NO:115) |
| gcatGACGTTgagct | (SEQ ID NO:116) |
| tcAGCGCT | (SEQ ID NO:117) |
| gagAACGCTggaccttccat | (SEQ ID NO:118) |
| gagaacgCTCGACcttccat | (SEQ ID NO:119) |
| gagaacgCTCGACcttcgat | (SEQ ID NO:120) |
| gagaacgctcCAGCACtgat | (SEQ ID NO:121) |
| gagaACGCTCcagcactgat | (SEQ ID NO:122) |
| gagaACGCTCCAGCACtgat | (SEQ ID NO:123) |
| tccatGTCGGTcctgatgct | (SEQ ID NO:124) |
| tccatGTCGGTcctgctgat | (SEQ ID NO:125) |
| atggactctccAGCGTTctc | (SEQ ID NO:126) |
| atggaaggtccAACGTTCtc | (SEQ ID NO:127) |
| tccatGGCGGTcctgatgct | (SEQ ID NO:128) |
| tccatGACGGTcctgatgct | (SEQ ID NO:129) |
| tccatGTCGATcctgatgct | (SEQ ID NO:130) |
| tccatGTCGCTcctgatgct | (SEQ ID NO:131) |
| tccatGTCGTTcctgatgct | (SEQ ID NO:132) |
| tccatAACGTTcctgatgct | (SEQ ID NO:133) |
| tccatGACGTCcctgatgct | (SEQ ID NO:134) |
| GCTAGACGTTagcgt | (SEQ ID NO:135) |
| TCAACGTTTCACGTaaaa | (SEQ ID NO:136) |
| aaaaTCAACGTTTCACGT | (SEQ ID NO:137) |
| TCCATGACGTTcctgatgct | (SEQ ID NO:138) |
| ATCGACTCTCGagcgttctC | (SEQ ID NO:139) |
| GCATGACGTTgagct | (SEQ ID NO:140) |
| TCAGCgct | (SEQ ID NO:141) |
| tcAGCGct | (SEQ ID NO:142) |
| GAGAACGCTGgaccttccat | (SEQ ID NO:143) |
| GAGAACGCTCGACcttccat | (SEQ ID NO:144) |
| GAGAACGCTCGACcttcgat | (SEQ ID NO:145) |
| GAGAACGctccagcactgat | (SEQ ID NO:146) |
| GAGAACGCTCcagcactgat | (SEQ ID NO:147) |
| GAGAACGCTCCAGCactgat | (SEQ ID NO:148) |
| GAGAACGCTCCAGCACtgat | (SEQ ID NO:149) |
| GAGAACGCTCCAGCACTGAttttttt | (SEQ ID NO:150) |
| GAGAACGCTCCAGCACTGaaaaaaa | (SEQ ID NO:151) |
| TCCATGTCGgtcctgatgct | (SEQ ID NO:152) |
| TCCATGTCGGTcctgctgat | (SEQ ID NO:153) |
| ATGGACTCTCCAGCGTtctc | (SEQ ID NO:154) |
| ATGGAAGGTCCAACGTTctc | (SEQ ID NO:155) |
| TCCATGGCGGTcctgatgct | (SEQ ID NO:156) |
| TCCATGACGGTcctgatgct | (SEQ ID NO:157) |
| TCCATGTCGATcctgatgct | (SEQ ID NO:158) |
| TCCATGTCGCTcctgatgct | (SEQ ID NO:159) |
| TCCATGTCGTTcctgatgct | (SEQ ID NO:160) |
| TCCATAACGTTcctgatgct | (SEQ ID NO:161) |
| TCCATGACGTCcctgatgct | (SEQ ID NO:162) |
| gctagaCGTTAGCGT | (SEQ ID NO:163) |
| tcaaCGTT | (SEQ ID NO:164) |
| tccatGACGTTCCTGATGCT | (SEQ ID NO:165) |
| atcgactCTCGAGCGTTCTC | (SEQ ID NO:166) |
| gcatGACGTTGAGCT | (SEQ ID NO:167) |
| tcagCGCT | (SEQ ID NO:168) |
| gagAACGGTGGACCTTCCAT | (SEQ ID NO:169) |
| gagAACGCTCGACCTTCCAT | (SEQ ID NO:170) |
| gagAACGCTCGACCTTCGAT | (SEQ ID NO:171) |
| gagAACGCTCCAGCACTGAT | (SEQ ID NO:172) |
| tccatGTCGGTCCTGATGCT | (SEQ ID NO:173) |
| tccatGTCGGTCCTGCTGAT | (SEQ ID NO:174) |
| atggactctcCAGCGTTCTC | (SEQ ID NO:175) |
| atggaaggtccAACGTTCTC | (SEQ ID NO:176) |
| tccaTGGCGGTCCTGATGCT | (SEQ ID NO:177) |
| tccatGACGGTCCTGATGCT | (SEQ ID NO:178) |
| tccatGTCGATCCTGATGCT | (SEQ ID NO:179) |
| tccatgTCGCTCCTGATGCT | (SEQ ID NO:180) |
| tccatGTCGTTCCTGATGCT | (SEQ ID NO:181) |
| tccatAACGTTCCTGATGCT | (SEQ ID NO:182) |
| tccatGACGTCCCTGATGCT | (SEQ ID NO:183) |
| gctagaCGttagcgt | (SEQ ID NO:184) |
| tcAACGtt | (SEQ ID NO:185) |
| tccatgaCGttcctgatgct | (SEQ ID NO:186) |
| atcgactctCGagcgttctc | (SEQ ID NO:187) |
| gcatgaCGttgagct | (SEQ ID NO:188) |
| tcagCGct | (SEQ ID NO:189) |
| gagaaCGctggaccttccat | (SEQ ID NO:190) |
| gagaaCGctcgaccttccat | (SEQ ID NO:191) |
| gagaaCGctCGaccttccat | (SEQ ID NO:192) |
| gagaacgctCGaccttccat | (SEQ ID NO:193) |
| gagaaCGctcgaccttcgat | (SEQ ID NO:194) |
| gagaacgctCGaccttcgat | (SEQ ID NO:195) |
| gagaaCGctCGaccttcgat | (SEQ ID NO:196) |
| gagaaCGctccagcactgat | (SEQ ID NO:197) |
| tccatgtCGgtcctgatgct | (SEQ ID NO:198) |
| tccatgtCGgtcctgctgat | (SEQ ID NO:199) |
| atggactctccagCGttctc | (SEQ ID NO:200) |
| atggaaggtccaaCGttctc | (SEQ ID NO:201) |
| tccatggCGgtcctgatgct | (SEQ ID NO:202) |
| tccatgaCGgtcctgatgct | (SEQ ID NO:203) |
| tccatgtCGatcctgatgct | (SEQ ID NO:204) |
| tccatgtCGctcctgatgct | (SEQ ID NO:205) |
| tccatgtCGttcctgatgct | (SEQ ID NO:206) |
| tccataaCGttcctgatgct | (SEQ ID NO:207) |
| tccatgaCGtccctgatgct | (SEQ ID NO:208) |
| GCTAGACGTTAGCGTttttt | (SEQ ID NO:209) |
| TCAACGTTttttt | (SEQ ID NO:210) |
| TCCATGACGTTCCTGATGCTttttt | (SEQ ID NO:211) |
| ATCGACTCTCGAGCGTTCTCttttt | (SEQ ID NO:212) |
| GCATGACGTTGAGCTttttt | (SEQ ID NO:213) |
| TCAGCGCTttttt | (SEQ ID NO:214) |
| GAGAACGCTGGACCTTCCATttttt | (SEQ ID NO:215) |
| GAGAACGCTCGACCTTCCATttttt | (SEQ ID NO:216) |
| GAGAACGCTCGAOCTTCGAtttttt | (SEQ ID NO:217) |
| GAGAACGCTCCAGCACTGAttttt | (SEQ ID NO:218) |
| TCCATGTCGGTCCTGATGCtttttttt | (SEQ ID NO:219) |
| TCCATGTCGGTCCTGCTGattttt | (SEQ ID NO:220) |
| ATGGACTCTCCAGCGTTCTCttttt | (SEQ ID NO:221) |
| ATGGAAGGTCCAACGTTCTCttttt | (SEQ ID NO:222) |
| TCCATGGCGGTCCTGATGCTttttt | (SEQ ID NO:223) |
| TCCATGACGGTCCTGATGCTttttt | (SEQ ID NO:224) |
| TCCATGTCGATCCTGATGCTttttt | (SEQ ID NO:225) |
| TCCATGTCGCTCCTGATGCTttttt | (SEQ ID NO:226) |
| TCCATGTCGTTCCTGATGCttttt | (SEQ ID NO:227) |
| TCCATAACGTTCCTGATGCttttt | (SEQ ID NO:228) |
| TCCATGACGTCCCTGATGCttttt | (SEQ ID NO:229) |
| atatatatGCTAGACGTTAGCGT | (SEQ ID NO:230) |
| atatatatCAACGTT | (SEQ ID NO:231) |
| atatatatCCATGACGTTCCTGATGCT | (SEQ ID NO:232) |
| atatatatCGACTCTCGAGCGTTCTC | (SEQ ID NO:233) |
| atatatatGCATGACGTTGAGCT | (SEQ ID NO:234) |
| atatatatCAGCGCT | (SEQ ID NO:235) |
| atatatatGAGAACGCTGGACCTTCCAT | (SEQ ID NO:236) |
| atatatatatGAGAACGCTCGACCTTCCAT | (SEQ ID NO:237) |
| atatatatGAGAACGCTCGACCTTCGAT | (SEQ ID NO:238) |
| atatatatGAGAACGCTCCAGCACTGAT | (SEQ ID NO:239) |
| atatatatTCCATGTCGGTCCTGATGCT | (SEQ ID NO:240) |
| atatatatTCCATGTCGGTCCTGCTGAT | (SEQ ID NO:241) |
| atatatatATGGACTCTCCAGCGTTCTC | (SEQ ID NO:242) |
| atatatatATGGAAGGTCCAACGTTCTC | (SEQ ID NO:243) |
| atatatatTCCATGGCGGTCCTGATGCT | (SEQ ID NO:244) |
| atatatatTCCATGACGGTCCTGATGCT | (SEQ ID NO:245) |
| atatatatTCCATGTCGATCCTGATGCT | (SEQ ID NO:246) |
| atatatatTCCATGTCGCTCCTGATGCT | (SEQ ID NO:247) |
| atatatatTCCATGTCGTTCCTGATGCT | (SEQ ID NO:248) |
| atatatatTCCATAACGTTCCTGATGCT | (SEQ ID NO:249) |
| atatatatCCATGACGTCCCTGATGCT | (SEQ ID NO:250) |
| aaaaaaaGCTAGACGTTAGCGTttttttt | (SEQ ID NO:251) |
| aaaaaaaTCAACGTTttttttt | (SEQ ID NO:252) |
| aaaaaaaTCCATGACGTTCCTGATGCTttttttt | (SEQ ID NO:253) |
| aaaaaaaATCGACTCTCGAGCGTTCTCttttttt | (SEQ ID NO:254) |
| aaaaaaaGCATGACGTTGAGCTttttttt | (SEQ ID NO:255) |
| aaaaaaaTCAGCGCTttttttt | (SEQ ID NO:256) |
| aaaaaaaGAGAACGCTGGACCTTCCATttttttt | (SEQ ID NO:257) |
| aaaaaaaGAGAACGCTCGACCTTCCATttttttt | (SEQ ID NO:258) |
| aaaaaaaGAGAACGCTCGACCTTCGATttttttt | (SEQ ID NO:259) |
| aaaaaaaGAGAACGCTCCAGCACTGATttttttt | (SEQ ID NO:260) |
| aaaaaaaTCCATGTCGGTCCTGATGCTttttttt | (SEQ ID NO:261) |
| aaaaaaaTCCATGTCGGTCCTGCTGATttttttt | (SEQ ID NO:262) |
| aaaaaaaATGGACTCTCCAGCGTTCTCttttttt | (SEQ ID NO:263) |
| aaaaaaaATGGAAGGTCCAACGTTCTGttttttt | (SEQ ID NO:264) |
| aaaaaaaTCCATGGCGGTCCTGATGCTttttttt | (SEQ ID NO:265) |
| aaaaaaaTCCATGACGGTCCTGATGCTttttttt | (SEQ ID NO:266) |
| aaaaaaaTCCATGTCGATCCTGATGCTttttttt | (SEQ ID NO:267) |
| aaaaaaaTCCATGTCGCTCCTGATGCTttttttt | (SEQ ID NO:268) |
| aaaaaaaTCCATGTCGTTCCTGATGCTttttttt | (SEQ ID NO:269) |
| aaaaaaaGCTAGACGTTAGCGttttttt | (SEQ ID NO:270) |
| aaaaaaaCAACGttttttt | (SEQ ID NO:271) |
| aaaaaaaTCCATGACGTTCCTGATGCttttttt | (SEQ ID NO:272) |
| aaaaaaaTCGACTCTCGAGCGTTCTCttttttt | (SEQ ID NO:273) |
| aaaaaaaGCATGACGTTGAGCttttttt | (SEQ ID NO:274) |
| aaaaaaaTCAGCGCttttttt | (SEQ ID NO:275) |
| aaaaaaaGAGAACGCTGGACCTTCCAttttttt | (SEQ ID NO:276) |
| aaaaaaaGAGAACGCTCGACCTTCCAttttttt | (SEQ ID NO:277) |
| aaaaaaaGAGAACGCTGGACCTTCGAttttttt | (SEQ ID NO:278) |
| aaaaaaaGAGAACGCTCCAGCACTGAttttttt | (SEQ ID NO:279) |
| aaaaaaaTCCATGTCGGTCCTGATGCttttttt | (SEQ ID NO:280) |
| aaaaaaaTCCATGTCGGTCCTGCTGAttttttt | (SEQ ID NO:281) |
| aaaaaaaTGGACTCTCCAGCGTTCTCttttttt | (SEQ ID NO:282) |
| aaaaaaaTGGAAGGTCCAACGTTCTCttttttt | (SEQ ID NO:283) |
| aaaaaaaTCCATGGCGGTCCTGATGCttttttt | (SEQ ID NO:284) |
| aaaaaaaTCCATGACGGTCCTGATGCttttttt | (SEQ ID NO:285) |
| aaaaaaaTCCATGTCGATCCTGATGCttttttt | (SEQ ID NO:286) |
| aaaaaaaTCCATGTCGCTCCTGATGCttttttt | (SEQ ID NO:287) |
| aaaaaaaTCCATGTCGTTCCTGATGCttttttt | (SEQ ID NO:288) |
| aGCTAGACGTTAGCGT | (SEQ ID NO:289) |
| aTCAACGTT | (SEQ ID NO:290) |
| aTCCATGACGTTCCTGATGCT | (SEQ ID NO:291) |
| aATCGACTCTCGAGCGTTCTC | (SEQ ID NO:292) |
| aGCATGACGTTGAGCT | (SEQ ID NO:293) |
| aTCAGCGCT | (SEQ ID NO:294) |
| aGAGAACGCTGGACCTTCCAT | (SEQ ID NO:295) |
| aGAGAACGCTCGACCTTCCAT | (SEQ ID NO:296) |
| aGAGAACGCTCGAGCTTCGAT | (SEQ ID NO:297) |
| aGAGAACGCTGCAGCACTGAT | (SEQ ID NO:298) |
| aTCCATGTCGGTCCTGATGCT | (SEQ ID NO:299) |
| aTCCATGTCGGTCCTGCTGAT | (SEQ ID NO:300) |
| aATGGACTCTCCAGCGTTCTC | (SEQ ID NO:301) |
| aATGGAAGGTOCAACGTTCTC | (SEQ ID NO:302) |
| aTCCATGGCGGTCCTGATGCT | (SEQ ID NO:303) |
| aTCCATGACGGTCCTGATGCT | (SEQ ID NO:304) |
| aTCCATGTCGATCCTGATGCT | (SEQ ID NO:305) |
| aTCCATGTCGCTCCTGATGCT | (SEQ ID NO:306) |
| aTCCATGTCGTTCCTGATGCT | (SEQ ID NO:307) |
| GCTAGACGTTAGCGTa | (SEQ ID NO:308) |
| TCAACGTTa | (SEQ ID NO:309) |
| TCCATGACGTTCCTGATGCTa | (SEQ ID NO:310) |
| ATCGACTCTCGAGCGTTCTCa | (SEQ ID NO:311) |
| GCATGACGTTGAGCTa | (SEQ ID NO:312) |
| TCAGOGOTa | (SEQ ID NO:313) |
| GAGAACGCTGGACCTTCCATa | (SEQ ID NO:314) |
| GAGAACGCTCGACCTTCCATa | (SEQ ID NO:315) |
| GAGAACGCTCGACCTTCGATa | (SEQ ID NO:316) |
| GAGAACGCTCCAGCACTGATa | (SEQ ID NO:317) |
| TCCATGTGGGTCCTGATGCTa | (SEQ ID NO:318) |
| TCCATGTCGGTCCTGCTGATa | (SEQ ID NO:319) |
| ATGGACTCTCCAGCGTTCTCa | (SEQ ID NO:320) |
| ATGGAAGGTCCAACGTTCTCa | (SEQ ID NO:321) |
| TCCATGGCGGTCCTGATGCTa | (SEQ ID NO:322) |
| TCCATGACGGTCCTGATGCTa | (SEQ ID NO:323) |
| TCCATGTCGATCCTGATGCTa | (SEQ ID NO:324) |
| TCCATGTCGCTCCTGATGCTa | (SEQ ID NO:325) |
| TCCATGTCGTTCCTGATGCTa | (SEQ ID NO:326) |
| aGCTAGACGTTAGCGTa | (SEQ ID NO:327) |
| aTCAACGTTa | (SEQ ID NO:328) |
| aTCCATGACGTTCCTGATGCTa | (SEQ ID NO:329) |
| aATCGACTCTCGAGCGTTCTCa | (SEQ ID NO:330) |
| aGCATGACGTTGAGCTa | (SEQ ID NO:331) |
| aTCAGCGCTa | (SEQ ID NO:332) |
| aGAGAACGCTGGACCTTCCATa | (SEQ ID NO:333) |
| aGAGAACGCTCGACCTTCCATa | (SEQ ID NO:334) |
| aGAGAACGCTCGACCTTCGATa | (SEQ ID NO:335) |
| aGAGAACGCTCCAGCACTGATa | (SEQ ID NO:336) |
| aTCCATGTCGGTCCTGATGCTa | (SEQ ID NO:337) |
| aTCCATGTCGGTCCTGCTGATa | (SEQ ID NO:338) |
| aATGGACTCTCCAGCGTTCTCa | (SEQ ID NO:339) |
| aATGGAAGGTCCAACGTTCTCa | (SEQ ID NO:340) |
| aTCCATGGCGGTCCTGATGCTa | (SEQ ID NO:341) |
| aTCCATGACGGTCCTGATGCTa | (SEQ ID NO:342) |
| aTCCATGTCGATCCTGATGCTa | (SEQ ID NO:343) |
| aTCCATGTCGCTCCTGATGCTa | (SEQ ID NO:344) |
| aTCCATGTCGTTCCTGATGGTa | (SEQ ID NO:345) |
| TCCATGACGTTCCTGATGCttttttttaaaaaaaa | (SEQ ID NO:346) |
| GCTAGACGTTAGCGttttttttaaaaaaaa | (SEQ ID NO:347) |
| TCAACGTTttttttaaaaaaaa | (SEQ ID NO:348) |
| TCCATGACGTTCCTGATGCTttttttttggaaaaaaaa | (SEQ ID NO:349) |
| ATCGACTCTCGAGCGTTCTCttttttttaaaaaaaa | (SEQ ID NO:350) |
| GCATGACGTTGAGCTttttttttaaaaaaaa | (SEQ ID NO:351) |
| TCAGCGCTttttttttaaaaaaaa | (SEQ ID NO:352) |
| GAGAACGCTGGACCTTCCATttttttttaaaaaaaa | (SEQ ID NO:353) |
| GAGAACGCTCGACCTTCCATttttttttaaaaaaaa | (SEQ ID NO:354) |
| GAGAACGCTCGACCTTCGATttttttttaaaaaaaa | (SEQ ID NO:355) |
| GAGAACGCTCCAGCACTGATttttttttaaaaaaaaa | (SEQ ID NO:356) |
| TCCATGTCGGTCCTGATGCTttttttttaaaaaaaaa | (SEQ ID NO:357) |
| TCCATGTCGGTCCTGCTGATttttttttaaaaaaaaa | (SEQ ID NO:358) |
| ATGGACTCTCCAGCGTTCTCttttttttaaaaaaaa | (SEQ ID NO:359) |
| ATGGAAGGTCCAAGGTTCTCttttttttaaaaaaaa | (SEQ ID NO:360) |
| TCCATGGCGGTCCTGATGCTttttttttaaaaaaaaa | (SEQ ID NO:361) |
| TCCATGACGGTCCTGATGCTttttttttaaaaaaaaa | (SEQ ID NO:362) |
| TCCATGTCGATCCTGATGCTttttttttaaaaaaaaa | (SEQ ID NO:363) |
| TGCATGTCGCTCCTGATGCTttttttttaaaaaaaaa | (SEQ ID NO:364) |
| TCCATGTCGTTCCTGATGCTttttttttaaaaaaaaa | (SEQ ID NO:365) |
| ccccccccGCTAGACGTTAGCGT | (SEQ ID NO:366) |
| ccccccccTCAACGTT | (SEQ ID NO:367) |
| ccccccccTCCATGACGTTCCTGATGCT | (SEQ ID NO :368) |
| ccccccccATCGACTCTCGAGCGTTCTC | (SEQ ID NO:369) |
| ccccccccGCATGACGTTGAGCT | (SEQ ID NO:370) |
| ccccccccTCAGCGCT | (SEQ ID NO:371) |
| ccccccccGAGAACGCTGGACCTTCCAT | (SEQ ID NO:372) |
| ccccccccGAGAACGCTCGACCTTCCAT | (SEQ ID NO:373) |
| ccccccccGAGAACGCTCGACCTTCGAT | (SEQ ID NO:374) |
| ccccccccGAGAACGCTCCAGCACTGAT | (SEQ ID NO:375) |
| ccccccccTCCATGTCGGTCCTGATGCT | (SEQ ID NO:376) |
| ccccccccTCCATGTCGGTCCTGCTGAT | (SEQ ID NO:377) |
| ccccccccATGGACTCTCCAGCGTTCTC | (SEQ ID NO:378) |
| ccccccccATGGAAGGTCCAACGTTCTC | (SEQ ID NO:379) |
| ccccccccTCCATGGCGGTCCTGATGCT | (SEQ ID NO:380) |
| ccccccccTCCATGACGGTCCTGATGCT | (SEQ ID NO:381) |
| ccccccccTCCATGTCGATCCTGATGCT | (SEQ ID NO:382) |
| ccccccccTCCATGTCGCTCCTGATGCT | (SEQ ID NO:383) |
| ccccccccTCCATGTCGTTCCTGATGCT | (SEQ ID NO:384) |
| GCTAGACGTTAGCGTgtgtgtgt | (SEQ ID NO:385) |
| TCAACGTTgtgtgtgt | (SEQ ID NO:386) |
| TCCATGACGTTCCTGATGCTgtgtgtgt | (SEQ ID NO:387) |
| ATCGACTCTCGAGCGTTCTCgtgtgtgt | (SEQ ID NO:388) |
| GCATGACGTTGAGCTgtgtgtgt | (SEQ ID NO:389) |
| TCAGCGCTgtgtgtgt | (SEQ ID NO:390) |
| GAGAACGGTGGACCTTCCATgtgtgtgt | (SEQ ID NO:391) |
| GAGAACGCTCGACCTTCCATgtgtgtgt | (SEQ ID NO:392) |
| GAGAACGCTCGACCTTCGATgtgtgtgt | (SEQ ID NO:393) |
| GAGAACGCTCCAGCACTGATgtgtgtgt | (SEQ ID NO:394) |
| TCCATGTCGGTCCTGATGCTgtgtgtgI | (SEQ ID NO:395) |
| TCCATGTCGGTCCTGCTGATgtgtgtgt | (SEQ ID NO:396) |
| ATGGACTCTCCAGCGTTCTCtgtgtgtgt | (SEQ ID NO:397) |
| ATGGAAGGTCCAACGTTCTCgtgtgtgt | (SEQ ID NO:398) |
| TCCATGGCGGTCCTGATGCTgtgtgtgt | (SEQ ID NO:399) |
| TCCATGACGGTCCTGATGCTgtgtgtgt | (SEQ ID NO:400) |
| TCCATGTCGATCCTGATGCTgtgtgtgt | (SEQ ID NO:401) |
| TCCATGTCGCTCCTGATGCTgtgtgtgt | (SEQ ID NO:402) |
| TCCATGTCGTTCCTGATGCTgtgtgtgt | (SEQ ID NO:403) |
| GCTAGACGTTAGCGt | (SEQ ID NO:404) |
| TCAACGtt | (SEQ ID NO:405) |
| TCCATGACGTTCCTGATGCt | (SEQ ID NO:406) |
| ATCGACTCTCGAGCGTTCTc | (SEQ ID NO:407) |
| GCATGACGTTGAGCt | (SEQ ID NO:408) |
| TCAGCGCt | (SEQ ID NO:409) |
| GAGAACGCTGGACCTTCCat | (SEQ ID NO:410) |
| GAGAACGCTCGACCTTCCat | (SEQ ID NO:411) |
| GAGAACGCTCGACCTTCGAt | (SEQ ID NO:412) |
| GAGAACGCTCCAGCACTGatat | (SEQ ID NO:413) |
| TCCATGTCGGTCCTGATGOt | (SEQ ID NO:414) |
| TCCATGTCGGTCCTGCTGAt | (SEQ ID NO:415) |
| ATGGACTCTCCAGCGTTCtc | (SEQ ID NO:416) |
| ATGGAAGGTCCAACGTtctc | (SEQ ID NO:417) |
| TCCATGGCGGTCCTGATGCt | (SEQ ID NO:418) |
| TCCATGACGGTCCTGATGct | (SEQ ID NO:419) |
| TCCATGTCGATCCTGATGct | (SEQ ID NO:420) |
| TCCATGTCGCTCCTGATGCt | (SEQ ID NO:421) |
| TCCATGTCGTTCCTGATGCt | (SEQ ID NO:422) |
| gCTAGACGTTAGCGt | (SEQ ID NO:423) |
| tCAACGTt | (SEQ ID NO:424) |
| tCCATGACGTTCCTGATGCt | (SEQ ID NO:425) |
| aTCGACTCTCGAGCGTTCTc | (SEQ ID NO:426) |
| gCATGACGTTGAGCt | (SEQ ID NO:427) |
| gCAGCGCt | (SEQ ID NO:428) |
| gAGAACGCTGGACCTTCCAt | (SEQ ID NO:429) |
| gAGAACGCTCGACCTTCCAt | (SEQ ID NO:430) |
| gAGAACGCTCGACCTTCGAt | (SEQ ID NO:431) |
| gAGAACGCTCCAGCACTGAt | (SEQ ID NO:432) |
| tCCATGTCGGTCCTGATGCt | (SEQ ID NO:433) |
| tCCATGTCGGTCCTGCTGAt | (SEQ ID NO:434) |
| aTGGACTCTCCAGCGTTCTc | (SEQ ID NO:435) |
| aTGGAAGGTCCAACGTTCTc | (SEQ ID NO:436) |
| tCCATGGCGGTCCTGATGCt | (SEQ ID NO:437) |
| tCCATGACGGTCCTGATGCt | (SEQ iD NO:438) |
| tCCATGTCGATCCTGATGCt | (SEQ ID NO:439) |
| tCCATGTCGCTCCTGATGCt | (SEQ ID NO:440) |
| tCCATGTCGTTCCTGATGCt | (SEQ ID NO:441) |
| GCTAGACGTTAGCGTgctagacgttagcgt | (SEQ ID NO:442) |
| TCAACGTT tccatgacgttcctgatgct | (SEQ ID NO:443) |
| TCCATGACGTTCCTGATGCTtccatgacgttcctgatgct | (SEQ ID NO:444) |
| ATCGACTCTCGAGCGTTCTCatcgactctcgagcgttctc | (SEQ ID NO:445) |
| GCATGACGTTGAGCTgcatgacgttgagct | (SEQ ID NO:446) |
| TCAGCGCTtcagcgct | (SEQ ID NO:447) |
| GAGAACGCTGGACCTTCCATgagaacgctcgaccttccat | (SEQ ID NO:448) |
| GAGAACGCTCGACCTTCCATgagaacgctcgaccttcgat | (SEQ ID NO:449) |
| GAGAACGCTCGACCTTCGATgagaacgctccagcactgat | (SEQ ID NO:450) |
| GAGAACGCTCCAGCACTGATtccatgtcggtcctgatgct | (SEQ ID NO:451) |
| TCCATGTCGGTCCTGATGGTtccatgtcggtcctgctgat | (SEQ ID NO:452) |
| TCCATGTCGGTCCTGCTGATatggactctccagcgttctc | (SEQ ID NO:453) |
| ATGGACTCTCCAGCGTTCTGatggaaggtccaacgttctc | (SEQ ID NO:454) |
| ATGGAAGGTCCAACGTTCTCtccatggcggtcctgatgct | (SEQ ID NO:455) |
| TCCATGGCGGTCCTGATGCTtccatgacggtcctgatgct | (SEQ ID NO:456) |
| TCCATGACGGTCCTGATGCTtccatgtcgatcctgatgct | (SEQ ID NO:457) |
| TCCATGTCGATCCTGATGCTtccatgtcgctcctgatgct | (SEQ ID NO:458) |
| TOCATGTCGCTCCTGATGCTtccatgtcgttcctgatgct | (SEQ ID NO:459) |
| TCCATGTCGTTCCTGATGCTtccatgacgtccctgatgct | (SEQ ID NO:460) |
| GCTAGACGTTAGCGTTTcgctaacgtctagc | (SEQ ID NO:461) |
| TCAACGTTaacgttga | (SEQ ID NO:462) |
| GGTGCATCGATGCAGGGGGGtcgagcgttctc | (SEQ ID NO:463) |
| TCCATGACGTTCCTGATGCTagcatcaggaacgtcatgga | (SEQ ID NO:464) |
| ATCGACTCTCGAGCGTTCTCgagaacgctcgagagtcgat | (SEQ ID NO:465) |
| GCATGACGTTGAGCTagctcaacgtcatgc | (SEQ ID NO:466) |
| TCAGCGCTagcgctga | (SEQ ID NO:467) |
| GAGAACGCTGGACCTTCCATatggaaggtccagcgttctc | (SEQ ID NO:468) |
| GAGAACGCTCGACCTTCCATatggaaggtcgagcgttctc | (SEQ ID NO:469) |
| GAGAACGCTCGACCTTCGATatcgaaggtcgagcgttcac | (SEQ ID NO:470) |
| GAGAACGCTCCAGCACTGATatcagtgctggagcgttcac | (SEQ ID NO:471) |
| TCCATGTCGGTCCTGATGCTaggtgcagcc | (SEQ ID NO:472) |
| TCCATGTCGGTCCTGCTGATcatgga | (SEQ ID NO:473) |
| ATGGACTCTCCAGCGTTCTCagagtccta | (SEQ ID NO:474) |
| ATGGAAGGTCCAACGTTCTCttggaccttccat | (SEQ ID NO:475) |
| TCCATGGCGGTCCTGATGCTaaaccgccatgga | (SEQ ID NO:476) |
| TCCATGACGGTCCTGATGCTtcaggaccgacat | (SEQ ID NO:477) |
| TCCATGTCGATCCTGATGCTatcgac | (SEQ ID NO:478) |
| TCCATGTCGCTCCTGATGCTcatgga | (SEQ ID NO:479) |
| TCCATGTCGTTCCTGATGCTGGAACGACATGGA | (SEQ ID NO:480) |
| CTCGAGctcgagCTCGAG | (SEQ ID NO:481) |
| ATCGAGatcgagATCGAG | (SEQ ID NO:482) |
| CTCGAGctcgagCTCGAG | (SEQ ID NO:483) |
| ATCGATatcgatATCGAT | (SEQ ID NO:484) |
| CTCGATctcgatCTCGAT | (SEQ ID NO:485) |
| atcgagCTCGAG | (SEQ ID NO:486) |
| atcgagATCGAG | (SEQ ID NO:487) |
| atcgagCTCGAG | (SEQ ID NO:488) |
| atcgatATCGAT | (SEQ ID NO:489) |
| ctcgatCTCGAT | (SEQ ID NO:490) |
| atcgagcTCGAGatcgag | (SEQ ID NO:491) |
| atcgagATCGAG atcgag | (SEQ ID NO:492) |
| atcgatCTCGAG atcgat | (SEQ ID NO:493) |
| ggtgcatcgatgcaGGGGGG | (SEQ ID NO:494) |
| ggtgcagcggtgcaGGGGGG | (SEQ ID NO:495) |
| ggtgcaccggtgcaGGGGGG | (SEQ ID NO:496) |
| ggtgtgtcgatgcaGGGGGG | (SEQ ID NO:497) |
| ggtgcatcgacgcaGGGGGG | (SEQ ID NO:498) |
| ggtgcaccgatgcaGGGGGG | (SEQ ID NO:499) |
| GGGGtgcatcgatgcaGGGGGG | (SEQ ID NO:500) |
| tgcatcgatgcaGGGGG | (SEQ ID NO:501) |
| aatgcatcgatgcaGGGGGG | (SEQ ID NO:502) |
| tgcatcgatgcaGGGGGG | (SEQ ID NO:503) |
| ggtgcaccggtgcaGGGGGG | (SEQ ID NO:504) |
| ggtgcatcgatgcaGGGGGG | (SEQ ID NO:505) |
| ggtgCAGCGGTGCAGGGGGG | (SEQ ID NO:506) |
| ggtgCACCGGTGCAGGGGGG | (SEQ ID NO:507) |
| ggtgTGTCGATGCAGGGGGG | (SEQ ID NO:508) |
| ggtgCATCGAGGCAGGGGGG | (SEQ ID NO:509) |
| ggtgCACCGATGCAGGGGGG | (SEQ ID NO:510) |
| tgcaTCGATGCAGGGGG | (SEQ ID NO:511) |
| aatgCATCGATGCAGGGGGG | (SEQ ID NO:512) |
| tgcaTCGATGCAGGGGGG | (SEQ ID NO:513) |
| ggtgCACCGGTGCAGGGGGG | (SEQ ID NO:514) |
| ggtgcatcgatgcaGGGGGGaaaaaaaa | (SEQ ID NO:515) |
| ggtgcagcggtgcaGGGGGGaaaaaaaa | (SEQ ID NO:516) |
| ggtgcaccggtgcaGGGGGGaaaaaaaa | (SEQ ID NO:517) |
| ggtgtgtcgatgcaGGGGGGaaaaaaaa | (SEQ ID NO:518) |
| ggtgcatcgacgcaGGGGGGaaaaaaaa | (SEQ ID NO:519) |
| ggtgcaccgatgcaGGGGGGaaaaaaaa | (SEQ ID NO:520) |
| GGGGtgcatcgatgcaGGGGGGaaaaaaaa | (SEQ ID NO:521) |
| tgcatcgatgcaGGGGGaaaaaaaa | (SEQ ID NO:522) |
| aatgcatcgatgcaGGGGGGaaaaaaaa | (SEQ ID NO:523) |
| tgcatcgatgcaGGGGGGaaaaaaaa | (SEQ ID NO:524) |
| ggtgcaccggtgcaGGGGGGaaaaaaaa | (SEQ ID NO:525) |
| ggggtgcatcgatgcaGGGGGGaaaaaaaa | (SEQ ID NO:526) |
| tcaacgttGGTGCATCGATGCAGGGGGG | (SEQ ID NO:527) |
| tcaacgttGGTGCAGCGGTGCAGGGGGG | (SEQ ID NO:528) |
| tcaacgttGGTGCACCGGTGCAGGGGGG | (SEQ ID NO:529) |
| tcaacgttGGTGTGTCGATGCAGGGGGG | (SEQ ID NO:530) |
| tcaacgttGGTGCATCGACGCAGGGGGG | (SEQ ID NO:531) |
| tcaacgttGGTGCACCGATGCAGGGGGG | (SEQ ID NO:532) |
| tcaacgttGGGTGCATCGATGCAGGGGGG | (SEQ ID NO:533) |
| tcaacgttTGCATCGATGCAGGGGG | (SEQ ID NO:534) |
| tcaacgttAATGCATGGATGCAGGGGGG | (SEQ ID NO:535) |
| tcaacgttTGCATCGATGCAGGGGGG | (SEQ ID NO:536) |
| tcaacgttGGTGCACCGGTGCAGGGGGG | (SEQ ID NO:537) |
| GGTGCatcgatGCAGGGGGG | (SEQ ID NO:538) |
| GGTGcagcggtcgCAGGGGGG | (SEQ ID NO:539) |
| GGTGCaccggtGCAGGGGGG | (SEQ ID NO:540) |
| GGTGTGTcgATGCAGGGGGG | (SEQ ID NO:541) |
| GGTGCatcgacGCAGGGGGG | (SEQ ID NO:542) |
| GGTGCaccgatGCAGGGGGG | (SEQ ID NO:543) |
| GGGGTGCatcgatGCAGGGGGG | (SEQ ID NO:544) |
| TGCATcgatgcaGGGGG | (SEQ ID NO:545) |
| AATGCATcgATGCAGGGGGG | (SEQ ID NO:546) |
| TGCATCGAatCAGGGGGG | (SEQ ID NO:547) |
| tatatatccccccGGTGCACCGGTGCAGGGGGGatatata | (SEQ ID NO:548) |
| tGCATCGATGCAGGGGG | (SEQ ID NO:549) |
| aatGCATCGATGCAGGGGGG | (SEQ ID NO:550) |
| tGCATCGATGCAGGGGGG | (SEQ ID NO:551) |
| atcgacTCTCGAGCGTtctc | (SEQ ID NO:552) |
| tcGAGCGTTctc | (SEQ ID NO:553) |
| tcgactCTCGAGCGttctc | (SEQ ID NO:554) |
| actCTCGAGCgttctc | (SEQ ID NO:555) |
| tctCGAGCGttctc | (SEQ ID NO:556) |
| ctcGAGCGTTct | (SEQ ID NO:557) |
| tcGAGGCttctc | (SEQ ID NO:558) |
| GCGAGGCttctc | (SEQ ID NO:559) |
| TCGATGCttctc | (SEQ ID NO:560) |
| tgcTTCGAGctc | (SEQ ID NO:561) |
| tcGTTTGTTctc | (SEQ ID NO:562) |
| TCGTATGtactc | (SEQ ID NO:563) |
| ttGTTCGTTctc | (SEQ ID NO:564) |
| ttGTTCGtactc | (SEQ ID NO:565) |
| atcgactCTCGAGCGTTCTC | (SEQ ID NO:566) |
| tcgaGCGTTCTC | (SEQ ID NO:567) |
| tcgactCTCGAGCGTTCTC | (SEQ ID NO:568) |
| actCTCGAGCGTTCTC | (SEQ ID NO:569) |
| tctCGAGCGTTCTC | (SEQ ID NO:570) |
| ctcgAGCGTTCT | (SEQ ID NO:571) |
| tcgAGGCTTCTC | (SEQ ID NO:572) |
| gcgaggCTTCTC | (SEQ ID NO:573) |
| tcgATGCTTGTC | (SEQ ID NO:574) |
| tgcTTCGAGCTC | (SEQ ID NO:575) |
| tcgtttGTTCTC | (SEQ ID NO:576) |
| tcgtatGTACTC | (SEQ ID NO:577) |
| ttgttCGTTCTC | (SEQ ID NO:578) |
| ttGTTCGTACTC | (SEQ ID NO:579) |
| atcgactctcgagcgttctcATCGACTCTCGAGCGTTCTC | (SEQ ID NO:580) |
| aaccaaccaaTCGAGCGTTCTC | (SEQ ID NO:581) |
| aaccaaccaaACTCTCGAGCGTTCTC | (SEQ ID NO:582) |
| aaccaaccaaTCTCGAGCGTTCTC | (SEQ ID NO:583) |
| aaccaaccaaCTCGAGCGTTCT | (SEQ ID NO:584) |
| aaccaaccaaTCGAGGCTTCTC | (SEQ ID NO:585) |
| aaccaaccaaGCGAGGCTTCTC | (SEQ ID NO:586) |
| aaccaaccaaTCGATGCTTCTC | (SEQ ID NO:587) |
| aaccaaccaaTGCTTCGAGCTC | (SEQ ID NO:588) |
| aaccaaccaaTCGTTTGTTCTC | (SEQ ID NO:589) |
| aaccaaccaaTCGTATGTACTC | (SEQ ID NO:590) |
| aaccaaccaaTTGTTCGTTCTC | (SEQ ID NO:591) |
| aaccaaccaaTTGTTCGTACTC | (SEQ ID NO:592) |
| aATCGACTCTCGAGCGTTCTC | (SEQ ID NO:593) |
| tCGAGCGTTCTC | (SEQ ID NO:594) |
| tCGACTCTCGAGCGTTCTC | (SEQ ID NO:595) |
| aCTCTCGAGCGTTCTC | (SEQ ID NO:596) |
| tCTCGAGCGTTCTC | (SEQ ID NO:597) |
| cTCGAGCGTTCT | (SEQ ID NO:598) |
| tCGAGGCTTCTC | (SEQ ID NO:599) |
| tCGATGCTTCTC | (SEQ ID NO:600) |
| tGCTTCGAGCTC | (SEQ ID NO:601) |
| tCGTTTGTTCTC | (SEQ ID NO:602) |
| tCGTATGTACTC | (SEQ ID NO:603) |
| tTGTTCGTTCTC | (SEQ ID NO:604) |
| tTGTTCGTACTC | (SEQ ID NO:605) |
| ATCGACTCTCGAGCGTTCTCtttttttttt | (SEQ ID NO:606) |
| TCGAGCGTTCTCtttttttttt | (SEQ ID NO:607) |
| TCGACTCTCGAGCGTTCTCtttttttttt | (SEQ ID NO:608) |
| ACTCTCGAGCGTTCTCtttttttttt | (SEQ ID NO:609) |
| TCTCGAGCGTTCTCtttttttttt | (SEQ ID NO:610) |
| CTCGAGCGTTCTtttttttttt | (SEQ ID NO:611) |
| TCGAGGCTTCTCtttttttttt | (SEQ ID NO:612) |
| GCGAGGCTTCTCtttttttttt | (SEQ ID NO:613) |
| TCGATGCTTCTCtttttttttt | (SEQ ID NO:614) |
| TGCTTCGAGCTCttttttttt | (SEQ ID NO:615) |
| TCGTTTGTTCTCtttttttttt | (SEQ ID NO:616) |
| TCGTATGTACTCtttttttttt | (SEQ ID NO:617) |
| TTGTTCGTTCTCtttttttttt | (SEQ ID NO:618) |
| TTGTTCGTACTCtttttttttt | (SEQ ID NO:619) |
| GCTAGACGTTAGCGTaacgtt | (SEQ ID NO:620) |

The specification is most thoroughly understood in light of the teachings of the references cited within the specification, all of which are hereby incorporated by reference in their entirety. The embodiments within the specification provide an illustration of embodiments of the invention and should not be construed to limit the scope of the invention. The skilled artisan recognizes that many other embodiments are encompassed by the claimed invention and that it is intended that the specification and examples be considered as exemplary only, with a true scope and spirit of the invention being indicated by the following claims.

We claim:

1. An immunostimulatory composition comprising:
at least one oligonucleotide comprising both an RNA region and a DNA region, wherein at least one terminus of the oligonucleotide comprises RNA.

2. The composition of claim 1, wherein the DNA region comprises at least one unmethylated CpG dinucleotide.

3. The composition of claim 2, wherein the DNA region comprises at least one CpG sequence.

4. The composition of claim 2, wherein both termini comprise at least 1 RNA nucleotide.

5. The composition of claim 3, wherein at least one terminus comprises poly A RNA.

6. The composition of claim 1, wherein a linkage between at least two nucleotides of the oligonucleotide comprises a modification of the phosphate backbone.

7. The composition of claim 6, wherein the modification is a phosphorathioate modification.

8. An immunostimulatory composition comprising at least a first oligonucleotide and a second oligonucleotide, wherein both the first and second oligonucleotides each contain at least one RNA region and at least one DNA region, wherein at least one terminus of each oligonucleotide comprises RNA.

9. The immunostimulatory composition of claim 8, wherein each oligonucleotide elicits a different immune stimulation profile.

10. An adjuvant comprising at least one oligonucleotide comprising both an RNA region and a DNA region, wherein at least one terminus of the oligonucleotide comprises RNA.

11. A vaccine comprising:
at least one oligonucleotide comprising both an RNA region and a DNA region, wherein at least one terminus of the oligonucleotide comprises RNA, and wherein said oligonucleotide is associated with a physiological carrier or delivery system.

12. A method of stimulating innate immunity comprising:
administering at least one oligonucleotide comprising both an RNA region and a DNA region, wherein at least one terminus of the oligonucleotide comprises RNA, and wherein said oligonucleotide is associated with a physiological carrier or delivery system.

13. A method of stimulating global immunity comprising:
administering at least one oligonucleotide comprising both an RNA region and a DNA region, wherein at least one terminus of the oligonucleotide comprises RNA, and wherein said oligonucleotide is associated with a physiological carrier or delivery system.

14. A vaccine comprising:
1) at least one oligonucleotide comprising both an RNA region and a DNA region, wherein at least one terminus of the oligonucleotide comprises RNA and,
2) at least one target antigen.

15. A method of stimulating a cellular immune response comprising:
administrating
1) at least one oligonucleotide comprising both an RNA region and a DNA region, wherein at least one terminus of the oligonucleotide comprises RNA and,
2) at least one target antigen.

16. A method of stimulating a humoral immune response comprising:
administrating
1) at least one oligonucleotide comprising both an RNA region and a DNA region, wherein at least one terminus of the oligonucleotide comprises RNA and,
2) at least one target antigen.

17. A method of making a vaccine comprising:
associating
1) at least one oligonucleotide comprising both an RNA region and a DNA region, wherein at least one terminus of the oligonucleotide comprises RNA, and
2) a physiological carrier or delivery system.
